(19) United States
(12) Patent Application Publication
(10) Pub. No.: US 2024/0325404 A1
(43) Pub. Date: Oct. 3, 2024

(54) TREATMENT OF INFLAMMATORY DISEASES

(71) Applicant: Galapagos NV, Mechelen (BE)

(72) Inventors: Justine DAO, Lausanne (CH); René Alexandre GALIEN, Romainville (FR); Magdalena Yonkova PETKOVA, Mechelen (BE); Christian Alexander SEEMAYER, Basel (CH); Edouard HELLOT, Romainville (FR); Natalia RUEDA RINCON, Hoboken, Antwerp (BE)

(21) Appl. No.: 18/578,604

(22) PCT Filed: Jul. 11, 2022
(86) PCT No.: PCT/EP2022/069362

§ 371 Date: Jan. 11, 2024

(30) Foreign Application Priority Data
Jul. 12, 2021 (EP) 21305971.0
Feb. 24, 2022 (EP) 22315039.2

(51) Int. Cl. A61K 31/5377; A61K 31/496

(52) U.S. Cl. CPC A61K 31/5377; A61K 31/496

(57) ABSTRACT

The present invention relates to new and improved methods for the use of Compound 1 in the treatment of inflammatory diseases and/or diseases associated with hypersecretion of IFNα and/or interferons (“interferonopathies”, especially type I interferonopathies), IL-12 and/or IL-23, and pharmaceutical unit dosage compositions comprising compound 1 for use therein.

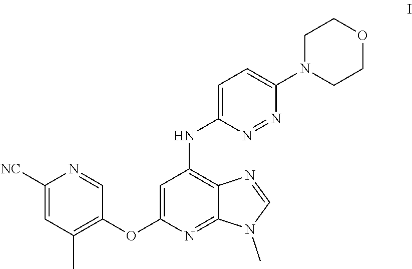 [SMILES: Cc1cc(C#N)ncc1Oc1cc(Nc2ccc(N3CCOCC3)nn2)c2ncn(C)c2n1]

### FIELD OF THE INVENTION

The present invention relates to novel and improved methods in the treatment of inflammatory diseases, and/or diseases associated with hypersecretion of IFNα and/or interferons (“interferonopathies”, especially type I interferonopathies), IL-12 and/or IL-23, and pharmaceutical unit dosage compositions comprising compound 1 for use therein.

### BACKGROUND OF THE INVENTION

Janus kinases (JAKs) are cytoplasmic tyrosine kinases that transduce cytokine signalling from membrane receptors to STAT transcription factors. Four JAK family members are described, JAK1, JAK2, JAK3 and TYK2. Upon binding of the cytokine to its receptor, JAK family members auto- and/or transphosphorylate each other, followed by phosphorylation of STATs that then migrate to the nucleus to modulate transcription. JAK-STAT intracellular signal transduction serves the interferons, most interleukins, as well as a variety of cytokines and endocrine factors such as EPO, TPO, GH, OSM, LIF, CNTF, GM-CSF and PRL. (Vainchenker et al., 2008)

The combination of genetic models and small molecule JAK inhibitor research revealed the therapeutic potential of JAK inhibitors (JAKinibs) (Babon et al., 2014). The last decade has seen the development of JAKinibs with various degrees of selectivity profiles versus the JAK family members. In particular, whereas targeting multiple JAK may not be detrimental (Broekman et al., 2011), developing selective JAKinibs would be very desirable to develop treatment course tailored to the needs of the patient despite the challenge it represents (Fabian et al., 2005). For example, whereas JAK2 inhibition has proven useful in the treatment of polycythemia and myelofibrosis, undesirable effect associated with JAK2 inhibition were observed (O'Shea and Plenge, 2012) thus rendering compounds with JAK2 inhibition components unsuitable for the treatment of non-JAK2 mediated diseases.

Using TYK2 knock out mice, it has been shown that IL-6, IL-10, IL-11, IL12, IL-13, IL-19, IL-20, IL-22, IL-23, IL-27, IL-28, IL-29, IL-31, IL-35 and/or type 1 interferons signaling are dependent on TYK2 (Schwartz et al., 2016). However, it has recently been shown that whereas JAK1 is a key driver in IFNα, IL6, IL10 and IL22 signaling, TYK2 is involved in type I interferons (including IFNα, INFO), IL23 and IL12 signaling (Gillooly et al., 2016; Sohn et al., 2013). Since the activity of IL12 and IL23 is particularly increased in patients with auto-immune diseases (O'Shea and Plenge, 2012) such as psoriasis and/or inflammatory bowel disorders, selective TYK2 inhibition may be particularly advantageous in the treatment of these diseases while avoiding JAK2 dependent erythropoietin (EPO) and thrombopoietin (TPO) signaling (Neubauer et al., 1998; Parganas et al., 1998).

Furthermore, TYK2 has been reported as a target for multiple autoimmune disorders, providing protection against inflammatory diseases as well as type 2 diabetes with a limited impact on the immune system. (Dendrou et al., 2016)

Compound 1 is a small molecule inhibitor of JAK, a family of tyrosine kinases, more particularly TYK2, and is currently under investigation as a drug for the treatment of inflammatory diseases and/or diseases associated with hypersecretion of IFNα and/or interferons (“interferonopathies”, especially type I interferonopathies), IL-12 and/or IL-23. The identification and synthesis of compound 1 have previously been described in WO 2019/076716.

There is an ongoing unmet medical need for the development of new and improved therapies in the treatment of inflammatory diseases and/or diseases associated with hypersecretion of IFNα and/or interferons (“interferonopathies”, especially type I interferonopathies), IL-12 and/or IL-23, in particular diseases such as systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis and/or Crohn's disease.

Cytochrome P450 (CYP) enzymes are essential for the metabolism of many medicines and endogenous compounds (Danielson 2002). The cytochrome P450 3A family is the most abundant subfamily of the CYP isoforms in the liver. There are at least three isoforms: 3A4, 3A5 and 3A7 in adults, of which 3A4 is considered the most important of all CYP enzymes in the liver (Ince et al. 2013).

CYP enzymes can be inhibited or induced by drugs, which may result in clinically significant drug-drug interactions that may cause unanticipated adverse reactions or therapeutic failures (Lynch and Price 2007). It is therefore crucial to understand which combinations of drugs should be contraindicated or their co-administration avoided.

Various cytochrome P450 3A4 (CYP3A4) inhibitors are known (https://www.fda.gov/drugs/drug-interactions-labeling/drug-development-and-drug-interactions-table-substrates-inhibitors-and-inducers) and Itraconazole, given its strong CYP3A inhibition, has emerged as probe to evaluate clinical drug-drug interaction (DDI) studies (Liu et al. 2016).

In a study by (Hu et al. 2020) it has been shown that 64% from the FDA-approved small molecule drugs (2005-2016) is metabolized by CYP3A4, so therefore it is important to mitigate the CYP3A4-mediated victim-drug-drug interactions risk, if warranted by the desired clinical profile of the drug.

Exposure of a drug may be influenced by the co-administration of a CYP3A4 inhibitor (Teo, Ho, and Chan 2015), leading to under or overdosing of said drug; therefore it is essential to ensure stable dose and exposure to avoid undesirable side effects or toxicity.

P-glycoprotein (P-gp), also referred to as “Multidrug Resistance Protein (MDR1)” and by its gene name “ABCB1”, is a member of a class of transport molecules called “ATP Binding Cassette” transporters or “ABC” transporters, which are located in the cell membrane of various tissues of the human body, such as intestines, kidney, liver and at the blood-brain barrier. P-glycoprotein plays an important role transporting drug substances outside the cell (efflux) influencing their elimination from the body. Along with the CYP enzymes, P-glycoprotein is an important mediator of drug-drug interactions. The pharmacokinetics of a drug may be altered when co-administered with compounds which inhibit or induce P-glycoprotein (König, Miller, and Fromm 2013). It is therefore crucial to understand which combinations of drugs should be contraindicated or their co-administration avoided.

The FDA has recognized and listed various drugs as potential P-gp inhibitors (https://www.fda.gov/drugs/drug-interactions-labeling/drug-development-and-drug-interactions-table-substrates-inhibitors-and-inducers).

In addition, the co-administration of a substance acting as P-gp inhibitor, which can also act as CYP inhibitor, may result in a reduced therapeutic effect due to a doble effect. On one side, a P-gp inhibition-mediated, enhanced the intracellular accumulation of the parent drug; and on another side, a CYP inhibition-mediated, may cause excessive drug accumulation of the parent drug and increased its toxicity, resulting in the need to reduce the dose of the therapeutic agent. Therefore, it is crucial to understand which combinations of drugs should be contraindicated or their co-administration avoided (Wandel et al. 1999).

### SUMMARY OF THE INVENTION

The invention described herein is based upon the findings that the Compound of the Invention:
i. when administered at certain dosages provides unexpected advantages in the treatment of inflammatory diseases and/or diseases associated with hypersecretion of IFNα and/or interferons (“interferonopathies”, especially type I interferonopathies), IL-12 and/or IL-23; and
ii. is metabolized by CYP3A4 and/or transported by P-gp, which could result in undesired drug-drug interactions when administered in combination with one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors.

Accordingly, in a first aspect the present invention provides the Compound of the Invention for use in the treatment of an inflammatory disease and/or a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis and/or Crohn's disease, wherein compound 1 is administered at a total daily dosage of at least 80 mg per day to 200 mg per day.

In a second aspect the present invention provides the compound of the invention for use in treating a patient in need of the compound of the invention therapy, characterized in that the treating comprises avoiding, contraindicating or discontinuing concomitant use or co-administration of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors.

In a further aspect, the present invention also provides the use of the Compound of the Invention in the manufacture of a medicament for the treatment of an inflammatory disease and/or a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis and/or Crohn's disease, wherein compound 1 is administered at a total daily dosage of at least 80 mg per day to 200 mg per day.

In a another aspect, the present invention provides a method of treating an inflammatory disease and/or a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis and/or Crohn's disease, comprising administering to a patient the Compound of the Invention at a total daily dosage of at least 80 mg per day to 200 mg per day.

In another aspect the present invention provides a pharmaceutical unit dosage composition comprising 80 mg to 200 mg of the Compound of the Invention, wherein the unit dosage form is suitable for oral administration up to a maximum total dosage of 200 mg per day.

In a further aspect, the present invention also provides the use of the compound of the invention in the manufacture of a medicament for treating a patient in need of therapy using the compound of the invention, characterized in that the treating comprises avoiding, contraindicating or discontinuing concomitant use or co-administration of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors.

In another aspect, the present invention provides a method of administering treatment using the compound of the invention to a patient in need of therapy using the compound of the invention comprising administering the patient a therapeutically effective amount of the compound of the invention, and, avoiding, contraindicating or discontinuing concomitant use or co-administration of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors.

In specific embodiments the patient in need of therapy is a patient suffering from inflammatory diseases, and/or diseases associated with hypersecretion of IFNα and/or interferons (“interferonopathies”, especially type I interferonopathies), IL-12 and/or IL-23. In particular, diseases such as systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis and/or Crohn's disease.

In specific embodiments, the one or more compounds that may produce potentially serious side effects or toxicity or exhibit adverse drug interactions when co-administered with the compound of the invention are CYP inhibitors and/or P-gp inhibitors, more particularly CYP3A4 inhibitors and/or P-gp inhibitors, even more particularly strong CYP3A4 inhibitors and/or strong P-gp inhibitors.

In specific embodiments the disease to be treated or the treatment is for a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, ulcerative colitis and/or Crohn's disease.

Compound 1 means a compound according to formula I below:

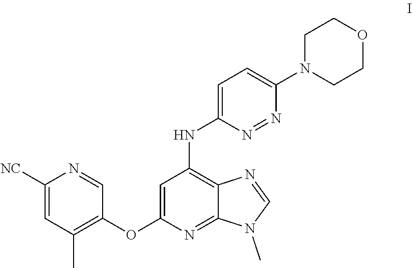 [SMILES: Cc1cc(C#N)ncc1Oc1cc(Nc2ccc(N3CCOCC3)nn2)c2ncn(C)c2n1]

The chemical name of Compound 1 is “4-methyl-5-[3-methyl-7-[(6-morpholin-4-ylpyridazin-3-yl)amino]imidazo[4,5-b]pyridin-5-yl]oxypyridine-2-carbonitrile”.

Moreover, Compound 1, useful in the pharmaceutical compositions and treatment methods disclosed herein, is pharmaceutically acceptable as prepared and used.

Other aspects, embodiments, objects and advantages will become apparent to those skilled in the art from a consideration of the ensuing detailed description.

### BRIEF DESCRIPTION OF THE DRAWINGS

FIG. 1. Shows the Mean (±SD) Compound 1 Plasma Concentration Versus Time Profiles—Part 1—(SAD)—PK Analysis Set (Linear-linear Scale)

FIG. 2. Shows the Mean (±SD) Compound 1 Plasma Concentration Versus Time Profiles—Part 1—(SAD)—PK Analysis Set (Log-linear Scale)

FIG. 3. Shows the Mean (±SD) Compound 1 Plasma Concentration Versus Time Profiles—Part 2 (MAD)—PK Analysis Set (Linear-linear Scale).

FIG. 4. Shows the Mean (±SD) Compound 1 Plasma Concentration Versus Time Profiles—Part 2 (MAD)—PK Analysis Set (Log-linear Scale).

FIG. 5. Shows Inhibition of the IFN-α-induced Neopterin Release Over Time Following Compound 1 Administration and IFN-α Challenge on Day 11—Part 2 (MAD)—PD Analysis Set.

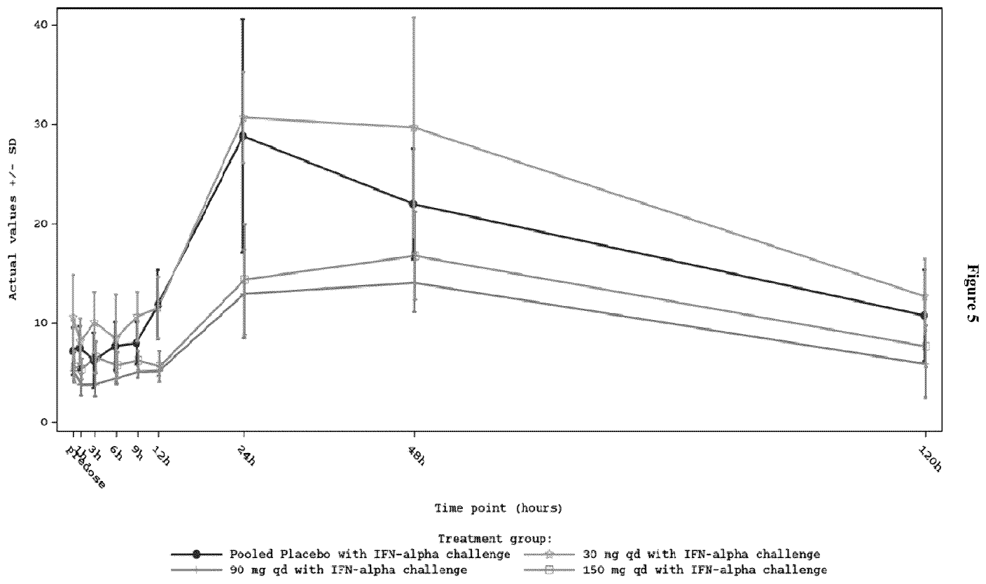

FIG. 6. Shows a two-dimensional heatmap displaying the effects of oral administration of Compound 1 on IFN-response genes in whole blood of volunteers after in vivo IFN-α challenge.

FIG. 7. Shows the PASI Cumulative Distribution of percentage change from baseline (% CfB) at Week 4 in the Phase 1b clinical study.

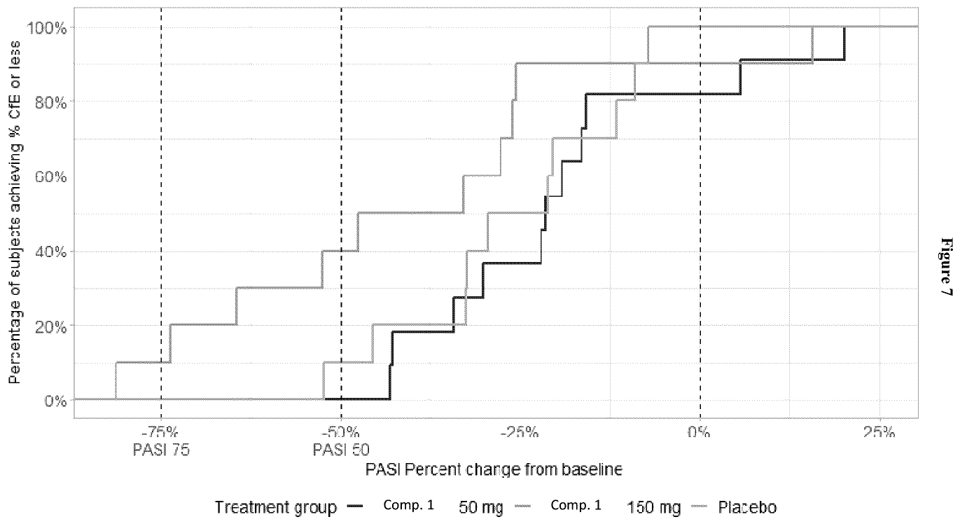

FIG. 8. Shows the PASI 50 Response Rate as % number of subjects with PASI 50 response at each visit.

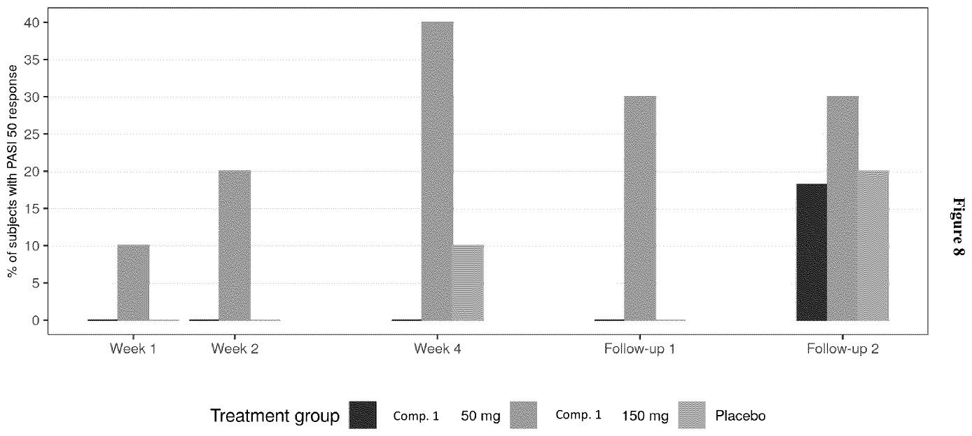

FIG. 9. Shows the PASI 75 Response Rate as % number of subjects with PASI 75 response at each visit.

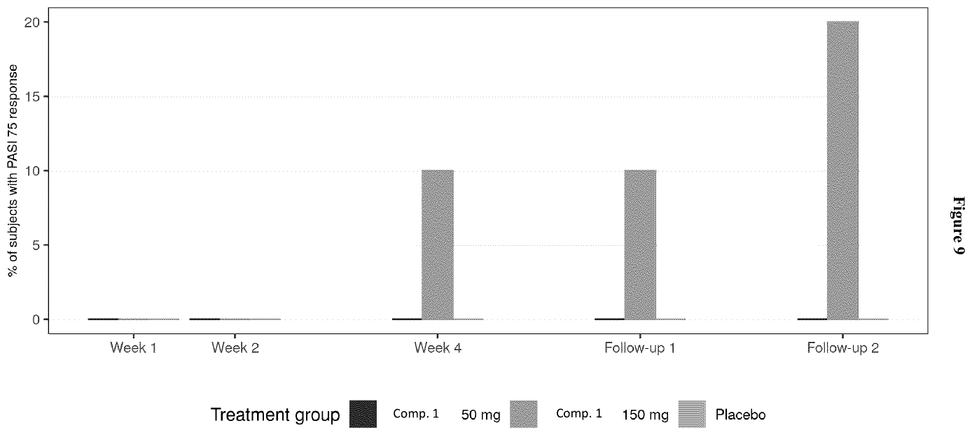

### DETAILED DESCRIPTION OF THE INVENTION

### Definitions

The following terms are intended to have the meanings presented therewith below and are useful in understanding the description and intended scope of the present invention.

When describing the invention, which may include the compound of formula I, pharmaceutical compositions containing said compound and methods of using said compound and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

The articles ‘a’ and ‘an’ may be used herein to refer to one or to more than one (i.e. at least one) of the grammatical objects of the article. By way of example ‘an analogue’ means one analogue or more than one analogue.

“The Compound of the Invention” means the compound of formula I or Compound 1, which expression includes the pharmaceutically acceptable salts/cocrystals, and the solvates, e.g. hydrates, and the solvates of the pharmaceutically acceptable salts/cocrystals, where the context so permits. Similarly, reference to intermediates, whether or not they themselves are claimed, is meant to embrace their salts/cocrystals, and solvates, where the context so permits.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Pharmaceutically acceptable salt/cocrystal’ refers to a salt and/or cocrystal of Compound 1 that is pharmaceutically acceptable and that possesses the desired pharmacological activity of the parent compound. In particular, such salts or cocrystals are non-toxic may be inorganic or organic acid addition salts and base addition salts.

‘Pharmaceutically acceptable vehicle’ refers to a diluent, adjuvant, excipient or carrier with which the compound of the invention is administered.

‘Solvate’ refers to forms of the compound that are associated with a solvent, usually by a solvolysis reaction. This physical association includes hydrogen bonding. Conventional solvents include water, EtOH, acetic acid and the like. The compound of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

“Cocrystal” refers to a crystalline material composed of Compound 1 and a co-crystal former (‘coformer’) in the same crystal lattice. The terms “cocrystal” and “co-crystal” are used interchangeably herein.

Reference to a certain ‘dose’ or ‘dosage’ of the Compound of the Invention refers to the equivalent weight of free base compound being administered, i.e. not including the weight of any salt, solvate or cocrystal counterpart or component.

‘Subject’ includes humans. The terms ‘human’, ‘patient’ and ‘subject’ are used interchangeably herein.

‘Effective amount’ means the amount of the Compound of the Invention that, when administered to a subject for treating a disease, is sufficient to effect such treatment for the disease. The “effective amount” can vary depending on the disease and its severity, and the age, weight, etc., of the subject to be treated.

‘Preventing’ or ‘prevention’ refers to a reduction in risk of acquiring or developing a disease or disorder (i.e. causing at least one of the clinical symptoms of the disease not to develop in a subject that may be exposed to a disease-causing agent, or predisposed to the disease in advance of disease onset.

The term ‘prophylaxis’ is related to ‘prevention’ and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Treating’ or ‘treatment’ of any disease or disorder refers, in one embodiment, to ameliorating the disease or disorder (i.e. arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g. stabilization of a discernible symptom), physiologically, (e.g. stabilization of a physical parameter), or both. In a further embodiment, “treating” or “treatment” relates to slowing the progression of the disease.

As used herein the term ‘inflammatory disease(s)’ refers to the group of conditions including, rheumatoid arthritis, osteoarthritis, juvenile idiopathic arthritis, psoriasis, psoriatic arthritis, ankylosing spondylitis, allergic airway disease (e.g. asthma, rhinitis), chronic obstructive pulmonary disease (COPD), inflammatory liver diseases (e.g. primary biliary cholangitis (PBC), and/or primary sclerosing cholangitis (PSC)), inflammatory bowel diseases (e.g. Crohn's disease, ulcerative colitis), endotoxin-driven disease states (e.g. complications after bypass surgery or chronic endotoxin states contributing to e.g. chronic cardiac failure), and related diseases involving cartilage, such as that of the joints. Particularly the term refers to rheumatoid arthritis, osteoarthritis, allergic airway disease (e.g. asthma), chronic obstructive pulmonary disease (COPD) and inflammatory bowel diseases. More particularly the term refers to rheumatoid arthritis, chronic obstructive pulmonary disease (COPD), primary biliary cholangitis (PBC), primary sclerosing cholangitis (PSC) and inflammatory bowel diseases. Most particularly the term refers to rheumatoid arthritis, chronic obstructive pulmonary disease (COPD) and inflammatory bowel diseases.

As used herein the term ‘disease(s) associated with hypersecretion of IFNα and/or interferons (“interferonopathies”, especially type I interferonopathies), IL-12 and/or IL-23’ includes conditions such as systemic and cutaneous lupus erythematosis, lupus nephritis, dermatomyositis, Sjogren's syndrome, psoriasis, rheumatoid arthritis, psoriatic arthritis, multiple sclerosis, trisomy 21, ulcerative colitis and/or Crohn's disease.

As used herein the term “Adverse Event (AE)” refers to any untoward medical occurrence in a patient or clinical investigation subject administered a pharmaceutical product and which does not necessarily have to have a causal relationship with this treatment. An Adverse Event (AE) can therefore be any unfavourable and/or unintended sign (including an abnormal laboratory finding, for example), symptom, or disease temporally associated with the use of a medicinal product, whether or not considered related to the medicinal product. AEs may also include pre- or post-treatment complications that occur as a result of protocol mandated procedures, lack of efficacy, Overdose or drug Abuse/Misuse reports. Pre-existing events that increase in severity or change in nature during or as a consequence of participation in the Clinical Study will also be considered AEs.

As used herein the term “Treatment Emergent Adverse Event (TEAE)” refers to any Adverse Event (AE) (or worsening of any Adverse Event (AE)) with an onset date on or after the start date of the respective treatment and no later than 30 days after the last dose of the respective treatment.

As used herein the term “Serious Adverse Event (SAEs)” refers to an Adverse Event (AE) that results in one of the following: death, a life-threatening event (an event in which the subject was at risk of death at the time of the event; it does not refer to an event that hypothetically might have caused death if it were more severe.), in-subject hospitalization or prolongation of existing hospitalization, persistent or significant disability/incapacity, a congenital anomaly/birth defect, or a medically significant event (medical and scientific judgment should be exercised in deciding whether other situations should be considered serious such as important medical events that might not be immediately life-threatening or result in death or hospitalization but might jeopardize the subject or might require intervention to prevent one of the other outcomes listed in the definition above).

As used herein, the term “avoid” and forms thereof are contemplated to have as alternatives the terms abstain, desist, forbear, and refrain, and forms thereof.

As used herein, the term “respective medicament” “said medicament” or “contraindicated medicament” refers to the medicament or the one or more compounds that may produce potentially serious side effects or toxicity or exhibit adverse drug interactions when co-administered with the compound of the invention.

As used herein, the term “avoiding the concomitant use or co-administration of” comprises or relates to avoidance of the use of the contraindicated medicament by looking into alternatives to the respective medicament in a patient in need of therapy with the respective medicament.

As used herein, the term “discontinue” and forms thereof, are contemplated to have as alternatives the terms cease, stop, suspend, and quit, and forms thereof.

As used herein, the term “contraindicating” and forms thereof such as “contraindication” are contemplated to contain the instruction to not enter in the contraindicated activity.

As used herein, the term “CYP inhibitors” refers to one or more compounds that increase the AUC of substrates of a given CYP. CYP inhibitors may be either weak, moderate or strong inhibitors. In particular, the term refers to CYP3A4 inhibitors. Examples of CYP3A4 inhibitors include Atazanavir, Boceprevir, Clarithromycin, Cobicistat, Conivaptan, Danoprevir, Darunavir, Delavirdine, Diltiazem, Elvitegravir, grapefruit juice, Idelalisib, Indinavir, Itraconazole, Ketoconazole, Lonafarnib, Lopinavir, Nefazodone, Nelfinavir, Nilotinib, Posaconazole, Ritonavir, Saquinavir, Stiripentol, Telithromycin, Tipranavir, Troleandomycin, Voriconazole, Aprepitant, Ciprofloxacin, Crizotinib, Cyclosporine, Dronedarone, Erythromycin, Fluconazole, Fluvoxamine, Imatinib, Verapamil, Chlorzoxazone, Cilostazol, Cimetidine, Fosaprepitant, Istradefylline, Ivacaftor, Lomitapide, Ranitidine, Ranolazine, Ticagrelor, (S)-omeprazole (esomeprazole)-high dose, ACT-178882, ACT-539313, Almorexant, AMD070, ANS-6637, Apararenone, ASP8477, Atorvastatin, AZD2327, Azithromycin, Berberine, Berotralstat, Bicalutamide, Brodalumab, Casopitant, Ceritinib, Clotrimazole, cranberry juice, Duvelisib, Entrectinib, Evacetrapid, Everolimus, Faldaprevir, Fedratinib, Fenebrutinib, FK1706, Fostamatinib, ginkgo (Ginkgo biloba), Glecaprevir/Pibrentasvir, Goldenseal (Hydrastis canadensis), Grazoprevir (ingredient of Zepatier), GSK2248761, Isavuconazole, Lapatinib, Larotrectinib, LCL161, Lefamulin, Letermovir, Lumateperone, Lurasidone, M100240, Mibefradil, Netupitant, obeticholic acid, Olaparib, Osilodrostat, Palbociclib, Pazopanib, Posaconazole, Propiverine, Ravuconazole, Ribociclib, Rimegepant, Roxithromycin, Rucaparib, Schisandra sphenanthera, Scutellarin (Breviscapine), Selpercatinib, Simeprevir, Suvorexant, Tabimorelin, Tacrolimus, Telaprevir, Teriflunomide, Tofisopam, Tucatinib, Verapamil and Voxelotor. More particular, the term refers to Atazanavir, Boceprevir, Clarithromycin, Cobicistat, Conivaptan, Danoprevir, Darunavir, Delavirdine, Diltiazem, Elvitegravir, grapefruit juice, Idelalisib, Indinavir, Itraconazole, Ketoconazole, Lonafarnib, Lopinavir, Nefazodone, Nelfinavir, Nilotinib, Posaconazole, Ribociclib, Ritonavir, Saquinavir, Stiripentol, Telaprevir, Telithromycin, Tipranavir, Troleandomycin, Voriconazole, Aprepitant, Ciprofloxacin, Crizotinib, Cyclosporine, Diltiazem, Dronedarone, Erythromycin, Fluconazole, Fluvoxamine, Imatinib, Tofisopam, Verapamil, Chlorzoxazone, Cilostazol, Cimetidine, Clotrimazole, Fosaprepitant, Istradefylline, Ivacaftor, Lomitapide, Ranitidine, Ranolazine, and Ticagrelor.

As used herein the term “weak CYP3A4 inhibitors” refers to one or more compounds that increase the AUC of oral Midazolam or other specific 3A4 substrate by 1.25 to 2-fold or results in a 20-50% reduction in its clearance. Examples of weak CYP3A4 inhibitors include Chlorzoxazone, Cilostazol, Clotrimazole, Cyclosporine, Fosaprepitant, Fluvoxamine, Istradefylline, Ivacaftor, Iomitapide, Ranitidine, Ranolazine, Ticagrelor (https://www.fda.gov/drugs/drug-interactions-labeling/drug-development-and-drug-interactions-table-substrates-inhibitors-and-inducers).

As used herein the term “moderate CYP3A4 inhibitors” refers to one or more compounds that increase the AUC of oral Midazolam or other specific 3A4 substrate by ≥2 to <5-fold or results in a 50-80% reduction in its clearance. Examples of moderate CYP3A4 inhibitors include Aprepitant, Cimetidine, Ciprofloxacin, Crizotinib, Diltiazem, Dronedarone, Erythromycin, Fluconazole, Imatinib, Tofisopam and Verapamil (https://www.fda.gov/drugs/drug-interactions-labeling/drug-development-and-drug-interactions-table-substrates-inhibitors-and-inducers).

As used herein the term “strong CYP3A4 inhibitors” refers to one or more compounds that increase the AUC of oral Midazolam or other specific 3A4 substrate by ≥5-fold or results in a ≥80% reduction in its clearance. Examples of strong CYP3A4 inhibitors include Boceprevir, Clarithromycin, Cobicistat, Conivaptan, Danoprevir, Elvitegravir, Idelalisib, Indinavir, Itraconazole, Ketoconazole, Lonafarnib, Lopinavir, Nefazodone, Nelfinavir, Posaconazole, Ribociclib, Ritonavir, Saquinavir, Telaprevir, Telithromycin, Tipranavir, Troleandomycin, Voriconazole, Ceritinib, grapefruit juice, LCL161, Mibefradil and Tucatinib (https://www.fda.gov/drugs/drug-interactions-labeling/drug-development-and-drug-interactions-table-substrates-inhibitors-and-inducers).

As used herein, the term “P-gp inhibitors” refers to one or more compounds that increase the AUC of substrates of P-gp, for example Midazolam and Prazosin. In addition, examples of P-pg inhibitors include Amiodarone, Azithromycin, Cannabidiol, Capmatinib, Carvedilol, Clarithromycin, Cobicistat, Cyclosporine, Daclatasvir, Diosmin, Dronedarone, Elagolix, Elagolix-Estradiol-Norethindrone, Eliglustat, Elexacaftor-tezacaftor-ivacaftor, Erythromycin, Flibanserin, Fostamatinib, Glecaprevir-pibrentasvir, Ketoconazole, Itraconazole, Ivacaftor, Ketoconazole, Lapatinib, Ledipasvir, Levoketoconazole, Neratinib, Ombitasvir-paritaprevir-ritonavir, Osimertinib, Propafenone, Quinidine, Quinine, Ranolazine, Ritonavir, Rolapitant, Roxithromycin, Simeprevir, Tamoxifen, Telithromycin, Tepotinib, Tezacaftor-Ivacaftor, Ticagrelor, Tucatinib, Velpatasvir, Vemurafenib, Verapamil, and Voclosporin (https://www.fda.gov/drugs/drug-interactions-labeling/drug-development-and-drug-interactions-table-substrates-inhibitors-and-inducers).

The term “strong P-gp inhibitor” refers to one or more compounds that increase the AUC of oral P-gp substrate by ≥5-fold, for example Midazolam and Prazosin. In addition, examples of strong P-gp inhibitors include Amiodarone, Azithromycin, Clarithromycin, Erythromycin, Roxithromycin, Telithromycin, Cyclosporine, Itraconazole, Ketoconazole, Tamoxifen, and Verapamil (https://www.fda.gov/drugs/drug-interactions-labeling/drug-development-and-drug-interactions-table-substrates-inhibitors-and-inducers).

A combined CYP3A4/P-gp inhibitor is a substance that upon administration to a subject decreases CYP3A4 and P-gp mediated activity. In a specific embodiment, the CYP3A4/P-gp inhibitor is Itraconazole.

The present disclosure includes all isotopic forms of the Compound of the Invention provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exists as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁸O), phosphorus-32 (³²P), sulphur-35 (³⁵S), chlorine-36 (³⁶Cl), chlorine-37 (³⁷Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in Positron Emission Topography (PET) studies for examining substrate receptor occupancy.

It is also to be understood that compounds that have the same molecular formula but differ in the nature or sequence of bonding of their atoms or the arrangement of their atoms in space are termed ‘isomers’. Isomers that differ in the arrangement of their atoms in space are termed ‘stereoisomers’.

‘Tautomers’ refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

Any ranges mentioned herein, above and below, include all values and sub-values between the lowest and highest limit of this range, including the limits.

### Compounds for Use in and Methods of Treatment

In one embodiment, the present invention provides the Compound of the Invention, or pharmaceutical compositions comprising the Compound of the Invention, for use in the treatment of an inflammatory disease and/or a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis and/or Crohn's disease, wherein compound 1 is administered at a total daily dosage of at least 80 mg per day to 200 mg per day.

In a particular embodiment, the disease is systemic lupus erythematosus, cutaneous lupus erythematosus, lupus nephritis, dermatomyositis, polymyositis, Sjogren's syndrome, psoriasis, rheumatoid arthritis, psoriatic arthritis, ulcerative colitis or Crohn's disease.

In a most particular embodiment, the disease is psoriatic arthritis. In another most particular embodiment, the disease is ulcerative colitis. In another most particular embodiment, the disease is psoriasis. In another most particular embodiment, the disease is Crohn's disease. In another most particular embodiment, the disease is systemic lupus erythematosus. In another most particular embodiment, the disease is cutaneous lupus erythematosus. In another most particular embodiment, the disease is lupus nephritis. In another most particular embodiment, the disease is dermatomyositis. In another most particular embodiment, the disease is polymyositis.

In another embodiment, the present invention provides the Compound of the Invention, or pharmaceutical compositions comprising the Compound of the Invention for use in the manufacture of a medicament for the treatment of an inflammatory disease and/or a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis and/or Crohn's disease, wherein compound 1 is administered at a total daily dosage of at least 80 mg per day to 200 mg per day. In a particular embodiment, the disease is systemic lupus erythematosus, cutaneous lupus erythematosus, lupus nephritis, dermatomyositis, polymyositis, Sjogren's syndrome, psoriasis, rheumatoid arthritis, psoriatic arthritis, ulcerative colitis or Crohn's disease. In a most particular embodiment, the disease is psoriatic arthritis. In another most particular embodiment, the disease is ulcerative colitis. In another most particular embodiment, the disease is psoriasis. In another most particular embodiment, the disease is Crohn's disease. In another most particular embodiment, the disease is systemic lupus erythematosus. In another most particular embodiment, the disease is cutaneous lupus erythematosus. In another most particular embodiment, the disease is lupus nephritis. In another most particular embodiment, the disease is dermatomyositis. In another most particular embodiment, the disease is polymyositis.

In additional method of treatment aspects, this invention provides methods of treating an inflammatory disease and/or a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis or Crohn's disease, comprising administering to a patient compound 1, or a pharmaceutically acceptable salt/cocrystal thereof, or a solvate or the solvate of a salt/cocrystal thereof, wherein compound 1 is administered at a total daily dosage of at least 80 mg per day to 200 mg per day. In a particular embodiment, the disease is systemic lupus erythematosus, cutaneous lupus erythematosus, lupus nephritis, dermatomyositis, polymyositis, Sjogren's syndrome, psoriasis, rheumatoid arthritis, psoriatic arthritis, ulcerative colitis or Crohn's disease. In a most particular embodiment, the disease is psoriatic arthritis. In another most particular embodiment, the disease is ulcerative colitis. In another most particular embodiment, the disease is psoriasis. In another most particular embodiment, the disease is Crohn's disease. In another most particular embodiment, the disease is systemic lupus erythematosus. In another most particular embodiment, the disease is cutaneous lupus erythematosus. In another most particular embodiment, the disease is lupus nephritis. In another most particular embodiment, the disease is dermatomyositis. In another most particular embodiment, the disease is polymyositis.

According to the methods of the present invention, the Compound of the Invention can be administered as the sole active agent or it can be administered in combination with other therapeutic agents, said other therapeutic agents may demonstrate the same or a similar therapeutic activity and that are determined to be safe and efficacious for such combined administration. In a specific embodiment, co-administration of two (or more) agents allows for lower doses of each to be used, thereby reducing any potential side effects from either medicament.

In one embodiment, the compound of the invention is administered orally. In a particular embodiment, the compound of the invention is administered orally to a patient in a fed state.

In one embodiment, the compound of the invention or a pharmaceutical composition comprising the compound of the invention is administered as a medicament. In a specific embodiment, said pharmaceutical composition additionally comprises a further active ingredient.

In one embodiment the Compound of the Invention is not an isotopic variant.

In one aspect the Compound of the Invention according to any one of the embodiments herein described is present as the free base.

In one aspect the Compound of the Invention according to any one of the embodiments herein described is a pharmaceutically acceptable salt or cocrystal.

In one aspect the Compound of the Invention according to any one of the embodiments herein described is a solvate of the compound.

In one aspect the Compound of the Invention according to any one of the embodiments herein described is a solvate of a pharmaceutically acceptable salt or cocrystal of the Compound of the Invention.

Alternatively, the exclusion of one or more of the specified variables from a group or an embodiment, or combinations thereof is also contemplated by the present invention.

### Pharmaceutical Compositions

When employed as a pharmaceutical, the Compound of the Invention is typically administered in the form of a pharmaceutical composition. Such compositions can be prepared in a manner well known in the pharmaceutical art and comprise at least one active compound of the invention according to Formula I. Generally, the Compound of the Invention is administered in a pharmaceutically effective amount. The amount of compound of the invention actually administered will typically be determined by a physician, in the light of the relevant circumstances, including the condition to be treated, the chosen route of administration, the actual compound of the invention administered, the age, weight, and response of the individual patient, the severity of the patient's symptoms, and the like.

The pharmaceutical compositions of this invention can be administered by a variety of routes including oral, rectal, transdermal, subcutaneous, intra-articular, intravenous, intramuscular, and intranasal. Depending on the intended route of delivery, the Compound of the Invention is preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term ‘unit dosage forms’ refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampules or syringes of the liquid compositions or pills, tablets, capsules or the like in the case of solid compositions. In such compositions, the compound of the invention according to Formula I is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or non-aqueous vehicle with buffers, suspending and dispensing agents, colorants, flavors and the like. Solid forms may include, for example, any of the following ingredients, or compound of the inventions of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint or orange flavoring.

The above-described components for orally administrable compositions are merely representative. Other materials as well as processing techniques and the like are set forth in Part 8 of Remington's Pharmaceutical Sciences, 17^{th} edition, 1985, Mack Publishing Company, Easton, Pennsylvania, which is incorporated herein by reference.

The Compound of the Invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

### Formulation 1—Tablets

The Compound of the Invention may be admixed as a dry powder with a dry gelatin binder in an approximate 1:2 weight ratio. A minor amount of magnesium stearate may be added as a lubricant. The mixture may be formed into 270 mg tablets (90 mg of active compound of the invention according to Formula I per tablet) in a tablet press.

### Formulation 2—Capsules

The Compound of the Invention may be admixed as a dry powder with a starch diluent in an approximate 1:1 weight ratio. The mixture may be filled into 250 mg capsules (125 mg of active compound of the invention according to Formula I per capsule).

### Formulation 3—Liquid

The Compound of the Invention (125 mg), may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color may be diluted with water and added with stirring. Sufficient water may then be added with stirring. Further sufficient water may be then added to produce a total volume of 5 mL.

### Formulation 4—Tablets

The Compound of the Invention may be admixed as a dry powder with a dry gelatin binder in an approximate 1:2 weight ratio. A minor amount of magnesium stearate may be added as a lubricant. The mixture may be formed into 450 mg tablets (150 mg of active compound of the invention according to Formula I) in a tablet press.

### Pharmaceutical Unit Dosage Compositions

The present invention further provides pharmaceutical unit dosage compositions.

In one embodiment, the present invention provides a pharmaceutical unit dosage composition comprising 80 mg to 200 mg of the Compound of the Invention.

In a particular embodiment, the unit dosage is in a form selected from a liquid, a tablet, a capsule, or a gelcap. In a most particular embodiment, the unit dosage is in the form of a tablet. In another most particular embodiment, the unit dosage is in the form of a capsule.

### Methods of Treatment

In one embodiment, the present invention provides the compound of the invention, or a pharmaceutical composition comprising the compound of the invention, for use in the treatment of a patient in need of therapy using the compound of the invention, characterized in that the treating comprises avoiding, contraindicating or discontinuing concomitant use or co-administration of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors. In a particular embodiment, the patient in need of therapy is suffering from one or more inflammatory diseases, and/or diseases associated with hypersecretion of INFα and/or interferons (“interferonopathies”, especially type I interferonopathies), IL-12 and/or IL-23. In a more particular embodiment, the disease is systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis or Crohn's disease.

In another embodiment, the present invention provides the compound of the invention, or a pharmaceutical composition comprising the compound of the invention for use in the manufacture of a medicament for use in the treatment of a patient in need of therapy using the compound of the invention, characterized in that the treatment comprises avoiding, contraindicating or discontinuing concomitant use or co-administration of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors. In a more particular embodiment, the patient in need of therapy is suffering from one or more inflammatory diseases, and/or diseases associated with hypersecretion of INFα and/or interferons (“interferonopathies”, especially type I interferonopathies), IL-12 and/or IL-23. In a more particular embodiment, the disease is systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis or Crohn's disease.

In additional method of treatment aspects, this invention provides methods of treatment of a patient in need of therapy thereof, which methods comprise the administration of an effective amount of the compound of the invention or one or more of the pharmaceutical compositions herein described wherein the treatment additionally comprises avoiding, contraindicating or discontinuing concomitant use or co-administration of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors. In a particular embodiment, the patient in need of therapy is suffering from one or more inflammatory diseases, and/or diseases associated with hypersecretion of INFα and/or interferons (“interferonopathies”, especially type I interferonopathies), IL-12 and/or IL-23. In more a particular embodiment, the disease is systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis or Crohn's disease.

In one embodiment, the invention provides a method of administering the compound of the invention to a patient in need of therapy thereof, comprising administering to the patient a therapeutically effective amount of the compound of the invention, and avoiding (concomitant) use or (co-)administration of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors.

In another embodiment, the invention provides a method of administering the compound of the invention to a patient in need of therapy thereof, comprising discontinuing administration of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, and then administering a therapeutically effective amount of the compound of the invention.

In one embodiment, the medicament or the one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, is discontinued concurrently with starting administration of the compound of the invention.

In another embodiment, the medicament or the one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, is discontinued at least 12 hours to 1 week prior to or after starting the compound of the invention therapy. This time period, for example, can permit adequate time for tapering and withdrawal without adverse effects.

In another embodiment, the medicament or the one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, is discontinued to avoid an adverse drug interaction or to avoid an adverse event; in particular, a treatment-emergent adverse event (TEAE), the contraindicated medicament is preferably discontinued within at least 3 days prior to starting the compound of the invention therapy.

In various embodiments, the medicament or the one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, is discontinued within at least 4 days, or at least 5 days, or at least 6 days, or at least 7 days (or one week), or at least 8 days, or at least 9 days, or at least 10 days, or at least 11 days, or at least 12 days, or at least 13 days, or at least 14 days (or two weeks), or at least 15 days, or at least 16 days, or at least 17 days, or at least 18 days, or at least 19 days, or at least 20 days, or at least 21 days (or three weeks), or at least 22 days, or at least 23 days, or at least 24 days, or at least 25 days, or at least 26 days, or at least 27 days, or at least 28 days (or four weeks), or at least 29 days, or at least 30 days, or at least one month, prior to starting the compound of the invention therapy.

In another various embodiments, the medicament or the one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, is discontinued within at least 2 half-lives, or at least 3 half-lives, or at least 4 half-lives, or at least 5 half-lives, or at least 6 half-lives, or at least 7 half-lives, or at least 8 half-lives, or at least 9 half-lives, or at least 10 half-lives, prior to starting the compound of the invention therapy.

In one embodiment, the medicament or the one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, is discontinued no earlier than one month, 3 weeks, 2 weeks or 1 week before starting the compound of the invention therapy. Preferably, sufficient time is allowed for tapering and/or withdrawal of the contraindicated medicament.

In embodiments where the medicament or the one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, cannot be or is not discontinued prior to the compound of the invention therapy, the contraindicated medicament is preferably discontinued within at least 3 days after starting Compound 1 therapy.

The patient preferably avoids the use of the medicament or the one or more of compounds that are CYP inhibitors and/or P-gp inhibitors, particularly CYP3A4 inhibitors and/or P-gp inhibitors, and more particularly strong CYP3A4 inhibitors and/or strong P-gp inhibitors to allow sufficient time to avoid adverse drug interactions or adverse events following starting the compound of the invention therapy.

In some aspects, the invention provides a method of administering the compound of the invention therapy to a patient in need of thereof and in need of therapy with another medicament that may produce serious side effects or toxicity or may exhibit adverse drug interactions when administered with or alongside CYP3A4 inhibitors and/or P-gp inhibitors, comprising administering a therapeutically effective amount of the compound of the invention to the patient, and administering an alternative therapy with a medicament of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors. In one embodiment according to these aspects, the method comprises concomitant use or co-administration of a medicament from the same class or mechanism of action or known to be a suitable alternative medicament for the respective therapy and that are not CYP inhibitors and/or not P-gp inhibitors, particularly not CYP3A4 inhibitors and/or not P-gp inhibitors, and more particularly not strong CYP3A4 inhibitors and/or not strong P-gp inhibitors. In another embodiment according to these aspects, the method comprises discontinuing treatment with the medicament that may produce serious side effects or toxicity or may exhibit adverse drug interactions when administered with or alongside CYP3A4 inhibitors and/or P-gp inhibitors and commencing treatment with a medicament from the same class or mechanism of action or known to be a suitable alternative medicament for the respective therapy and the medicament or the one or more compounds that are not CYP inhibitors and/or P-gp inhibitors, particularly not CYP3A4 inhibitors and/or not P-gp inhibitors, and more particularly not strong CYP3A4 inhibitors and/or not strong P-gp inhibitors.

The administration of a therapeutically effective amount of the compound of the invention to a patient in need of therapy thereof can be improved. In some embodiments, the patient is advised that co-administration of the compound of the invention with a medicament or the one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, can alter the therapeutic effect or adverse reaction profile of the compound of the invention and/or the respective medicament.

In some embodiments, the patient, if receiving therapy with a medicament or the one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, is advised that co-administration of the compound of the invention with the respective medicament can alter the therapeutic effect or adverse reaction profile of the compound of the invention and/or the respective medicament and that therapy with the respective medicament should be discontinued prior to commencing the compound of the invention therapy.

Injection dose levels range from about 0.01 mg/kg/h to at least 10 mg/kg/h, all for from about 1 to about 120 h and especially 24 to 96 h. A preloading bolus may also be administered to achieve adequate steady state levels. The maximum total dose is not expected to exceed about 1 g/day for a 40 to 80 kg human patient.

For the prophylaxis and/or treatment of long-term conditions, such as degenerative conditions, the regimen for treatment usually stretches over many months or years so oral dosing is preferred for patient convenience and tolerance. With oral dosing, one to four (1-4) regular doses daily, especially one to three (1-3) regular doses daily, typically one to two (1-2) regular doses daily, and most typically one (1) regular dose daily are representative regimens. Alternatively, for long lasting effect drugs, with oral dosing, once every other week, once weekly, and once a day are representative regimens. In particular, dosage regimen can be every 1-14 days, more particularly 1-10 days, even more particularly 1-7 days, and most particularly 1-3 days.

Using these dosing patterns, each dose provides from about 1 mg to about 1000 mg daily dose of the compound of the invention, with particular doses each providing from about 10 mg to about 600 mg daily dose. In a particular embodiment, the compound of the invention is administered from about 60 mg to 200 mg daily dose. In a more particular embodiment, the compound of the invention is administered at about 80 mg, 90 mg, 100 mg, 125 mg, 130 mg, 140 mg, 150 mg, 160 mg, 170 mg, 175 mg, 180 mg, 190 mg, or 200 mg daily dose for the treatment and/or prevention of inflammatory diseases, diseases associated with hypersecretion of IFNα and/or interferons (“interferonopathies”, especially type I interferonopathies), IL-12 and/or IL-23.

Transdermal doses are generally selected to provide similar or lower blood levels than are achieved using injection doses.

According to the methods of the present invention, the compound of the invention can be administered as the sole active agent or it can be administered in combination with other therapeutic agents, provided that concomitant use or co-administration treatment with a medicament or one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, is avoided, contraindicated or discontinued, said other therapeutic agents may demonstrate the same or a similar therapeutic activity and that are determined to be safe and efficacious for such combined administration. In a specific embodiment, co-administration of two (or more) agents allows for significantly lower doses of each to be used, thereby reducing the side effects seen.

In one embodiment, the compound of the invention or a pharmaceutical composition comprising the compound of the invention is administered as a medicament. In a specific embodiment, said pharmaceutical composition additionally comprises a further active ingredient, provided said further active ingredient is not selected from one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors.

In one embodiment the compound of the invention is not an isotopic variant.

In one aspect the compound of the invention according to any one of the embodiments herein described is present as the free base.

In one aspect the compound of the invention according to any one of the embodiments herein described is a pharmaceutically acceptable salt or cocrystal.

In one aspect the compound of the invention according to any one of the embodiments herein described is a solvate of the compound.

In one aspect the compound of the invention according to any one of the embodiments herein described is a solvate of a pharmaceutically acceptable salt or cocrystal of the compound of the invention.

Alternatively, the exclusion of one or more of the specified variables from a group or an embodiment, or combinations thereof is also contemplated by the present invention.

### Packages, Kits, Methods of Packaging, and Methods of Delivering

In another aspect, a package or kit is provided comprising the compound of the invention, optionally in a container, and, a package insert, package label, instructions or other labelling including information, recommendation or instruction regarding avoiding, contraindicating or discontinuing concomitant use or co-administration of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, in general, as described in different aspects and embodiments herein. Such as a package insert, package label, instructions or other labelling may include any one or more of the following information, recommendation, or instruction:
Informing or advising the patient that concurrent use of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, should be avoided;
Informing or advising the patient that concurrent use of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, is contraindicated;
Informing or advising the patient that concurrent use of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, should be discontinued, e.g. for at least 12 hours, 1 day, 2 days, 3 days, 4 days, 5 days, 6 days, 1 week, at least two weeks, at least 3 weeks or at least 4 weeks prior to compound 1 therapy;
Informing or advising the patient that concurrent use of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors, can alter the therapeutic effect of the respective medicament or of the compound of the invention, e.g. decreases the therapeutic effect of Compound 1 or the respective medicament, and/or leads to adverse drug interactions or adverse events, in which case the instruction may further state that therefore concurrent use is contraindicated;
Instructing the patient to discontinue concurrent use of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors;
Instructing the patient in need of Compound 1 therapy to not use or administer concurrently one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors;
Contra-indicating the concurrent use or administration of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors.

In the above-referenced embodiments and aspects of the invention the CYP3A4 inhibitor is selected from: Atazanavir, Boceprevir, Clarithromycin, Cobicistat, Conivaptan, Danoprevir, Darunavir, Delavirdine, Diltiazem, Elvitegravir, grapefruit juice, Idelalisib, Indinavir, Itraconazole, Ketoconazole, Lonafarnib, Lopinavir, Nefazodone, Nelfinavir, Nilotinib, Posaconazole, Ritonavir, Saquinavir, Stiripentol, Telithromycin, Tipranavir, Troleandomycin, Voriconazole, Aprepitant, Ciprofloxacin, Crizotinib, Cyclosporine, Dronedarone, Erythromycin, Fluconazole, Fluvoxamine, Imatinib, Verapamil, Chlorzoxazone, Cilostazol, Cimetidine, Fosaprepitant, Istradefylline, Ivacaftor, Lomitapide, Ranitidine, Ranolazine, Ticagrelor, (S)-omeprazole (esomeprazole)-high dose, ACT-178882, ACT-539313, Almorexant, AMD070, ANS-6637, Apararenone, ASP8477, Atorvastatin, AZD2327, Azithromycin, Berberine, Berotralstat, Bicalutamide, Brodalumab, Casopitant, Ceritinib, Clotrimazole, cranberry juice, Duvelisib, Entrectinib, Evacetrapid, Everolimus, Faldaprevir, Fedratinib, Fenebrutinib, FK1706, Fostamatinib, ginkgo (Ginkgo biloba), Glecaprevir/Pibrentasvir, Goldenseal (Hydrastis canadensis), Grazoprevir (ingredient of Zepatier), GSK2248761, Isavuconazole, Lapatinib, Larotrectinib, LCL161, Lefamulin, Letermovir, Lumateperone, Lurasidone, M100240, Mibefradil, Netupitant, obeticholic acid, Olaparib, Osilodrostat, Palbociclib, Pazopanib, Posaconazole, Propiverine, Ravuconazole, Ribociclib, Rimegepant, Roxithromycin, Rucaparib, Schisandra sphenanthera, Scutellarin (Breviscapine), Selpercatinib, Simeprevir, Suvorexant, Tabimorelin, Tacrolimus, Telaprevir, Teriflunomide, Tofisopam, Tucatinib, Verapamil and Voxelotor. In particular, the term refers to Atazanavir, Boceprevir, Clarithromycin, Cobicistat, Conivaptan, Danoprevir, Darunavir, Delavirdine, Diltiazem, Elvitegravir, grapefruit juice, Idelalisib, Indinavir, Itraconazole, Ketoconazole, Lonafarnib, Lopinavir, Nefazodone, Nelfinavir, Nilotinib, Posaconazole, Ribociclib, Ritonavir, Saquinavir, Stiripentol, Telaprevir, Telithromycin, Tipranavir, Troleandomycin, Voriconazole, Aprepitant, Ciprofloxacin, Crizotinib, Cyclosporine, Diltiazem, Dronedarone, Erythromycin, Fluconazole, Fluvoxamine, Imatinib, Tofisopam, Verapamil, Chlorzoxazone, Cilostazol, Cimetidine, Clotrimazole, Fosaprepitant, Istradefylline, Ivacaftor, Lomitapide, Ranitidine, Ranolazine, and Ticagrelor.

In a particular embodiment, the CYP3A4 inhibitor is selected from: Atazanavir, Boceprevir, Clarithromycin, Cobicistat, Conivaptan, Danoprevir, Darunavir, Delavirdine, Diltiazem, Elvitegravir, grapefruit juice, Idelalisib, Indinavir, Itraconazole, Ketoconazole, Lonafarib, Lopinavir, Nefazodone, Nelfinavir, Nilotinib, Posaconazole, Ribociclib, Ritonavir, Saquinavir, Stiripentol, Telaprevir, Telithromycin, Tipranavir, Troleandomycin, Voriconazole, Aprepitant, Ciprofloxacin, Crizotinib, Cyclosporine, Diltiazem, Dronedarone, Erythromycin, Fluconazole, Fluvoxamine, Imatinib, Tofisopam, Verapamil, Chlorzoxazone, Cilostazol, Cimetidine, Clotrimazole, Fosaprepitant, Istradefylline, Ivacaftor, Lomitapide, Ranitidine, Ranolazine, and Ticagrelor.

In a more particular embodiment, the strong CYP3A4 inhibitor is selected from: Boceprevir, Clarithromycin, Cobicistat, Conivaptan, Danoprevir, Elvitegravir, Idelalisib, Indinavir, Itraconazole, Ketoconazole, Lonafarib, Lopinavir, Nefazodone, Nelfinavir, Posaconazole, Ribociclib, Ritonavir, Saquinavir, Telaprevir, Telithromycin, Tipranavir, Troleandomycin, Voriconazole, Ceritinib, grapefruit juice, LCL161, Mibefradil and Tucatinib.

In the above-referenced embodiments and aspects of the invention the P-gp inhibitors is selected from: Amiodarone, Azithromycin, Cannabidiol, Capmatinib, Carvedilol, Clarithromycin, Cobicistat, Cyclosporine, Daclatasvir, Diosmin, Dronedarone, Elagolix, Elagolix-Estradiol-Norethindrone, Eliglustat, Elexacaftor-tezacaftor-ivacaftor, Erythromycin, Flibanserin, Fostamatinib, Glecaprevir-pibrentasvir, Ketoconazole, Itraconazole, Ivacaftor, Ketoconazole, Lapatinib, Ledipasvir, Levoketoconazole, Neratinib, Ombitasvir-paritaprevir-ritonavir, Osimertinib, Propafenone, Quinidine, Quinine, Ranolazine, Ritonavir, Rolapitant, Roxithromycin, Simeprevir, Tamoxifen, Telithromycin, Tepotinib, Tezacaftor-Ivacaftor, Ticagrelor, Tucatinib, Velpatasvir, Vemurafenib, Verapamil, and Voclosporin.

In a more particular embodiment, the strong P-gp inhibitor is selected from: Amiodarone, Azithromycin, Clarithromycin, Erythromycin, Roxithromycin, Telithromycin, Cyclosporine, Itraconazole, Ketoconazole, Tamoxifen, and Verapamil.

In the above-referenced embodiments and aspects of the invention the combined CYP3A4/P-gp inhibitor is a substance that upon administration to a subject decreases CYP3A4 and P-gp mediated activity.

In a particular embodiment, combined CYP3A4/P-gp inhibitor is itraconazole.

### Embodiments of the Invention According to the First Aspect

Further examples of embodiments of the invention include those given directly below:
001. Compound 1

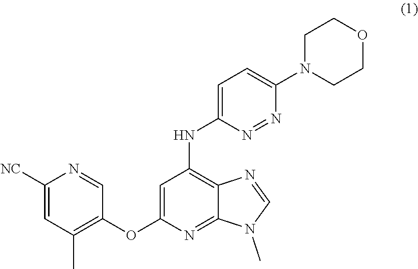 [SMILES: Cc1cc(C#N)ncc1Oc1cc(Nc2ccc(N3CCOCC3)nn2)c2ncn(C)c2n1]

or a pharmaceutically acceptable salt/cocrystal thereof, or a solvate or the solvate of a salt/cocrystal thereof, for use in the treatment of an inflammatory disease and/or a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis and/or Crohn's disease, wherein compound 1 is administered at a total daily dosage of at least 80 mg per day to 200 mg per day.
002. The use of compound 1, or a pharmaceutically acceptable salt/cocrystal thereof, or a solvate or the solvate of a salt/cocrystal thereof, in the manufacture of a medicament for the treatment of an inflammatory disease and/or a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis and/or Crohn's disease, wherein compound 1 is administered at a total daily dosage of at least 80 mg per day to 200 mg per day.
003. A method of treating an inflammatory disease and/or a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis and/or Crohn's disease, comprising administering to a patient compound 1, or a pharmaceutically acceptable salt/cocrystal thereof, or a solvate or the solvate of a salt/cocrystal thereof, wherein compound 1 is administered at a total daily dosage of at least 80 mg per day to 200 mg per day.
004. Compound 1 for use, use of compound 1 or method according to any of the preceding embodiments wherein the total daily dosage of compound 1 is administered as a once per day dosage (q.d.).
005. Compound 1 for use, use of compound 1 or method according to any of the preceding embodiments wherein compound 1 is administered at a total daily dosage of at least 90 mg per day to 200 mg per day, preferably 150 mg per day to 200 mg per day.
006. Compound 1 for use, use of compound 1 or method according to any of the preceding embodiments wherein compound 1 is administered at a total daily dosage of 90 mg per day, 100 mg per day, 110 mg per day, 120 mg per day, 125 mg per day, 130 mg per day, 140 mg per day, 150 mg per day, 160 mg per day, 170 mg per day, 175 mg per day, 180 mg per day, 190 mg per day or 200 mg per day.
007. Compound 1 for use, use of compound 1 or method according to any of the preceding embodiments wherein compound 1 is administered at a total daily dosage of 90 mg per day or 150 mg per day or 200 mg per day.
008. Compound 1 for use, use of compound 1 or method according to any of the preceding embodiments wherein compound 1 is administered at a total daily dosage of 150 mg or 200 mg per day.
009. Compound 1 for use, use of compound 1 or method according to any of the preceding embodiments wherein compound 1 is administered at a total daily dosage of 150 mg per day.
010. Compound 1 for use, use of compound 1 or method according to any of the preceding embodiments wherein the treatment is for psoriasis.
011. Compound 1 for use, use of compound 1 or method according to any of the preceding embodiments wherein the treatment is for psoriatic arthritis.
012. Compound 1 for use, use of compound 1 or method according to any of the preceding embodiments wherein the treatment is for Crohn's disease.
013. Compound 1 for use, use of compound 1 or method according to any of the preceding embodiments wherein the treatment is for ulcerative colitis.
014. Compound 1 for use, use of compound 1 or method according to any of the preceding embodiments wherein the treatment is for systemic lupus erythematosus.
015. Compound 1 for use, use of compound 1 or method according to any of the preceding embodiments wherein the treatment is for cutaneous lupus erythematosus.
016. Compound 1 for use, use of compound 1 or method according to any of the preceding embodiments wherein the treatment is for lupus nephritis.
017. Compound 1 for use, use of compound 1 or method according to any of the preceding embodiments wherein the treatment is for dermatomyositis.
018. Compound 1 for use, use of compound 1 or method according to any of the preceding embodiments wherein the treatment is for polymyositis.
019. Compound 1 for use, use of compound 1 or method according to any of the preceding embodiments wherein compound 1 is administered orally.
020. Compound 1 for use, use of compound 1 or method according to any of the preceding embodiments wherein compound 1 is administered orally to a patient in a fed state.
021. A pharmaceutical composition comprising a pharmaceutically acceptable carrier and a pharmaceutically effective amount of compound 1 for use, use of compound 1 or method according to any one of embodiments 001 to 020.
022. A pharmaceutical unit dosage composition comprising 80 mg to 200 mg of compound 1:

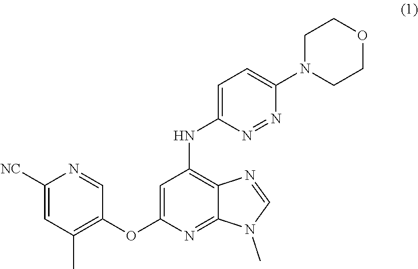 [SMILES: Cc1cc(C#N)ncc1Oc1cc(Nc2ccc(N3CCOCC3)nn2)c2ncn(C)c2n1]

or a pharmaceutically acceptable salt/cocrystal thereof, or a solvate or the solvate of a salt/cocrystal thereof, wherein the unit dosage form is suitable for oral administration up to a maximum total dosage of 200 mg of compound 1 per day.
023. The dosage form of embodiment 022 comprising from 90 mg to 200 mg of compound 1 in unit dosage form.
024. The dosage form of embodiment 022 or 023 comprising from 90 mg, 100 mg, 110 mg, 120 mg, 125 mg, 130 mg, 140 mg, 150 mg, 160 mg, 170 mg, 175 mg, 180 mg, 190 mg or 200 mg of compound 1 in unit dosage form.
025. The dosage form of embodiment 022 or 023 comprising 150 mg to 200 mg of compound 1 in unit dosage form.
026. The dosage form of embodiment 022, 023, 024 or 025 comprising 150 mg of compound 1 in unit dosage form.
027. The dosage form according to any one of embodiments 022, 023, 024, 025 or 026, wherein the unit dosage is in a form selected from a liquid, a tablet, a capsule, or a gelcap.
028. The dosage form according to any one of embodiments 022, 023, 024, 025, 026 or 027, wherein the unit dosage is in the form of a tablet or capsule.
029. The dosage form according to any one of embodiments 022, 023, 024, 025, 026, 027 or 028, wherein the unit dosage is in the form of a tablet.
030. The dosage form according to any one of embodiments 022, 023, 024, 025, 026, 027, 028 or 029 for use in the treatment of an inflammatory disease and/or a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis and/or Crohn's disease.
031. The use of the dosage form according to any one of 022, 023, 024, 025, 026, 027, 028 or 029, in the manufacture of a medicament for the treatment of an inflammatory disease and/or a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis and/or Crohn's disease.
032. A method of treating an inflammatory disease and/or a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, multiple sclerosis, trisomy 21, ulcerative colitis and/or Crohn's disease, comprising administering to a patient a dosage form according to any one of embodiments 022, 023, 024, 025, 026, 027, 028 or 029.

### Embodiments of the Invention According to the Second Aspect

Further examples of embodiments of the invention include those given directly below:
1. The compound of the invention according to Formula I

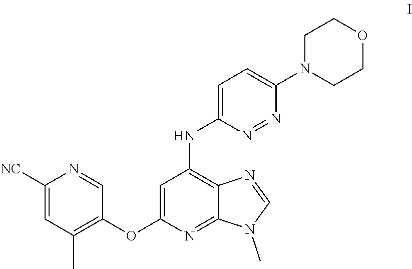 [SMILES: Cc1cc(C#N)ncc1Oc1cc(Nc2ccc(N3CCOCC3)nn2)c2ncn(C)c2n1]

or a pharmaceutically acceptable salt/cocrystal thereof, or a solvate or the solvate of a salt/cocrystal thereof, for use in treating a patient in need of therapy thereof, characterized in that the treating comprises avoiding or contraindicating or discontinuing concomitant use or co-administration of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors.
2. The use of the compound according to Formula I or a pharmaceutically acceptable salt/cocrystal thereof, or a solvate or the solvate of a salt/cocrystal thereof, in the manufacture of a medicament for treating a patient in need of therapy thereof, characterized in that the treating comprises avoiding, contraindicating or discontinuing concomitant use or co-administration of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors.
3. A method of administering treatment using the compound according to Formula I or a pharmaceutically acceptable salt/cocrystal thereof, or a solvate or the solvate of a salt/cocrystal thereof, to a patient in need of therapy thereof comprising administering the patient a therapeutically effective amount of compound 1, and avoiding, contraindicating or discontinuing concomitant use or co-administration of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors.
4. The compound for use according to embodiment 1, the use of the compound according to embodiment 2 or the method according to embodiment 3, wherein the patient in need of therapy thereof is currently undergoing treatment with one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors.
5. The compound for use according to embodiment 1 or 4, the use of the compound according to embodiment 2 or 4, or the method according to embodiment 3 or 4, wherein the use or method comprises the step of discontinuing the use of or treatment of one or more compounds that are CYP inhibitors and/or P-gp inhibitors, prior to or at the same time as the step of starting the therapy thereof, particularly CYP3A4 inhibitors and/or P-gp inhibitors, and more particularly strong CYP3A4 inhibitors and/or strong P-gp inhibitors.
6. The compound for use according to embodiment 1, 4 or 5, the use of the compound according to embodiment 2, 4 or 5, or the method according to embodiment 3, 4 or 5, wherein the use or method comprises the step of discontinuing the use of or treatment of one or more compounds that are CYP inhibitors and/or P-gp inhibitors, particularly CYP3A4 inhibitors and/or P-gp inhibitors, and more particularly strong CYP3A4 inhibitors and/or strong P-gp inhibitors; at least 12 hours, or at least 24 hours, prior to commencing the therapy thereof.
7. The compound for use according to embodiment 1, 4, 5 or 6, the use of the compound according to embodiment 2, 4, 5 or 6, or the method according to embodiment 3, 4, 5 or 6, wherein the use or method further comprises concomitant use or co-administration of a medicament from the same class or mechanism of action or known to be a suitable alternative medicament for the respective therapy thereof and that is a medicament or one or more compounds that are not CYP inhibitors and/or P-gp inhibitors, particularly not CYP3A4 inhibitors and/or not P-gp inhibitors, and more particularly not strong CYP3A4 inhibitors and/or strong not P-gp inhibitors.
8. The compound for use according to embodiment 1, 4, 5, 6 or 7, the use of the compound according to embodiment 2, 4, 5, 6 or 7, or the method according to embodiment 3, 4, 5, 6 or 7, wherein the use or method comprises discontinuing treatment with one or more compounds that are CYP inhibitors and/or P-gp inhibitors and commencing treatment with a medicament from the same class or mechanism of action or known to be a suitable alternative medicament for the respective therapy thereof and that is a medicament or one or more compounds that are not CYP inhibitors and/or P-gp inhibitors, particularly not CYP3A4 inhibitors and/or not P-gp inhibitors, and more particularly not strong CYP3A4 inhibitors and/or strong not P-gp inhibitors.
9. The compound for use according to embodiment 1, 4, 5, 6, 7 or 8, the use of the compound according to embodiment 2, 4, 5, 6, 7 or 8, or the method according to embodiment 3, 4, 5, 6, 7 or 8, wherein the patient in need of therapy thereof has one or more inflammatory diseases, diseases associated with hypersecretion of IFNα and/or interferons (“interferonopathies”, especially type I interferonopathies), IL-12 and/or IL-23.
10. The compound for use according to embodiment 1, 4, 5, 6, 7, 8 or 9, the use of the compound according to embodiment 2, 4, 5, 6, 7, 8 or 9, or the method according to embodiment 3, 4, 5, 6, 7, 8 or 9, wherein the patient in need of therapy thereof has systemic lupus erythematosus.
11. The compound for use according to embodiment 1, 4, 5, 6, 7, 8 or 9, the use of the compound according to embodiment 2, 4, 5, 6, 7, 8 or 9, or the method according to embodiment 3, 4, 5, 6, 7, 8 or 9, wherein the patient in need of therapy thereof has cutaneous lupus erythematosus.
12. The compound for use according to embodiment 1, 4, 5, 6, 7, 8 or 9, the use of the compound according to embodiment 2, 4, 5, 6, 7, 8 or 9, or the method according to embodiment 3, 4, 5, 6, 7, 8 or 9, wherein the patient in need of therapy thereof has lupus nephritis.
13. The compound for use according to embodiment 1, 4, 5, 6, 7, 8 or 9, the use of the compound according to embodiment 2, 4, 5, 6, 7, 8 or 9, or the method according to embodiment 3, 4, 5, 6, 7, 8 or 9, wherein the patient in need of therapy thereof has dermatomyositis.
14. The compound for use according to embodiment 1, 4, 5, 6, 7, 8 or 9, the use of the compound according to embodiment 2, 4, 5, 6, 7, 8 or 9, or the method according to embodiment 3, 4, 5, 6, 7, 8 or 9, wherein the patient in need of therapy thereof has polymyositis.
15. The compound for use according to embodiment 1, 4, 5, 6, 7, 8 or 9, the use of the compound according to embodiment 2, 4, 5, 6, 7, 8 or 9, or the method according to embodiment 3, 4, 5, 6, 7, 8 or 9, wherein the patient in need of therapy thereof has Sjogren's syndrome.
16. The compound for use according to embodiment 1, 4, 5, 6, 7, 8 or 9, the use of the compound according to embodiment 2, 4, 5, 6, 7, 8 or 9, or the method according to embodiment 3, 4, 5, 6, 7, 8 or 9, wherein the patient in need of therapy thereof has psoriasis.
17. The compound for use according to embodiment 1, 4, 5, 6, 7, 8 or 9, the use of the compound according to embodiment 2, 4, 5, 6, 7, 8 or 9, or the method according to embodiment 3, 4, 5, 6, 7, 8 or 9, wherein the patient in need of therapy thereof has rheumatoid arthritis.
18. The compound for use according to embodiment 1, 4, 5, 6, 7, 8 or 9, the use of the compound according to embodiment 2, 4, 5, 6, 7, 8 or 9, or the method according to embodiment 3, 4, 5, 6, 7, 8 or 9, wherein the patient in need of therapy thereof has psoriatic arthritis.
19. The compound for use according to embodiment 1, 4, 5, 6, 7, 8 or 9, the use of the compound according to embodiment 2, 4, 5, 6, 7, 8 or 9, or the method according to embodiment 3, 4, 5, 6, 7, 8 or 9, wherein the patient in need of therapy thereof has multiple sclerosis.
20. The compound for use according to embodiment 1, 4, 5, 6, 7, 8 or 9, the use of the compound according to embodiment 2, 4, 5, 6, 7, 8 or 9, or the method according to embodiment 3, 4, 5, 6, 7, 8 or 9, wherein the patient in need of therapy thereof has trisomy 21.
21. The compound for use according to embodiment 1, 4, 5, 6, 7, 8 or 9, the use of the compound according to embodiment 2, 4, 5, 6, 7, 8 or 9, or the method according to embodiment 3, 4, 5, 6, 7, 8 or 9, wherein the patient in need of therapy thereof has ulcerative colitis. 22. The compound for use according to embodiment 1, 4, 5, 6, 7, 8 or 9, the use of the compound according
to embodiment 2, 4, 5, 6, 7, 8 or 9, or the method according to embodiment 3, 4, 5, 6, 7, 8 or 9, wherein the patient in need of therapy thereof has Crohn's disease.
23. The compound for use according to embodiment 1, 4, 5, 6, 7, 8, 9, 10, 11, 12, 13, 14, 15, 16, 17, 18, 19, 20, 21 or 22, the use of the compound according to embodiment 2, 4, 5, 6, 7, 8, 9, 10, 11, 12, 13, 14, 15, 16, 17, 18, 19, 20, 21 or 22, or the method according to embodiment 3, 4, 5, 6, 7, 8, 9, 10, 11, 12, 13, 14, 15, 16, 17, 18, 19, 20, 21 or 22, wherein the cytochrome P450 (CYP) inhibitor is a CYP3A4 inhibitor and is one or more medicaments selected from: Atazanavir, Boceprevir, Clarithromycin, Cobicistat, Conivaptan, Danoprevir, Darunavir, Delavirdine, Diltiazem, Elvitegravir, grapefruit juice, Idelalisib, Indinavir, Itraconazole, Ketoconazole, Lonafarnib, Lopinavir, Nefazodone, Nelfinavir, Nilotinib, Posaconazole, Ritonavir, Saquinavir, Stiripentol, Telithromycin, Tipranavir, Troleandomycin, Voriconazole, Aprepitant, Ciprofloxacin, Crizotinib, Cyclosporine, Dronedarone, Erythromycin, Fluconazole, Fluvoxamine, Imatinib, Verapamil, Chlorzoxazone, Cilostazol, Cimetidine, Fosaprepitant, Istradefylline, Ivacaftor, Lomitapide, Ranitidine, Ranolazine, Ticagrelor, (S)-omeprazole (esomeprazole)-high dose, ACT-178882, ACT-539313, Almorexant, AMD070, ANS-6637, Apararenone, ASP8477, Atorvastatin, AZD2327, Azithromycin, Berberine, Berotralstat, Bicalutamide, Brodalumab, Casopitant, Ceritinib, Clotrimazole, cranberry juice, Duvelisib, Entrectinib, Evacetrapid, Everolimus, Faldaprevir, Fedratinib, Fenebrutinib, FK1706, Fostamatinib, ginkgo (Ginkgo biloba), Glecaprevir/Pibrentasvir, Goldenseal (Hydrastis canadensis), Grazoprevir (ingredient of Zepatier), GSK2248761, Isavuconazole, Lapatinib, Larotrectinib, LCL161, Lefamulin, Letermovir, Lumateperone, Lurasidone, M100240, Mibefradil, Netupitant, obeticholic acid, Olaparib, Osilodrostat, Palbociclib, Pazopanib, Posaconazole, Propiverine, Ravuconazole, Ribociclib, Rimegepant, Roxithromycin, Rucaparib, Schisandra sphenanthera, Scutellarin (Breviscapine), Selpercatinib, Simeprevir, Suvorexant, Tabimorelin, Tacrolimus, Telaprevir, Teriflunomide, Tofisopam, Tucatinib, Verapamil and Voxelotor.
24. The compound for use according to embodiment 1, 4, 5, 6, 7, 8, 9, 10, 11, 12, 13, 14, 15, 16, 17, 18, 19, 20, 21, 22 or 23, the use of the compound according to embodiment 2, 4, 5, 6, 7, 8, 9, 10, 11, 12, 13, 14, 15, 16, 17, 18, 19, 20, 21, 22 or 23, or the method according to embodiment 3, 4, 5, 6, 7, 8, 9, 10, 11, 12, 13, 14, 15, 16, 17, 18, 19, 20, 21, 22 or 23, wherein the P-gp inhibitor is one or more medicaments selected from: Amiodarone, Azithromycin, Cannabidiol, Capmatinib, Carvedilol, Clarithromycin, Cobicistat, Cyclosporine, Daclatasvir, Diosmin, Dronedarone, Elagolix, Elagolix-Estradiol-Norethindrone, Eliglustat, Elexacaftor-tezacaftor-ivacaftor, Erythromycin, Flibanserin, Fostamatinib, Glecaprevir-pibrentasvir, Ketoconazole, Itraconazole, Ivacaftor, Ketoconazole, Lapatinib, Ledipasvir, Levoketoconazole, Neratinib, Ombitasvir-paritaprevir-ritonavir, Osimertinib, Propafenone, Quinidine, Quinine, Ranolazine, Ritonavir, Rolapitant, Roxithromycin, Simeprevir, Tamoxifen, Telithromycin, Tepotinib, Tezacaftor-Ivacaftor, Ticagrelor, Tucatinib, Velpatasvir, Vemurafenib, Verapamil, and Voclosporin.
25. The compound for use according to embodiment 1, 4, 5, 6, 7, 8, 9, 10, 11, 12, 13, 14, 15, 16, 17, 18, 19, 20, 21, 22, 23 or 24, the use of the compound according to embodiment 2, 4, 5, 6, 7, 8, 9, 10, 11, 12, 13, 14, 15, 16, 17, 18, 19, 20, 21, 22, 23 or 24, or the method according to embodiment 3, 4, 5, 6, 7, 8, 9, 10, 11, 12, 13, 14, 15, 16, 17, 18, 19, 20, 21, 22, 23 or 24, wherein the compound according to Formula I or a pharmaceutically acceptable salt/cocrystal thereof, or a solvate or the solvate of a salt/cocrystal thereof is administered from about 80 mg to about 200 mg daily dose.
26. The compound for use according to embodiment 1, 4, 5, 6, 7, 8, 9, 10, 11, 12, 13, 14, 15, 16, 17, 18, 19, 20, 21, 22, 23, 24 or 25, the use of compound 1 according to embodiment 2, 4, 5, 6, 7, 8, 9, 10, 11, 12, 13, 14, 15, 16, 17, 18, 19, 20, 21, 22, 23, 24 or 25, or the method according to embodiment 3, 4, 5, 6, 7, 8, 9, 10, 11, 12, 13, 14, 15, 16, 17, 18, 19, 20, 21, 22, 23, 24 or 25, wherein the compound according to Formula I or a pharmaceutically acceptable salt/cocrystal thereof, or a solvate or the solvate of a salt/cocrystal thereof, is administered from about 80 mg, 90 mg, 100 mg, 125 mg, 130 mg, 140 mg, 150 mg, 160 mg, 170 mg, 175 mg, 180 mg, 190 mg, 200 mg daily dose.
27. A package or kit comprising:
   i. the compound according to Formula I or a pharmaceutically acceptable salt/cocrystal thereof, or a solvate or the solvate of a salt/cocrystal thereof, and
   ii. a package insert, package label, instructions or other labelling comprising instructions to avoid or discontinue or contraindication of concomitant use or co-administration of one or more compounds that are CYP inhibitors.
28. A package or kit according to embodiment 27 further comprising one or more of the features according to embodiments 1 to 26.
29. The compound for use according to embodiment 1, 4, 5, 6, 7, 8, 9, 10, 11, 12, 13, 14, 15, 16, 17, 18, 19, 20, 21, 22, 23, 24, 25, or 26, or the use of the compound according to embodiment 2, 4, 5, 6, 7, 8, 9, 10, 11, 12, 13, 14, 15, 16, 17, 18, 19, 20, 21, 22, 23, 24, 25, or 26, or the method according to embodiment 3, 4, 5, 6, 7, 8, 9, 10, 11, 12, 13, 14, 15, 16, 17, 18, 19, 20, 21, 22, 23, 24, 25, or 26, or a package or kit according to embodiment 27 or 28, wherein the CYP inhibitor is a CYP3A4 inhibitor.
30. The compound for use according to embodiment 1, 4, 5, 6, 7, 8, 9, 10, 11, 12, 13, 14, 15, 16, 17, 18, 19, 20, 21, 22, 23, 24, 25, 26, or 29, or the use of the compound according to embodiment 2, 4, 5, 6, 7, 8, 9, 10, 11, 12, 13, 14, 15, 16, 17, 18, 19, 20, 21, 22, 23, 24, 25, 26, or 29, or the method according to embodiment 3, 4, 5, 6, 7, 8, 9, 10, 11, 12, 13, 14, 15, 16, 17, 18, 19, 20, 21, 22, 23, 24, 25, 26, or 29, or a package or kit according to embodiment 27, 28 or 29, wherein the CYP inhibitor is a strong CYP3A4 inhibitor.
31. The compound for use according to embodiment 1, 4, 5, 6, 7, 8, 9, 10, 11, 12, 13, 14, 15, 16, 17, 18, 19, 20, 21, 22, 23, 24, 25, 26, 29 or 30, or the use of the compound according to embodiment 2, 4, 5, 6, 7, 8, 9, 10, 11, 12, 13, 14, 15, 16, 17, 18, 19, 20, 21, 22, 23, 24, 25, 26, 29 or 30, or the method according to embodiment 3, 4, 5, 6, 7, 8, 9, 10, 11, 12, 13, 14, 15, 16, 17, 18, 19, 20, 21, 22, 23, 24, 25, 26, 29 or 30, or a package or kit according to embodiment 27, 28, 29 or 30 wherein the cytochrome P450 (CYP) inhibitor is a CYP3A4 inhibitor and is one or more medicaments selected from Atazanavir, Boceprevir, Clarithromycin, Cobicistat, Conivaptan, Danoprevir, Darunavir, Delavirdine, Diltiazem, Elvitegravir, grapefruit juice, Idelalisib, Indinavir, Itraconazole, Ketoconazole, Lonafarnib, Lopinavir, Nefazodone, Nelfinavir, Nilotinib, Posaconazole, Ritonavir, Saquinavir, Stiripentol, Telithromycin, Tipranavir, Troleandomycin, Voriconazole, Aprepitant, Ciprofloxacin, Crizotinib, Cyclosporine, Dronedarone, Erythromycin, Fluconazole, Fluvoxamine, Imatinib, Verapamil, Chlorzoxazone, Cilostazol, Cimetidine, Fosaprepitant, Istradefylline, Ivacaftor, Lomitapide, Ranitidine, Ranolazine, Ticagrelor, (S)-omeprazole (esomeprazole)-high dose, ACT-178882, ACT-539313, Almorexant, AMD070, ANS-6637, Apararenone, ASP8477, Atorvastatin, AZD2327, Azithromycin, Berberine, Berotralstat, Bicalutamide, Brodalumab, Casopitant, Ceritinib, Clotrimazole, cranberry juice, Duvelisib, Entrectinib, Evacetrapid, Everolimus, Faldaprevir, Fedratinib, Fenebrutinib, FK1706, Fostamatinib, ginkgo (Ginkgo biloba), Glecaprevir/Pibrentasvir, Goldenseal (Hydrastis canadensis), Grazoprevir (ingredient of Zepatier), GSK2248761, Isavuconazole, Lapatinib, Larotrectinib, LCL161, Lefamulin, Letermovir, Lumateperone, Lurasidone, M100240, Mibefradil, Netupitant, obeticholic acid, Olaparib, Osilodrostat, Palbociclib, Pazopanib, Posaconazole, Propiverine, Ravuconazole, Ribociclib, Rimegepant, Roxithromycin, Rucaparib, Schisandra sphenanthera, Scutellarin (Breviscapine), Selpercatinib, Simeprevir, Suvorexant, Tabimorelin, Tacrolimus, Telaprevir, Teriflunomide, Tofisopam, Tucatinib, Verapamil and Voxelotor.
32. The compound for use according to embodiment 1, 4, 5, 6, 7, 8, 9, 10, 11, 12, 13, 14, 15, 16, 17, 18, 19, 20, 21, 22, 23, 24, 25, 26, 29, 30 or 31, or the use of the compound according to embodiment 2, 4, 5, 6, 7, 8, 9, 10, 11, 12, 13, 14, 15, 16, 17, 18, 19, 20, 21, 22, 23, 24, 25, 26, 29, 30 or 31, or the method according to embodiment 3, 4, 5, 6, 7, 8, 9, 10, 11, 12, 13, 14, 15, 16, 17, 18, 19, 20, 21, 22, 23, 24, 25, 26, 29, 30 or 31, or a package or kit according to embodiment 27, 28, 29, 30 or 31, wherein the cytochrome P450 inhibitor (CYP) is a CYP3A4 inhibitor and is one or more medicaments selected from Atazanavir, Boceprevir, Clarithromycin, Cobicistat, Conivaptan, Danoprevir, Darunavir, Delavirdine, Diltiazem, Elvitegravir, grapefruit juice, Idelalisib, Indinavir, Itraconazole, Ketoconazole, Lonafarnib, Lopinavir, Nefazodone, Nelfinavir, Nilotinib, Posaconazole, Ribociclib, Ritonavir, Saquinavir, Stiripentol, Telaprevir, Telithromycin, Tipranavir, Troleandomycin, Voriconazole, Aprepitant, Ciprofloxacin, Crizotinib, Cyclosporine, Diltiazem, Dronedarone, Erythromycin, Fluconazole, Fluvoxamine, Imatinib, Tofisopam, Verapamil, Chlorzoxazone, Cilostazol, Cimetidine, Clotrimazole, Fosaprepitant, Istradefylline, Ivacaftor, Lomitapide, Ranitidine, Ranolazine, and Ticagrelor.
33. The compound for use according to embodiment 1, 4, 5, 6, 7, 8, 9, 10, 11, 12, 13, 14, 15, 16, 17, 18, 19, 20, 21, 22, 23, 24, 25, 26, 29, 30, 31 or 32, or the use of the compound according to embodiment 2, 4, 5, 6, 7, 8, 9, 10, 11, 12, 13, 14, 15, 16, 17, 18, 19, 20, 21, 22, 23, 24, 25, 26, 29, 30, 31 or 32, or the method according to embodiment 3, 4, 5, 6, 7, 8, 9, 10, 11, 12, 13, 14, 15, 16, 17, 18, 19, 20, 21, 22, 23, 24, 25, 26, 29, 30, 31 or 32, or a package or kit according to embodiment 27, 28, 29, 30, 31 or 32, wherein the cytochrome P450 (CYP) inhibitor is a strong CYP3A4 inhibitor and is one or more medicaments selected from Boceprevir, Clarithromycin, Cobicistat, Conivaptan, Danoprevir, Elvitegravir, Idelalisib, Indinavir, Itraconazole, Ketoconazole, Lonafarnib, Lopinavir, Nefazodone, Nelfinavir, Posaconazole, Ribociclib, Ritonavir, Saquinavir, Telaprevir, Telithromycin, Tipranavir, Troleandomycin, Voriconazole, Ceritinib, grapefruit juice, LCL161, Mibefradil and Tucatinib.
34. The compound for use according to embodiment 1, 4, 5, 6, 7, 8, 9, 10, 11, 12, 13, 14, 15, 16, 17, 18, 19, 20, 21, 22, 23, 24, 25, 26, 29, 30, 31 32 or 33, or the use of the compound according to embodiment 2, 4, 5, 6, 7, 8, 9, 10, 11, 12, 13, 14, 15, 16, 17, 18, 19, 20, 21, 22, 23, 24, 25, 26, 29, 30, 3132 or 33, or the method according to embodiment 3, 4, 5, 6, 7, 8, 9, 10, 11, 12, 13, 14, 15, 16, 17, 18, 19, 20, 21, 22, 23, 24, 25, 26, 29, 30, 31 32 or 33, or a package or kit according to embodiment 27, 28, 29, 30, 31, 32 or 33, wherein the P-gp inhibitor is one or more medicaments selected from Amiodarone, Azithromycin, Cannabidiol, Capmatinib, Carvedilol, Clarithromycin, Cobicistat, Cyclosporine, Daclatasvir, Diosmin, Dronedarone, Elagolix, Elagolix-Estradiol-Norethindrone, Eliglustat, Elexacaftor-tezacaftor-ivacaftor, Erythromycin, Flibanserin, Fostamatinib, Glecaprevir-pibrentasvir, Ketoconazole, Itraconazole, Ivacaftor, Ketoconazole, Lapatinib, Ledipasvir, Levoketoconazole, Neratinib, Ombitasvir-paritaprevir-ritonavir, Osimertinib, Propafenone, Quinidine, Quinine, Ranolazine, Ritonavir, Rolapitant, Roxithromycin, Simeprevir, Tamoxifen, Telithromycin, Tepotinib, Tezacaftor-Ivacaftor, Ticagrelor, Tucatinib, Velpatasvir, Vemurafenib, Verapamil, and Voclosporin.
35. The compound for use according to embodiment 1, 4, 5, 6, 7, 8, 9, 10, 11, 12, 13, 14, 15, 16, 17, 18, 19, 20, 21, 22, 23, 24, 25, 26, 29, 30, 31 32, 33 or 34, or the use of the compound according to embodiment 2, 4, 5, 6, 7, 8, 9, 10, 11, 12, 13, 14, 15, 16, 17, 18, 19, 20, 21, 22, 23, 24, 25, 26, 29, 30, 31 32, 33 or 34, or the method according to embodiment 3, 4, 5, 6, 7, 8, 9, 10, 11, 12, 13, 14, 15, 16, 17, 18, 19, 20, 21, 22, 23, 24, 25, 26, 29, 30, 31 32, 33 or 34, or a package or kit according to embodiment 27, 28, 29, 30, 31, 32, 33 or 34, wherein the P-gp inhibitor is a strong P-gp inhibitor.
36. The compound for use according to embodiment 1, 4, 5, 6, 7, 8, 9, 10, 11, 12, 13, 14, 15, 16, 17, 18, 19, 20, 21, 22, 23, 24, 25, 26, 29, 30, 31 32, 33, 34 or 35, or the use of the compound according to embodiment 2, 4, 5, 6, 7, 8, 9, 10, 11, 12, 13, 14, 15, 16, 17, 18, 19, 20, 21, 22, 23, 24, 25, 26, 29, 30, 31 32, 33, 34 or 35, or the method according to embodiment 3, 4, 5, 6, 7, 8, 9, 10, 11, 12, 13, 14, 15, 16, 17, 18, 19, 20, 21, 22, 23, 24, 25, 26, 29, 30, 31 32, 33, 34 or 35, or a package or kit according to embodiment 27, 28, 29, 30, 31, 32, 33, 34 or 35, wherein the strong P-gp inhibitor and is one or more medicaments selected from Amiodarone, Azithromycin, Clarithromycin, Erythromycin, Roxithromycin, Telithromycin, Cyclosporine, Itraconazole, Ketoconazole, Tamoxifen, and Verapamil.
37. The compound for use according to embodiment 1, 4, 5, 6, 7, 8, 9, 10, 11, 12, 13, 14, 15, 16, 17, 18, 19, 20, 21, 22, 23, 24, 25, 26, 29, 30, 31 32, 33, 34, 35 or 36, or the use of the compound according to embodiment 2, 4, 5, 6, 7, 8, 9, 10, 11, 12, 13, 14, 15, 16, 17, 18, 19, 20, 21, 22, 23, 24, 25, 26, 29, 30, 31 32, 33, 34, 35 or 36, or the method according to embodiment 3, 4, 5, 6, 7, 8, 9, 10, 11, 12, 13, 14, 15, 16, 17, 18, 19, 20, 21, 22, 23, 24, 25, 26, 29, 30, 31 32, 33, 34, 35 or 36, or a package or kit according to embodiment 27, 28, 29, 30, 31, 32, 33, 34, 35 or 36, wherein the CYP3A4 inhibitor and/or P-gp inhibitor is a combined CYP3A4/P-gp inhibitor, in particular Itraconazole.

### Synthetic Preparation of the Compound of the Invention

The methods for the preparation of the Compound of the Invention have been described in WO 2019/076716 (the Compound of the Invention is referred to as “compound 38” in WO 2019/076716) and a summary is provided below.

### General Synthetic Methods

The compounds of the invention can be prepared from readily available starting materials using the following general methods and procedures. It will be appreciated that where typical or preferred process conditions (i.e. reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art (Greene, T W; Wuts, P G M;, 1991).

The following methods are presented with details as to the preparation of a compound of the invention as defined hereinabove and the comparative examples. A compound of the invention may be prepared from known or commercially available starting materials and reagents by one skilled in the art of organic synthesis.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Column chromatography is performed on silica gel 60 (35-70 μm). Thin layer chromatography is carried out using pre-coated silica gel F-254 plates (thickness 0.25 mm). ¹H NMR spectra were recorded on a a Bruker Advance 300 NMR spectrometer (300 MHz). Chemical shifts (δ) for ¹H NMR spectra are reported in parts per million (ppm) relative to tetramethylsilane (δ 0.00) or the appropriate residual solvent peak, i.e. CHCl₃ (δ 7.27), as internal reference. Multiplicities are given as singlet (s), doublet (d), triplet (t), quartet (q), quintuplet (quin), multiplet (in) and broad (br). Electrospray MS spectra were obtained on a Waters platform LC/MS spectrometer or with Waters Acquity H-Class UPLC coupled to a Waters Mass detector 3100 spectrometer. Columns used: Waters Acquity UPLC BEH C18 1.7 μm, 2.1 mm ID×50 mm L, Waters Acquity UPLC BEH C18 1.7 μm, 2.1 mm ID×30 mm L, or Waters Xterra MS 5 μm C18, 100×4.6 mm. The methods are using either MeCN/H₂O gradients (H₂O contains either 0.1% TFA or 0.1% NH₃) or MeOH/H₂O gradients (H₂O contains 0.05% TFA). Microwave heating is peformed with a Biotage Initiator.

Racemic mixtures were separated on a Agilent HP1100 system with UV detection. Column used: Chiralpak 1A (10×250 mm, 5 μm). Solvents used: iPrOH and tBME. Enantiomeric purity is determined on a Agilent HP1100 system with UV detection. Column used: Chiralpak IA (4.6×250 mm, 5 μm). Solvents used: iPrOH and tBME.

TABLE I
List of abbreviations used in the chemical
synthesis experimental section:

| Abbreviation | Definition |
|---|---|
| DCM | Dichloromethane |
| MeCN | Acetonitrile |
| DMF | N,N-dimethylformamide |
| TFA | Trifluoroacetic acid |
| THF | Tetrahydrofuran |
| NMR | Nuclear Magnetic Resonnance |
| DMSO | Dimethylsulfoxide |
| LC-MS | Liquid Chromatography- Mass Spectrometry |
| EtOAc | ethyl acetate |
| Rt | retention time |
| s | singlet |
| br s | broad singlet |
| d | duplet |
| dd | double duplet |
| m | multiplet |
| min | minute |
| mL | milliliter |
| μL | microliter |
| g | gram |
| mg | milligram |
| TEA | Triethylamine |
| mmol | millimoles |
| ppm | parts per million |
| Pd(OAc)₂ | Palladium(II) acetate |
| μm | micrometer |
| tBME | tert-Butylmethylether |
| iPrOH | iso-Propanol |
| TFA | Trifluoroacetic acid |
| NIS | N-Iodosuccinimide |
| XantPhos | 4,5-Bis(diphenylphosphino)-9,9-dimethylxanthene |
| TMHD | 2,2,6,6-Tetramethyl heptanedione |

### Synthesis of intermediates

### Intermediate 1: 7-Chloro-5-iodo-3-methyl-3H-imidazo[4,5-b]pyridine

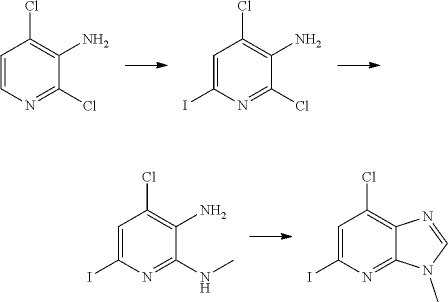

Step 1: 2,4-Dichloro-6-iodo-pyridin-3-ylamine: To a solution of 2,4-dichloro-3-aminopyridine (250 g, 1.54 mmol, 1 eq) in dry MeCN (1.2 L) under N₂ atmosphere at room temperature was added NIS (382 g, 1.70 mmol, 1.1 eq) and TFA (35.45 mL, 0.46 mmol, 0.3 eq). The mixture was stirred at 40° C. for 18 hours in 3 L round-bottom flask. Reaction mixture was then quenched with saturated Na₂S₂O₃ (500 mL) and NaHCO₃ (700 mL). Organic layer was washed with saturated NaHCO₃ and aqueous layers were washed twice with EtOAc (2×700 mL). Combined organic layers were dried over MgSO₄, filtered and concentrated to dryness to obtain crude product. It was purified by column chromatography using cyclohexane and EtOAc (10%) to give the desired product LCMS: m/z=289 [M+H].

### Step 2: 4-Chloro-6-iodo-N2-methyl-pyridine-2,3-diamine

2,4-dichloro-6-iodo-pyridin-3-amine (20 g, 0.07 mmol, 1 eq) was dissolved in n-butanol (300 mL) at autoclave (600 mL). Methylamine (33% in EtOH, 28.72 mL, 0.28 mmol, 4 eq) was added under N₂ ar room temperature. The mixture was stirred at 180° C. for 18 hours and then cooled to room temperature. This step was repeated twice and in the end, all the reaction mixtures were combined and concentrated to give 60 g of title compound that was used in next step as such. LCMS: m/z=284 [M+H].

Step 3: 7-Chloro-5-iodo-3-methyl-3H-imidazo[4,5-b]pyridine To a solution of 4-chloro-6-iodo-N-2-methyl-pyridine-2,3-diamine (60 g, 021 mmol, 1 eq) in formic acid (30 mL) was added trimethyl orthoformate (69.5 mL, 0.64 mmol, 3 eq). The mixture was stirred at 60° C. for 1 h. Reaction was concentrated to dryness after which the residue was diluted with DCM and quenched with saturated aquaeous NaHCO₃ solution. After extraction with DCM, organic layer was dried over Na₂SO₄, filtered and concentrated to dryness to afford crude material. It was purified by column chromatography using eluent cyclohexane/EtOAc from 10 to 60% of EtOAc to give the desired product. LCMS: m/z=294 [M+H]. ¹H NMR (300 MHz, DMSO-d₆) δ ppm: 8.46 (s, 1H), 7.83 (s, 1H), 3.81 (s, 3H).

### Intermediate 2: 5-(7-Chloro-3-methyl-3H-imidazo[4,5-b]pyridin-5-yloxy)-4-methyl-pyridine-2-carbonitrile

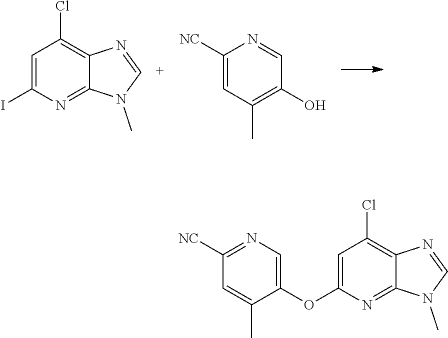

Intermediate 1 (68.51 g, 233.83 mmol, 1.0 eq), Intermediate 21 (47.00 g, 350.75 mmol, 1.5 eq), CuI (8.89 g, 46.77 mmol, 0.2 eq), TMHD (97.45 mL, 467.66 mmol, 2 eq) and Cs₂CO₃ (152 g, 467.66 mmol, 2 eq) were mixed together under air, DMF (234 mL) was added and the mixture was stirred at 85° C. for 2 nights. If full conversion was not reached, additional CuI (0.1 eq) and TMHD (1 eq) were added after which the mixture was stirred further at 85° C. for another night. Next, the mixture was cooled to 0° C. The resulting thick paste was then filtered and the cake was washed with ice cooled DMF (2×20 mL). It was then washed with ice cooled MTBE (3×150 mL). After drying the cake, it was suspended in 500 mL of 10% aqueous TMEDA solution. It was stirred for 2 h, filtered and the cake was washed with H₂O to afford the desired product. LCMS: m/z=300 [M+H]⁺.

### Intermediate 3: 5-(7-Amino-3-methyl-3H-imidazo[4,5-b]pyridin-5-yloxy)-4-methyl-pyridine-2-carbonitrile

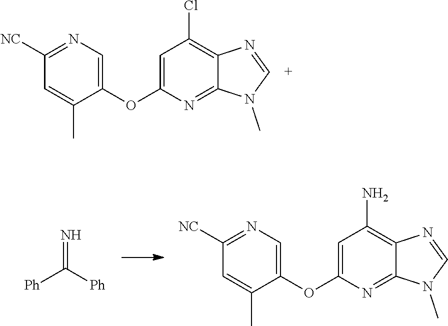 [SMILES: Cc1cc(C#N)ncc1Oc1cc(Cl)c2ncn(C)c2n1 ; N=C(c1ccccc1)c1ccccc1 ; Cc1cc(C#N)ncc1Oc1cc(N)c2ncn(C)c2n1]

To a mixture of intermediate 2 (5.0 g, 16.72 mmol, 1.0 eq), benzophenone imine (CAS [1013-88-3], 2.81 mL, 16.72 mmol, 1.0 eq), Pd₂CL₂(allyl)₂ (122 mg, 0.33 mmol, 0.02 eq), XantPhos (387 mg, 0.67 mmol, 0.04 eq) and Cs₂CO₃ (6.54 g, 20.07 mmol, 1.2 eq) under N₂ atmosphere, 1,4-dioxane (100 mL) was added and the mixture was stirred at 110° C. for 24 h. After letting it cool down till room temperature, the mixture was diluted with EtOAc and filtered over celite. The cake was washed with EtOAc (100 mL) and the filtrate was poured in 2N aquaeous HCl solution (200 mL), stirring it for 10 min. After extraction with EtOAc, the aquaeous phase was neutralized to pH=7 using NaHCO₃. This was followed by an extraction with EtOAc (5×100 mL) after which the combined organic layers were dried over MgSO₄, filtered and concentrated to dryness to afford the crude material which was triturated with DCM to afford the desired product. LCMS: m/z=281 [M+H]⁺.

### Alternative synthesis of Intermediate 3: 5-(7-Amino-3-methyl-3H-imidazo[4,5-b]pyridin-5-yloxy)-4-methyl-pyridine-2-carbonitrile

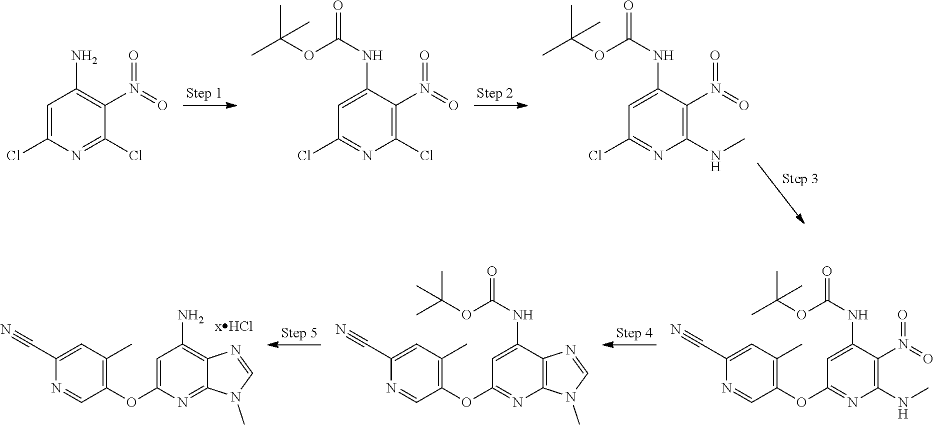 [SMILES: Nc1cc(Cl)nc(Cl)c1[N+](=O)[O-] ; CC(C)(C)OC(=O)Nc1cc(Cl)nc(Cl)c1[N+](=O)[O-] ; CNc1nc(Cl)cc(NC(=O)OC(C)(C)C)c1[N+](=O)[O-] ; Cc1cc(C#N)ncc1Oc1cc(N)c2ncn(C)c2n1 ; Cc1cc(C#N)ncc1Oc1cc(NC(=O)OC(C)(C)C)c2ncn(C)c2n1 ; CNc1nc(Oc2cnc(C#N)cc2C)cc(NC(=O)OC(C)(C)C)c1[N+](=O)[O-]]

Step 1: 2,6-Dichloro-4-amino-5-nitropyridine (520 g, 2.5 mol, 1.0 eq), was added to acetonitrile (5.2 L) at room temperature. To the mixture were added, under stirring at room temperature, BoC₂O (710 g, 3.25 mol, 1.3 eq) and K₃PO₄ (1000 g, 4.71 mol, 1.9 eq). The reaction mixture was heated at reflux for 1-2 hours. Then a solution of Boc₂O (110 g, 0.5 mol, 0.2 eq) in acetonitrile (100 mL) was added and the reaction mixture was heated at reflux for one additional hour. The reaction mixture was cooled down to room temperature and filtered on a pad Na₂SO₄. The Na₂SO₄ was washed with acetonitrile (2 L). The filtrate was evaporated under reduced pressure and redissolved in DCM (5 L). The DCM layer was washed with water. The organic layer was extracted with DCM (5 L) and the combined organic layers were dried over Na₂SO₄, filtered and evaporated to afford the desired product. LCMS: m/z=306/308 [M+H].

Step 2: 2,6-Dichloro-4 Boc-amino-5-nitropyridine (770 g, 2.5 mol, 1.0 eq) was added to isopropanol (11 L) at room temperature. To the mixture were added, under stirring at room temperature, methylamine 33% in EtOH (800 mL, 3.0 eq) over 1 h30. The reaction mixture was stirred at room temperature for 1 h30. The suspension was filtered, washed with isopropanol (1 L) then water (4 L). Following drying the desired product was obtained. LCMS: m/z=302.9/304.8 [M+H].

Step 3: tert-butyl N-[6-chloro-2-(methylamino)-3-nitro-4-pyridyl]carbamate (788 g, 2.6 mol, 1.0 eq), was added to acetonitrile (5.5 L) at room temperature. To the mixture were added, under stirring at room temperature, 5-hydroxy-4-methyl-pyridine-2-carbonitrile (384 g, 2.86 mol 1.1 eq) and Na₂CO₃ (414 g, 3.9 mol, 1.5 eq). The reaction mixture was heated at reflux for 48 hours. The reaction mixture was cooled down to room temperature and the insoluble were filtrered and washed with acetonitrile (2 L). The combined organic layers were evaporated. The crude was washed with water (5 L), collected and dried to afford the desired product. LCMS: m/z=401.1 [M+H]; m/z=399.2 [M−H]. ¹H NMR (400 MHz, DMSO-d₆) δ 10.70 (s, 1H), 9.05 (q, 1H), 8.62 (s, 1H), 8.13 (s, 1H), 7.23 (s, 1H), 2.59 (d, 3H), 2.24 (s, 3H), 1.50 (s, 9H).

Step 4: tert-butyl N-[6-[(6-cyano-4-methyl-3-pyridyl)oxy]-2-(methylamino)-3-nitro-4-pyridyl]carbamate (150 g, 375 mmol, 1.0 eq) was added to a mixture of acetic acid (750 mL, 35eg) and trimethyl orthoformate (750 mL, 18 eq) at room temperature. To the mixture were added by portions, under vigourous stirring at 20-21° C., Zn dust <10 μm (total of 120 g, 4.9eg, added by portions of 15 g). Each addition was performed after the reaction mixture had cooled down to 20-21° C. The reaction mixture was stirred during one hour after the last addition. The suspension was filtered on Dicalite 4158 (Carlo Erba, ref P8880014), washed with THF (1 L) and the combined organic lavers were evapored. The residue was slowly poured into a cold mixture of 20% ammoniac solution (100 mL) and water (2 L). The resulting solid was filtered, washed with water (2 L) and dried to afford the desired product. LCMS: m/z=381.0 [M+H]; m/z=379.2 [M−H]. ¹H NMR (400 MHz, DMSO-d₆) δ 9.32 (bs, 1H), 8.55 (s, 1H), 8.22 (s, 1H), 8.10 (s, 1H), 7.54 (s, 1H), 3.60 (s, 3H), 2.27 (s, 3H), 1.49 (s, 9H).

Step 5: tert-butyl N-[6-[(6-cyano-4-methyl-3-pyridyl)oxy]-2-(methylamino)-3-nitro-4-pyridyl]carbamate (197 g, 0.518 mol, 1.0 eq) was suspended in a mixture of Hydrochloric acid, 4N solution in water (1 L) and THF (1 L). The reaction mixture was heated at 60° C. during 5 hours. The reaction mixture was cooled down to room temperature and the solid was filtered, washed with THF (1 L) and dried to afford the desired product as hydrochloric salt. LCMS: m/z=281.4 [M+H]. ¹H NMR (400 MHz, DMSO-d₆) δ ppm: 9.28 (s, 1H), 8.54 (s, 1H), 8.12 (s, 1H), 7.57 (bs, 2-3H), 6.33 (s, 1H), 3.67 (s, 3H), 2.25 (s, 3H).

### Compound 1: 4-Methyl-5-[3-methyl-7-(6-morpholin-4-yl-pyridazin-3-ylamino)-3H-imidazo[4,5-b]pyridin-5-yloxy]-pyridine-2-carbonitrile

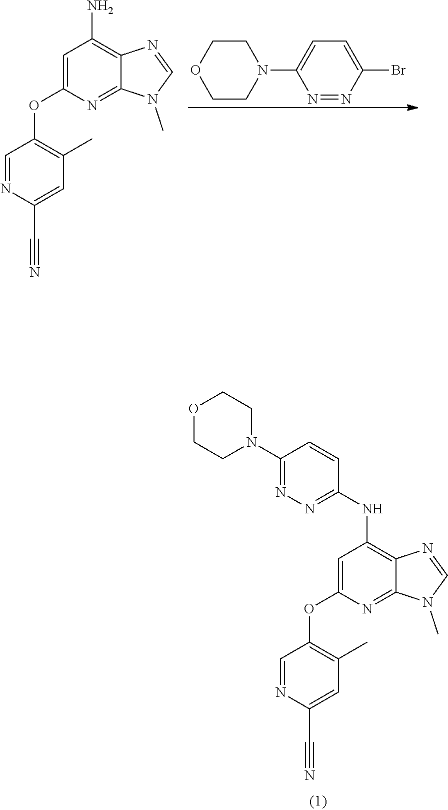

### Route 1

Intermediate 3 (1.0 eq, 409 g, 1.459 moles) and 4-(6-bromopyridazin-3-yl)morpholine (CAS [66346-91-6], 1.1 eq, 392 g) were added to xylene mix of isomers (8 L) at room temperature. To the mixture was added, under stirring at room temperature, potassium phosphate tribasic (3.0 eq, 929 g). The reaction mixture was heated from room temperature to 135° C. in 2 h30. Then a suspension of Pd(OAc)₂ (2 mol %, 6.6 g) and Xantphos (4 mol %, 33.8 g) in xylene (50 mL) was added to the hot mixture. The reaction was heated at reflux for 1 h30. Then a suspension of Pd(OAc)₂ (2 mol %, 6.6 g) and Xantphos (4 mol %, 33.8 g) in xylenes (50 mL) was added and the reaction was heated at reflux for an additional 1 h30. Then a suspension of Pd(OAc)₂ (2 mol %, 6.6 g) and Xantphos (4 mol %, 33.8 g) in xylenes (50 mL) was added one last time. The reaction was refluxed for an additional 1 h30. The reaction mixture was cooled down to room temperature and stirred overnight. The suspension was filtered, washed with acetonitrile (5 L). The solid was washed with water (15 L) until obtaining a neutral pH, dried under suction, then suspended in acetonitrile (6.5 L) and stirred at room temperature for 1 h. The suspension was filtered, washed with acetonitrile (2 L) and dried. Chromatography on SiO₂ (1 g of SiO₂ for 1 g of crude) using eluent CHCl₃/acetone (70/30) and then CHCl₃/MeOH (96/4) afforded the desired product (Compound 1).

### Route 2

Intermediate 3 (280 mg, 1 mmol, 1.0 eq), 4-(6-bromopyridazin-3-yl)morpholine (268 mg, 1.1 mmol, 1.1 eq) and CsCO₃ (977 mg, 3 mmol, 3 eq) are mixed under argon at room temperature and degassed tert-amyl alcohol or DMF (5 mL) is added. [Pd(cinnamyl)Cl]₂ (5.18 mg, 0.010 mmol, 0.01 eq) and JosiPhos (CAS[1702311-34-9]) (13 mg, 0.024 mmol, 0.024 eq) are added under argon either as solid or as pre-mixed solution in 1 mL degassed tert-amyl alcohol or DMF. The mixture is heated to 100° C. for at least 2 h.

The reaction mixture is then cooled to room temperature and acetonitrile is added. The suspension is filtered, the solid is triturated first with water then acetonitrile, and dried to afford the desired product (Compound 1).

### BIOLOGICAL EXAMPLES

The methods for the preparation of the Compound of the Invention and certain biological examples have been described in WO 2019/076716 (the Compound of the Invention being referred to as “compound 38” in WO 2019/076716).

Herewith are provided additional biological examples and data investigating compound 1 as a cytochrome P450 (CYP) and P-gp substrate.

### Example 1. In Vitro Stability Study of Compound 1 in Human Liver Microsomes

### 1.1. Study Objective

The objective of this in vitro study was to determine the stability of Compound 1 in human liver microsomes (HLM) and in human recombinant cytochrome P450 (CYP) enzymes. Following identification of CYP3A4 as the only CYP metabolising enzyme, the kinetic parameters Km (concentration of substrate at which half maximal rate of reaction is reached) and Vmax (maximum rate of reaction) parameters were determined using recombinant enzymes.

### 1.2. Materials and Methods

Compound 1 was incubated at 3 concentrations (0.1, 0.5 and 1 μM) at 37° C. with HLM (with NADPH) at 0.5 and 2 mg/mL for up to 45 minutes to determine whether sufficient metabolism was observed to warrant further investigation in the presence of specific chemical inhibitors.

Compound 1 (0.1 μM) was also incubated with human recombinant CYP enzymes (1A2, 2B6, 2C8, 2C9, 2C19, 2D6 and 3A4) and NADPH at 37° C. for up to 45 min to identify potential enzymes involved in the metabolism of Compound 1 and to determine conditions for further enzyme kinetics investigations.

Subsequently, Compound 1 was incubated with human CYP3A4 recombinant enzyme at 11 substrate concentrations (0.08-200 μM) in triplicate at 37° C. with NADPH in order to determine the depletion rate constant at each concentration and thus calculate the Km and Vmax values.

The stability of Compound 1 was assessed by the quantification of the concentration of Compound 1 in the samples using LC-MS/MS analytical method.

### 1.3. Results of the Study

Compound 1 showed low metabolism in HLM resulting in an intrinsic clearance up to 8.72 L/min/mg protein.

No measurable metabolism of Compound 1 was observed following incubation with recombinant CYP1A2, CYP2B6, CYP2C8, CYP2C9, CYP2C19 and CYP2D6. Metabolism of Compound 1 was observed following incubation with CYP3A4.

In the presence of CYP3A4 recombinant enzymes using the substrate depletion approach, the Km for Compound 1 was 4.19±0.414 μM and the calculated Vmax was 1.78 pmol/min/pmol P450 (577 pmol/min/mg microsomal protein).

In conclusion, Compound 1 is an in vitro substrate of the human CYP3A4 isoform.

### Example 2. In Vitro Stability Study of Compound 1 as P-Gp Substrate

### 2.1. Study Objective

The objective of these in vitro studies was to inform whether Compound 1 was a potential substrate of the human ABC (efflux) transporter MDR1 (P-gp) and then to determine the kinetic parameters Km and Vmax parameters of Compound 1 with human MDR1 transporter.

### 2.2. Materials and Methods

Compound 1 was incubated at 3 concentrations (0.625, 2.00 and 6.25 μM; triplicates) in bidirectional permeability assay (monolayer substrate assay) with Madin-Darby canine kidney multidrug resistance 1 (MDCKII-MDR1) transfected cells for 0, 60 and 120 minutes to determine whether there was active transport (direction apical to basolateral [A-B] and basolateral to apical [B-A]). As an active transport was identified, Compound 1 (2 pM; triplicates) was incubated for 120 minutes with MDCKII-MDR1 cells in the presence of a strong MDR1 inhibitor (10 μM valspodar) to confirm the specificity of the MDR1 transporter.

Subsequently, Compound 1 was incubated with MDCKII-MDR1 cells at 8 concentrations (0.50-6.0 μM) in triplicate for 15, 30, 60 and 120 minutes in order to determine the Km and Vmax values to test the unidirectional permeability B-A.

Bidirectional transport of Compound 1 was determined by quantification of Compound 1 in the samples using a LC-MS/MS analytical method.

### 2.3. Results of the Study

Compound 1 showed higher permeability in the B-A direction than in the A-B direction, indicating that there was active transport of this compound in the MDCKII-MDR1 cells. Considering the best recovery values (>60%) of Compound 1 at 6.25 μM, the observed net efflux ratio (net ER) was 34.6 after 60 minutes incubation. In the follow-up assay, the net ER decreased from 37.1 to 0.5 in the presence of valspodar, confirming the contribution of MDR1 to the transport of Compound 1 across MDCKII-MDR1 monolayers.

In the unidirectional permeability B-A, the net ER was consistent with the previous results. The kinetic parameters Km was 15.06 μM and Vmax was 8.79 pmol/min.

In conclusion, Compound 1 is an in vitro substrate of the human MDR1 ABC efflux transporters.

### CLINICAL EXAMPLES

TABLE II
List of abbreviations as used herein above and below:

| | |
|---|---|
| AE | adverse event |
| ALT | alanine aminotransferase |
| AST | aspartate aminotransferase |
| AUC | area under the plasma concentration-time curve |
| AUC_{0-24 h} | area under the plasma concentration-time curve |
| | from time zero till 24 hours postdose |
| AUC₀₋ₜ | area under the plasma concentration-time curve |
| | from time zero till the last observed |
| | quantifiable concentration |
| AUC_{0-∞} | area under the plasma concentration-time curve |
| | from time zero to infinity |
| AUC_{τ} | area under the plasma concentration-time curve |
| | over the dosing interval |
| BA | bioavailability |
| b.i.d. | twice daily |
| BMI | body mass index |
| BSA | body surface area |
| ° C. | celsius |
| CI | confidence interval |
| CKD-EPI | chronic kidney disease epidemiology collaboration |
| CL/F | apparent total plasma clearance, calculated as Dose/AUC_{0-∞} |
| Cₘₐₓ | maximum observed plasma concentration |
| C_{max, ss} | maximum observed plasma concentration at steady state |
| C_{T} | trough plasma concentration observed at the end |
| | of the dosing interval |
| CSP | clinical study protocol |
| CYP | cytochrome P450 |
| DDI | drug-drug interaction |
| DLQI | Dermatology Quality of Life Index |
| DMSO | Dimethylsulphoxide |
| ECG | electrocardiogram |
| eGFR | estimated glomerular filtration rate |
| EtOH | Ethanol |
| FACS | fluorescence-activated cell sorting |
| FDA | United States Food and Drug Administration |
| FE | food effect |
| FIH | first-in-human |
| FSH | follicle-stimulating hormone |
| FU | follow-up |
| g | gram |
| g′ | G force |
| GM-CSF | granulocyte-macrophage-colony stimulating factor |
| GMR | geometric mean ratio |
| h | hour |
| HBsAg | hepatitis B virus surface antigen |
| HCV | hepatitis C virus |
| HED | human equivalent dose |
| HIV | human immunodeficiency virus |
| hWBA | human whole blood assay |
| IB | investigator's brochure |
| ICF | informed consent form |
| IEC | Independent Ethics Committee |
| IFN | interferon |
| INFα | interferon alpha |
| IL | interleukin |
| IMP | investigational medicinal product |
| IP | investigational product |
| IRB | Institutional Review Board |
| JAK | Janus kinase |
| K_{I} | Concentration of inhibitor which gives half |
| | maximal rate of inactivation |
| K2EDTA | dipotassium ethylenediaminetetraacetic acid |
| kg | kilogram |
| L | liter |
| λz | apparent terminal elimination rate constant |
| LC | Liquid chromatography |
| m | meter |
| MAD | multiple ascending dose |
| MDZ | midazolam |
| | maximum recommended starting dose |
| mg | milligram |
| min | minute |
| mL | milliliter |
| MS/MS | Triple quadrupole mass spectrometry |
| NADPH | nicotinamide adenine dinucleotide phosphate |
| NC | non clinical |
| No. | number |
| OTC | over-the-counter |
| PASI | Psoriasis Area and Severity Index |
| PCR | polymerase chain reaction |
| PD | pharmacodynamic(s) |
| P-gp | permeability glycoprotein |
| PK | pharmacokinetic(s) |
| q.d. | quaque die; once daily |
| QT | measurement made on an electrocardiogram used to assess |
| | some of the electrical properties of the heart |
| QTcF | QT interval corrected for the heart rate using |
| | Fridericia's formula QTcF = QT/RR^{1/3} |
| | RR = the interval from the onset of one QRS |
| | complex to the onset of the next QRS complex |
| R_{ac} | accumulation ratio |
| rBA | relative bioavailability |
| RT-PCR | real-time polymerase chain reaction |
| SAD | single ascending dose |
| SAE | serious adverse event |
| SARS-CoV-2 | severe acute respiratory syndrome coronavirus 2 |
| SD | standard deviation |
| sPGA | static Physician's Global Assessment |
| (p)STAT | (phosphorylated) signal transducer and |
| | activator of transcription |
| Th | T helper |
| t_{1/2} | elimination half-life |
| tₘₐₓ | time of occurrence of Cₘₐₓ |
| t_{1/2, λ, z} | terminal elimination half-life, calculated from (In 2)/λz |
| TEAE | treatment-emergent adverse event |
| TYK | tyrosine kinase |
| TYK2 | tyrosine kinase 2 |
| ULN | upper limit of normal range |
| Vd/F | apparent total volume of distribution, |
| | calculated as Dose/λz * AUC0-∞ (single dose) |
| WBA | whole blood assay |
| WOCBP | woman of childbearing potential |

### Example 1. First-In-Human Clinical Study

The study of the current example was a first-in-human (FIH) randomized, double-blind, placebo-controlled study to evaluate the safety, tolerability, and pharmacokinetics of single and multiple ascending oral doses of Compound 1 in adult, healthy, male subjects.

The primary objective of this study was to evaluate the safety and tolerability of single and multiple ascending oral doses of Compound 1 in adult, healthy, male subjects compared with placebo.

### 1.1. Study Endpoints

### Primary Outcome Measure was:

To evaluate the frequency and severity of treatment-emergent adverse event (TEAEs), treatment-emergent serious adverse events (SAEs), and TEAEs leading to treatment discontinuations in adult, healthy, male subjects.
   Secondary Outcome Measures were:
PK parameters of Compound 1 in plasma of adult, healthy, male subjects: Cₘₐₓ, AUC, t_{1/2}.
   Other Outcome Measures were:
Cₘₐₓ and AUC following single oral dose of Compound 1 as a suspension, in fed and fasted conditions, in adult, healthy, male subjects.
Cₘₐₓ and AUC following single oral dose of Compound 1 as a suspension and as capsules, in fed conditions, in adult, healthy, male subjects.
Cₘₐₓ and AUC following single oral dose of Compound 1 as capsules, in fed and fasted conditions, in adult, healthy, male subjects.
Phosphorylated signal transducer and activator of transcription (pSTAT) inhibition following ex vivo cytokine stimulation of whole blood cells measured by flow cytometry.
Gene expression in whole blood, serum levels of neopterin and β2-microglobulin, heart rate and body temperature after in vivo IFN-α challenge.
Ratio of endogenous biomarkers before and after administration with Compound 1.

### 1.2. Number of Subjects

Up to 60 subjects were planned to be enrolled; i.e. 16 subjects in Part 1 (SAD), 24 subjects in Part 2 (MAD), 8 subjects in Part 3 (FE), and 12 subjects in Part 4 (rBA/FE). In total, 52 subjects were enrolled in the study.

### 1.3. Inclusion Criteria

To be eligible, the subjects met the following criteria:
Male between 18 and 55 years of age (extremes included), on the date of signing the ICF.
A body mass index (BMI) between 18 and 30 kg/m2, inclusive.
Judged to be in good health by the investigator based upon the results of a medical history, physical examination, vital signs, 12-lead ECG, and fasting clinical laboratory safety tests, available at screening and prior to randomization. Hemoglobin, neutrophil, lymphocyte, and platelet counts had to be above the lower limit of normal range. Bilirubin, aspartate aminotransferase, and alanine aminotransferase (ALT) had to be within normal ranges. Other clinical laboratory safety test results had to be within the reference ranges or test results that were outside the reference ranges needed to be considered not clinically significant in the opinion of the investigator.

Key exclusion criteria: subjects meeting one or more of the following criteria were not selected for this study:
Known hypersensitivity to investigational product (IP) ingredients or history of a significant allergic reaction to IP ingredients as determined by the investigator.
Known contraindication or hypersensitivity to IFN-α or any component of Intron-A® (Note: this criterion was only applicable to subjects in the MAD part).
Positive serology for hepatitis B virus surface antigen or hepatitis C virus (HCV) or history of hepatitis from any cause with the exception of hepatitis A that was resolved at least 3 months prior to first dosing of the IP.

### 1.4. Study Design/Methodology

This was a first-in-human study consisting of 4 Parts.
Part 1 (single ascending doses [SAD]) was randomized, double-blind, placebo-controlled, and included 6 sequential dose levels in 2 alternating cohorts of 8 subjects each (Cohorts A and B) resulting in a total of 16 subjects. Subjects in fasted (dose levels 1 to 4) or fed (dose levels 5 and 6) state received a maximum of 3 single doses of Compound 1 or placebo. Each subject in Part 1 was in the study for approximately 9 weeks (from screening to follow-up [FU] visit).
Part 2 (multiple ascending doses [MAD]) was randomized, double-blind, placebo-controlled, and included 3 sequential cohorts of 8 subjects each (Cohorts C, D, and E) resulting in a total of 24 subjects. Subjects in fed state received Compound 1 or placebo once daily (q.d.) for 13 days. On Day 11, subjects received an interferon (IFN)-α challenge (i.e. a single subcutaneous injection of 1 million IU of Intron A® in the abdomen) within 30 minutes after the Compound 1/placebo administration. Each subject in Part 2 was in the study for approximately 7 weeks (from screening to FU visit).
Part 3 (food effect [FE]) on the suspension was not performed.
Part 4 (relative bioavailability [rBA]/FE) consisted of 1 cohort (Cohort I) of 12 subjects in a randomized, open-label, crossover design. Subjects in fed state received Compound 1 once as an oral suspension and once as capsules for oral use. In addition, the exposure of Compound 1 administered as oral capsules was evaluated under fasted conditions. Each subject in Part 4 was in the study for approximately 6 weeks (from screening to FU visit).

In Parts 1 and 2, subjects were randomized in a 3:1 ratio to Compound 1 or placebo. In Part 4, subjects were randomized in a 1:1:1:1:1:1 ratio, to 1 of 6 treatment sequences.

Safety assessments were performed throughout the study and included assessment of adverse events (AEs), clinical laboratory safety parameters, vital signs, physical examinations, and 12-lead electrocardiograms (ECGs).

Blood (all Parts) and urine (only Parts 1 and 2) samples were collected for the determination of Compound 1 in plasma and urine and selected PK parameters were assessed.

### 1.5. Test Product, Control Product, Dose, Mode of Administration

Compound 1 was provided as an oral suspension (Parts 1, 2, and 4) or as a capsule for oral use (Part 4 only). Before administration, the oral suspension was prepared by the clinical center pharmacist by dispersing amorphous solid dispersion powder in a vehicle for oral use.

The starting dose was set to 10 mg Compound 1. Next dose levels were defined based on emerging safety/tolerability and PK data during the study. The dose levels that were actually administered are given below:
Part 1 (SAD): 10 mg (fasted) in Cohort A study period 1, 30 mg (fasted) in Cohort B study period 1, 90 mg in Cohort A study periods 2 (fasted) and 3 (fed), 200 mg in Cohort B study periods 2 (fasted) and 3 (fed).
Part 2 (MAD) (fed): 30 mg q.d. in Cohort C, 90 mg q.d. in Cohort D, 150 mg q.d. in Cohort E.
Part 4 (rBA/FE): 150 mg in Cohort I (suspension [fed] and capsules [fed and fasted]).

Placebo was the control product for Parts 1 and 2 and was provided as an oral suspension.

### 1.6. Duration of Treatment

In Part 1, subjects received a single dose of Compound 1 or placebo on Day 1 of each study period, with a washout period of 14 days. In Part 2, subjects received Compound 1 or placebo q.d. from Day 1 to Day 13. In Part 4, subjects received a single dose of Compound 1 during 3 treatment periods, with a washout period of 4 days.

### 1.7. Safety Endpoints

AEs were coded according to the Medical Dictionary for Regulatory Activities version 23.0. An analysis of the TEAEs was performed. For clinical laboratory evaluations, vital signs, and 12-lead ECGs, descriptive statistics were used to summarize the actual values and the changes from baseline per scheduled time point and treatment group. Tabulations of the shifts from baseline according to normal ranges, were provided per treatment group for worst-case and treatment-emergent worst-case. Physical examination abnormalities postbaseline were listed. The safety assessments as of the IFN-α administration on Day 11 in MAD part (Part 2) were analyzed separately.

### 1.8. Pharmacokinetic Assessment

Descriptive statistics were calculated by treatment group (Part 1—SAD) and by treatment group and day (Part 2—MAD) for the plasma concentrations, urine amounts (if applicable), and on the listed PK parameters. Mean (±standard deviation) plasma concentrations of Compound 1 versus time were plotted per treatment group (Part 1—SAD) and per treatment group and day (Part 2—MAD).
In Part 1, dose proportionality of Compound 1 was tested using a power model approach. A mixed-effects model was applied to the log-transformed PK parameters (including Cₘₐₓ and AUC_{0-∞}) of Compound 1. Dose effect on time of occurrence of Cₘₐₓ (tₘₐₓ) was assessed using a non-parametric Kruskal-Wallis test.
In Part 2, dose proportionality of Compound 1 was tested using a power model approach. A mixed-effects model was applied to the log-transformed PK parameters (including Cₘₐₓ and AUC_{0-∞}) of Compound 1. Dose effect on tₘₐₓ was assessed using a non-parametric Kruskal-Wallis test. Time to reach steady state was assessed by visual inspection of the trough plasma concentrations of Compound 1.

### 1.9. Pharmacokinetic Measurements

Blood samples for determination of Compound 1 in plasma were collected on the visit. In Part 1, blood samples for determination of coproporphyrins I and III in plasma were collected on the visits. In Part 2, blood samples for 4-β-OH-cholesterol and cholesterol determination in plasma were collected before Compound 1/placebo administration on the visits. All blood samples were collected by venipuncture (or indwelling canula) preferably in the forearm.

Samples for the determination of Compound 1 in urine were collected in Parts 1 and 2 on the visits. Urine samples in Part 1 were also used for the determination of thiamine. Urine samples were collected by interval (related to dosing, if needed subjects could be requested to void/empty their bladder to have a more accurate relation between urine production and collection).

Determination of Compound 1 in urine was performed on the samples from the MAD cohort subjects who were administered the highest dose. Based on the percentage of the dose excreted unchanged in urine in this cohort, it was decided to not quantify Compound 1 in urine samples from the other cohorts of the MAD part and SAD part.

### 1.10. Pharmacokinetic Results

In the SAD part, subjects received a single dose of Compound 1 in fasted state (10, 30, 90, or 200 mg) or fed state (90 or 200 mg). The arithmetic mean (coefficient of variation [CV] in %) of the main PK parameters of Compound 1 for Part 1 (SAD) are summarized in Table I below. Single dosing of Compound 1 showed a dose proportional increase in exposure in fasted state, with no impact of food up to 90 mg. Higher exposures were achieved under fed conditions with linear increase in exposure up to 200 mg.

TABLE I

| | Compound 1 | | | | | |
|---|---|---|---|---|---|---|
| | 10 mg, | 30 mg, | 90 mg, | 90 mg, | 200 mg, | 200 mg, |
| | fasted | fasted | fasted | fed | fasted | fed |
| PK Parameter | (N = 6) | (N = 6) | (N = 6) | (N = 5) | (N = 6) | (N = 6) |
| Cₘₐₓ | 0.117 | 0.248 | 0.669 | 0.826 | 0.457 | 1.42 |
| (μg/mL) | (16.8) | (45.8) | (30.6) | (23.7) | (50.9) | (11.6) |
| tₘₐₓ | 1.50 | 1.75 | 2.00 | 2.00 | 1.75 | 3.00 |
| (h) | (1.00-2.00) | (1.00-4.00) | (1.00-4.00) | (1.00-4.00) | (0.50-4.00) | (1.50-6.00) |
| AUC_{0-∞} | 1.60 | 3.62 | 10.9 | 11.1 | 7.00 | 25.1 |
| (μg · h/mL) | (52.9) | (56.5) | (49.9) | (51.4) | (55.9) | (40.4) |
| t_{1/2} | 10.8 | 12.0 | 11.3 | 9.53 | 12.3 | 12.3 |
| (h) | (39.2) | (28.5) | (43.2) | (43.2) | (22.1) | (26.2) |

AUC_{0-∞} = area under the plasma concentration-time curve from time zero to infinity;
N = number of subjects.
Values are arithmetic mean (CV %) except for tₘₐₓ: median (min-max).

After a single oral dose of 10 mg to 200 mg, in fasted or fed state (following a standard breakfast), Compound 1 was quantifiable from the first sampling time point, i.e. 0.5 hours postdose, in plasma of all subjects. Subsequently, mean plasma concentrations increased quickly, reaching a maximum at time points ranging from 1 to 4 hours postdose across doses. All dose levels showed a bi-phasic decline in mean plasma concentrations with a rapid initial distribution/elimination phase followed by a slightly slower terminal elimination phase.

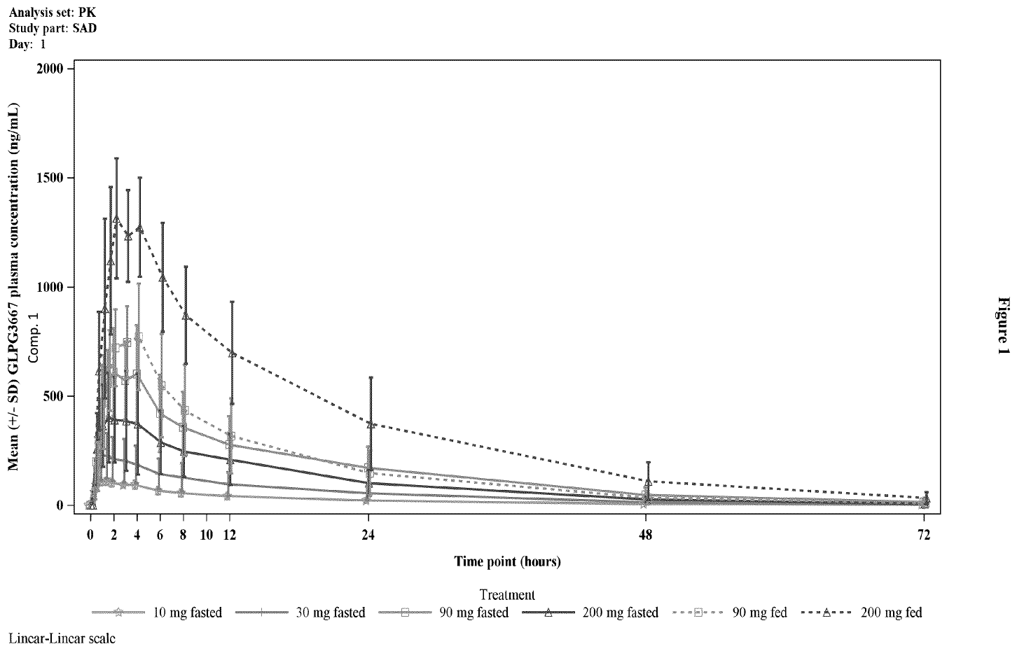

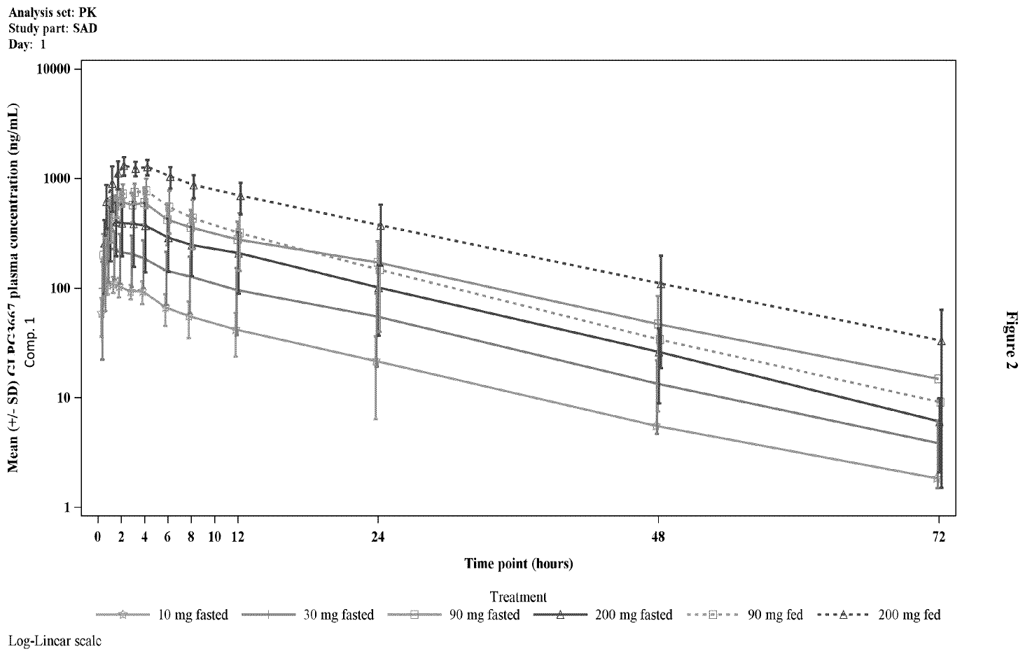

In the lowest dose group (10 mg Compound 1), Compound 1 plasma levels remained quantifiable until the last sampling time point (i.e. 72 hours) for 3 out of 6 subjects. In all other treatment groups, Compound 1 plasma levels remained quantifiable until the last sampling time point (i.e. 72 hours), with the exception of one subject in the 30-mg and 90-mg (fed and fasted) Compound 1 groups (FIGS. 1 and 2).

Following a single oral administration in adult, healthy male subjects in fasted state, Compound 1 was quickly absorbed with median t. ranging from 1.5 to 2 hours. Following administration in fed state, median tₘₐₓ ranged from 2 to 3 hours (Table II).

The mean t_{1/2} of Compound 1 was similar across all dose levels, ranging from 9.53 to 12.3 hours (Table II).

TABLE II

| | Compound 1 | Compound 1 | Compound 1 | Compound 1 | Compound 1 | Compound 1 |
|---|---|---|---|---|---|---|
| | 10 mg, | 30 mg, | 90 mg, | 90 mg, | 200 mg, | 200 mg, |
| | fasted | fasted | fasted | fed | fasted | fed |
| PK Parameter | N = 6 | N = 6 | N = 6 | N = 5 | N = 6 | N = 6 |
| Cₘₐₓ | 0.117 | 0.248 | 0.669 | 0.826 | 0.457 | 1.42 |
| (μg/mL) | (16.8) | (45.8) | (30.6) | (23.7) | (50.9) | (11.6) |
| tₘₐₓ | 1.50 | 1.75 | 2.00 | 2.00 | 1.75 | 3.00 |
| (h) | (1.00-2.00) | (1.00-4.00) | (1.00-4.00) | (1.00-4.00) | (0.50-4.00) | (1.50-6.00) |
| AUC_{0-24 h} | 1.19 | 2.66 | 7.61 | 8.70 | 5.24 | 17.7 |
| (μg · h/mL) | (36.0) | (54.5) | (42.4) | (42.9) | (54.9) | (26.7) |
| AUC₀₋ₜ | 1.54 | 3.54 | 10.5 | 11.0 | 6.89 | 24.4 |
| (μg · h/mL) | (51.3) | (57.1) | (48.2) | (50.7) | (56.1) | (38.7) |
| AUC_{0-∞} | 1.60 | 3.62 | 10.9 | 11.1 | 7.00 | 25.1 |
| (μg · h/mL) | (52.9) | (56.5) | (49.9) | (51.4) | (55.9) | (40.4) |
| t_{1/2} | 10.8 | 12.0 | 11.3 | 9.53 | 12.3 | 12.3 |
| (h) | (39.2) | (28.5) | (43.2) | (43.2) | (22.1) | (26.2) |
| Cₘₐₓ/dose | 11.7 | 8.27 | 7.43 | 9.18 | 2.28 | 7.08 |
| (ng/mL/mg) | (16.8) | (45.8) | (30.6) | (23.7) | (50.9) | (11.6) |
| AUC_{0-∞}/dose | 160 | 121 | 121 | 124 | 35.0 | 125 |
| (ng · h/mL/mg) | (52.9) | (56.5) | (49.9) | (51.4) | (55.9) | (40.4) |

N = number of subjects.
Values are arithmetic means (CV %) except for tₘₐₓ: median (min-max).

In the MAD part, the PK was assessed in subjects who received multiple doses of Compound 1 q.d. (30, 90, or 150 mg) for 13 days in fed state. The arithmetic mean (CV %) of the main PK parameters of Compound 1 for Part 2 (MAD) are summarized in the table below. There was no pronounced deviation from dose proportionality at steady state.

TABLE III

| | Compound 1 | | | | | |
|---|---|---|---|---|---|---|
| | 30 mg q.d., | | 90 mg q.d., | | 150 mg q.d., | |
| | fed | | fed | | fed | |
| | (N = 6) | | (N = 6) | | (N = 6) | |
| PK Parameter | Day 1 | Day 10 | Day 1 | Day 10 | Day 1 | Day 10 |
| Cₘₐₓ | 0.367 | 0.468 | 0.973 | 1.13 | 1.29 | 1.68 |
| (μg/mL) | (13.4) | (23.0) | (10.9) | (17.3) | (21.7) | (19.9) |
| tₘₐₓ | 3.00 | 3.50 | 3.00 | 3.00 | 2.00 | 3.50 |
| (h) | (2.00-4.00) | (2.00-4.00) | (2.00-4.00) | (1.50-4.00) | (1.50-4.00) | (3.00-4.00) |
| AUC_{0-τ} | 4.43 | 5.87 | 11.6 | 15.4 | 15.8 | 22.4 |
| (μg · h/mL) | (19.3) | (24.7) | (22.3) | (21.5) | (30.8) | (25.9) |
| t_{1/2} | NA | 14.5 | NA | 14.5 | NA | 13.2 |
| (h)^{a} | | (36.4) | | (12.3) | | (11.5) |

AUC_{0-τ} = area under the plasma concentration-time curve over the dosing interval; N = number of subjects; NA = not applicable.
^{a}t_{1/2} were estimated following last dosing on Day 13.
Values are arithmetic mean (CV %) except for tₘₐₓ: median (min-max).

After multiple oral doses of 30, 90, or 150 mg q.d., in fed state (following a standard breakfast), Compound 1 plasma concentrations were quantifiable from the first sampling time on Day 1 (0.5 hours postdose) and remained quantifiable until the last sampling time point (i.e. Day 16, 72 hours after last dosing) for all subjects.

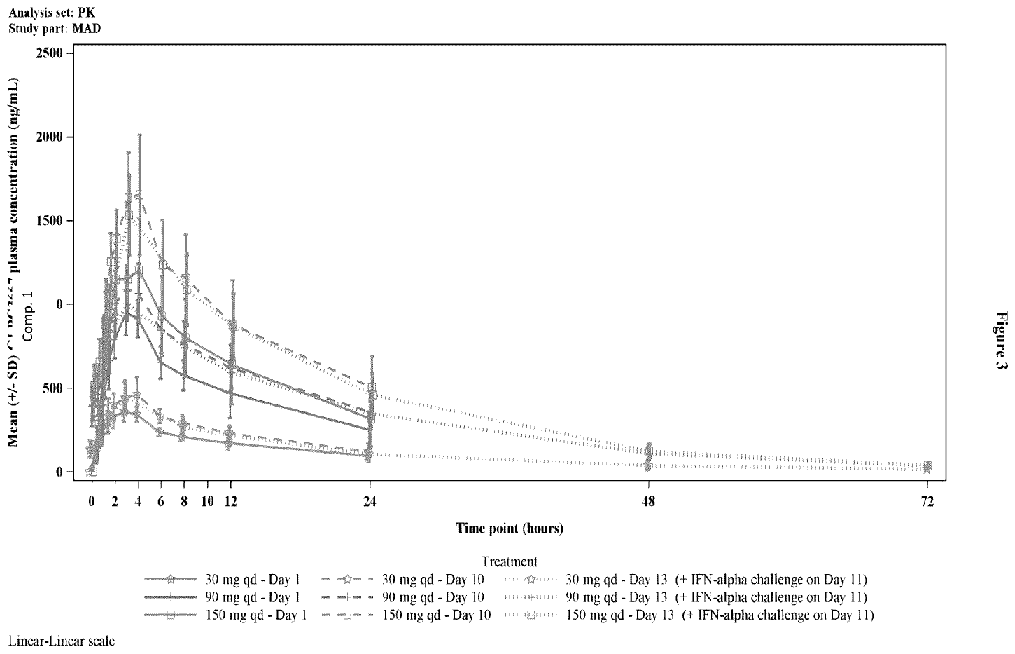

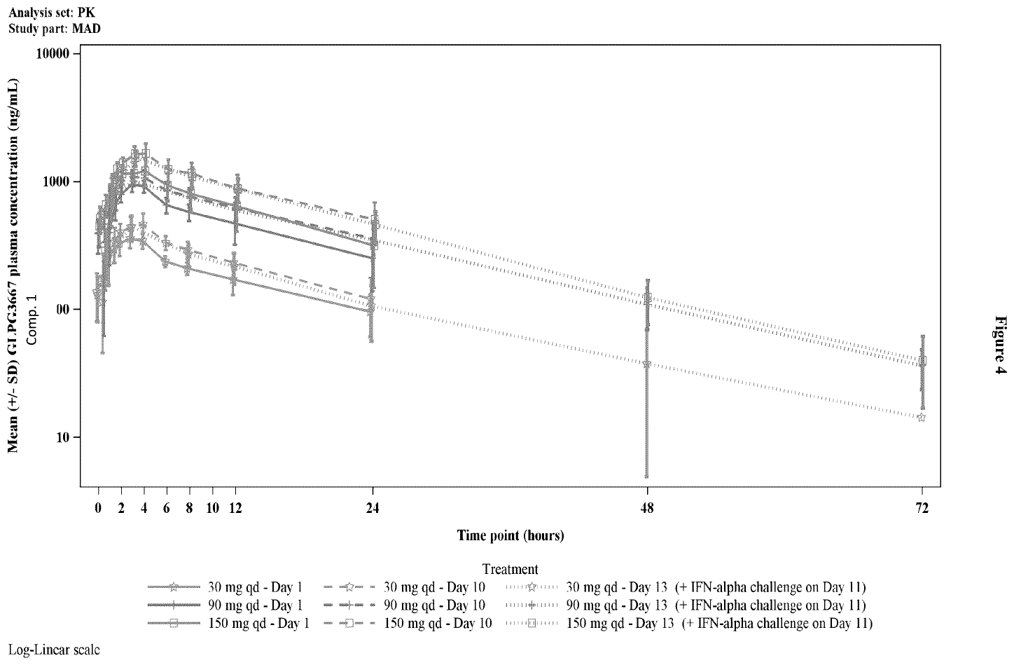

After a single dose on Day 1 or multiple doses on Day 10, mean plasma concentrations reached a maximum at time points ranging from 3 hours to 4 hours (FIGS. 3 and 4).

In line with the results after single dose, all dose levels showed a bi-phasic decline in meanplasma Compound 1 concentrations with a rapid initial distribution/elimination phase, followed by a slower terminal elimination phase (FIGS. 3 and 4).

Compound 1 was rapidly absorbed following single and repeated dosing under fed conditions at 30, 90, and 150 mg q.d. with median tₘₐₓ ranging from 2 to 3.5 hours, and remained similar throughout dosing (Table IV).

Mean t_{1/2} on the last dosing day (Day 13) was similar for all 3 dose levels, ranging from 13.2 to 14.5 hours and did not markedly deviate from results after a single dose (Table IV).

A moderate mean degree of accumulation to steady-state levels (R_{ac},) on Day 10 of 1.32, 1.33, and 1.46 was observed for the 30, 90, and 150 mg q.d. dose levels, respectively. This observation is consistent with estimated t_{1/2}, suggesting no marked time-dependent effect (Table IV).

TABLE IV

| | Compound 1 | | Compound 1 | | Compound 1 | |
|---|---|---|---|---|---|---|
| | 30 mg q.d., | | 90 mg q.d., | | 150 mg q.d., | |
| | fed | | fed | | fed | |
| | N = 6 | | N = 6 | | N = 6 | |
| PK Parameter | Day 1 | Day 10 | Day 1 | Day 10 | Day 1 | Day 10 |
| Cₘₐₓ | 0.367 | 0.468 | 0.973 | 1.13 | 1.29 | 1.68 |
| (μg/mL) | (13.4) | (23.0) | (10.9) | (17.3) | (21.7) | (19.9) |
| tₘₐₓ | 3.00 | 3.50 | 3.00 | 3.00 | 2.00 | 3.50 |
| (h) | (2.00-4.00) | (2.00-4.00) | (2.00-4.00) | (1.50-4.00) | (1.50-4.00) | (3.00-4.00) |
| C_{avg} | 0.185 | 0.244 | 0.484 | 0.642 | 0.656 | 0.933 |
| (μg/mL) | (19.3) | (24.7) | (22.3) | (21.5) | (30.8) | (25.9) |
| AUC_{0-τ} | 4.43 | 5.87 | 11.6 | 15.4 | 15.8 | 22.4 |
| (μg · h/mL) | (19.3) | (24.7) | (22.3) | (21.5) | (30.8) | (25.9) |
| AUC₀₋ₜ | NA | 7.62 | NA | 21.3 | NA | 29.1 |
| (μg · h/mL)¹ | | (37.8) | | (20.7) | | (24.5) |
| AUC_{0-∞} | NA | 8.02 | NA | 22.1 | NA | 29.9 |
| (μg · h/mL)¹ | | (42.6) | | (21.1) | | (25.6) |
| t_{1/2} | NA | 14.5 | NA | 14.5 | NA | 13.2 |
| (h)^{a} | | (36.4) | | (12.3) | | (11.5) |
| R_{ac} | NA | 1.32 | NA | 1.33 | NA | 1.46 |
| (AUC_{0-τ}) | | (10.8) | | (7.38) | | (15.7) |
| Cₘₐₓ/dose | 12.2 | 15.6 | 10.8 | 12.6 | 8.57 | 11.2 |
| (ng/mL/mg) | (13.4) | (23.0) | (10.9) | (17.3) | (21.7) | (19.9) |
| AUC_{0-τ}/dose | 148 | 196 | 129 | 171 | 105 | 149 |
| (ng · h/mL/mg) | (19.3) | (24.7) | (22.3) | (21.5) | (30.8) | (25.9) |
| AUC_{0-∞}/dose | NA | 267 | NA | 246 | NA | 199 |
| (ng · h/mL/mg)¹ | | (42.6) | | (21.1) | | (25.6) |
| Ae_{0-24 h} | NA | NA | NA | NA | 3.52 | 6.10 |
| (% dose) | | | | | (33.7) | (17.5)ⁿ⁼⁵ |
| CL_{R} | NA | NA | NA | NA | 0.360 | 0.432 |
| (L/h) | | | | | (42.1) | (48.2)ⁿ⁼⁵ |

N = number of subjects; n = number of subjects with data; NA = not applicable.
¹The PK parameters AUC₀₋ₜ, AUC_{0-∞}, t_{1/2}, and AUC_{0-∞}/dose were estimated on Day 13.
Values are arithmetic means (CV %) except for tₘₐₓ: median (min-max).

Compound 1 exposure (Cₘₐₓ and AUC_{0-∞}) was similar following a single oral administration of Compound 1 as capsules or suspension under fed conditions (Table V).

TABLE V

| | Compound 1 | Compound 1 | Compound 1 |
|---|---|---|---|
| | susp. fed | caps. fasted | caps. fed |
| PK Parameter | N = 12 | N = 12 | N = 12 |
| Cₘₐₓ (μg/mL) | 1.19 (20.6) | 0.676 (26.6) | 1.17 (15.7) |
| tₘₐₓ (h) | 4.00 (2.00-4.00) | 2.50 (1.50-4.00) | 3.50 (2.00-4.00) |
| AUC_{0-24 h} (μg · h/mL) | 15.4 (25.9) | 8.59 (31.5) | 14.5 (19.5) |
| AUC₀₋ₜ (μg · h/mL) | 22.2 (35.8) | 12.4 (42.4) | 20.8 (26.6) |
| AUC_{0-∞} (μg · h/mL) | 23.2 (39.1) | 13.1 (45.3) | 21.6 (28.0) |
| t_{1/2} (h) | 13.6 (30.1) | 14.9 (25.9) | 13.8 (20.7) |

caps = capsule; N = number of subjects; susp = suspension.
Values are arithmetic means (CV %) except for tₘₐₓ: median (min-max).

This was statistically confirmed with the 90% CIs of the least-squares geometric means ratio for Cₘₐₓ (85.92; 115.19) and for AUC_{0-∞} (83.65; 110.06), falling entirely within the bioequivalence range of 80% to 125% (Table VI).

TABLE VI

| PK Parameter | Treatment Pair | Pairwise Comparison¹ |
|---|---|---|
| Cₘₐₓ | capsule fed vs. | 99.48 (85.92; 115.19) |
| | suspension fed | 175.93 (151.95; 203.70) |
| | capsule fed vs. | |
| | capsule fasted | |
| AUC_{0-∞} | capsule fed vs. | 95.95 (83.65; 110.06) |
| | suspension fed | 171.66 (149.65; 196.90) |
| | capsule fed vs. | |
| | capsule fasted | |

¹Point estimate and 90% CI of the least-squares geometric means ratio.

Similar to the suspension, higher exposures were observed when Compound 1 capsules were administered under fed conditions with approximately 1.7- to 1.8-fold higher AUC_{0-∞} and Cₘₐₓ when compared to fasted state, respectively.

Median tₘₐₓ was comparable following administration of Compound 1 as suspension (4 hours) and capsules (3.5 hours) in fed state, while slightly lower following administration of Compound 1 as capsules in fasted state (2.5 hours).

Mean t_{1/2} remained similar in all treatment groups ranging from 13.6 to 14.9 hours.

### 1.11. Pharmacodynamic Measurements

Blood samples for evaluation of biomarkers were collected in Parts 1 and 2 by venipuncture (or indwelling cannula) preferably in the forearm on the visits. PD samples were to be collected after PK samples.

PD samples were collected at several time points coinciding with PK sampling to allow for PK/PD evaluations.

Selectivity and potency of Compound 1 were evaluated ex vivo by cytokine stimulation of whole blood cells, by measuring the level of phosphorylation of STATs (the direct target of JAKs). The following assays were performed using flow cytometry (fluorescence-activated cell sorting [FACS]):
IL-6-induced pSTAT1 (JAK1 inhibition measurement)
GM-CSF-induced pSTAT5 (JAK2 inhibition measurement)
IFN-α-induced pSTAT1 (JAK1/TYK2 inhibition measurement)
IL-12-induced pSTAT4 (JAK2/TYK2 inhibition measurement)

The same assays were implemented during the MAD part on Day 1, Day 10, and Day 16 to confirm the data collected during the SAD part. The assays on Day 10 provided potency and level of selectivity of the IP at steady state.

Individual IC₅₀ values were determined in vitro in an hWBA by measuring the inhibition of IL-6-induced pSTAT1, GM-CSF-induced pSTAT5, IFN-α-induced pSTAT1, and IL-12-induced pSTAT4 performed on Day −1 in the SAD (only for study period 1) and MAD parts.

The in vivo IFN-α challenge (on Day 11 of the MAD part) was assessed by measurement of IFN-α-induced biomarkers (neopterin, β2-microglobulin, body temperature, and heart rate). In addition, a gene expression analysis was performed on the PAXgene tube samples to evaluate the impact of Compound 1 on the IFN-α gene signature. A full genome transcriptomic analysis with an RNA-sequencing study was performed on the same samples if the gene expression analysis data on a selected relevant panel of genes demonstrate relevant impact of Compound 1 on the IFN-α gene signature.

1.12. FACS Analysis for pSTAT Analysis

### Blood Collection:

3 mL blood samples were collected into Lithium Heparin vacutubes (BD Vacutainer Heparin tubes reference 366667)
Samples were mixed well by inverting the tubes 3-4 times
Samples were then left on a bench rocker for 30 minutes at room temperature to equilibrate

### Blood Triggering:

Distributed 10 μL of IL-6 in 2 mL round bottom eppendorf-like tubes in duplicates (polypropylene).
Added 190 μL of blood in each reaction tube and lock the tube
Incubated at +37° C. for 20 minutes (60 minutes for IL-12) under gentle agitation on an orbital shaker

### Sample Preparation for Flow Cytometry Analysis

After the incubation period, per sample, transfered the 200 μL of incubated blood into one 5 mL round bottom tubes with cap (compatible for flow cytometry, polystyrene) by pipetting 200 μL with a P200 Gilson pipette
Added 4 mL of pre-warmed 1× Lyse/Fix buffer per tube
Incubated the tubes at +37° C. in a water bath for 10 minutes
Pelleted the cells by centrifugation at 500×g for 8 minutes, at room temperature
Discarded the supernatant by inverting the tubes (around 100 μL will be left)
Vortexed quickly the remaining volume to re-suspend the pellet
Re-suspended the pellet in 4 mL of PBS, transfer 2 mL in 1 round bottom tubes with cap labeled A and the tube with the remaining is labeled B (cf. label of the tubes)
Re-centrifuged the tubes at 500× g for 8 minutes at room temperature
Completely discarded the supernatant by inverting tube and blot it against clean paper towels
Added drop by drop 1 mL of ice-cold Perm Buffer III under gentle agitation on a vortex in each tube
Locked totally (2nd position of the cap) the tubes and immediately froze (within 5 minutes) them at approximately −20° C. until shipment

### Flow Cytometry Analysis

On the day of analysis cells in methanol were pelleted by centrifugation for 8 minutes at 500×g at room temperature and supernatant was removed by inverting the tubes. 2 mL of PBS+3% BSA buffer was added to each sample tube and vortexed briefly. Cells were pelleted by centrifugation for 8 minutes at 500×g at room temperature and supernatant was removed by inverting the tubes (around 100 μL were left). Cells were transferred to 96-well plate (BD Falcon, CatNo. 357711), and appropriate volume for the staining of different pSTAT and CD for different cell types was added to each well:
   Panel 1 (trigger groups of IFNα or IL-6): 20 μL of CD4 APC, 5 μL of pSTAT1 PE
   Panel 2 (trigger group of GM-CSF): 5 μL of CD33 APC, 20 μL of pSTAT5 PE
   Panel 3 (trigger group of IL-12): 20 μL of CD335 PE, 20 μL of pSTAT4 AF647
   Panel 4 (trigger group of IL-2): 5 μL of CD4 V450, 20 μL of pSTAT5 PE
Following staining, samples were briefly vortexed and incubated at room temperature in the dark for at least 1 hour. At the end of incubation, samples were washed with PBS by adding 150 μL of PBS to each well, pelleted by centrifugation for 8 minutes at 500×g at room temperature and supernatant was removed by flicking the plates. Washing step was repeated by addition of 230 μL of PBS to each well, plate was centrifuged for 8 minutes at 500×g at room temperature and supernatant was removed by flicking the plates. After the last washing step, 240 μL of PBS was added to each well and resuspended by pipetting. Half of the suspension, 120 μL, from each well was transferred to a new plate as a technical duplicate. 120 μL of PBS was added to each well of each plate and samples were resuspended by pipetting. Before the analytical run was started, untriggered and triggered controls from different patients were used to check the PMT voltage of each detector in order to confirm that the staining was done properly and to adjust the voltages if necessary. One of the plates was used immediately for analysis at Attune N×T cytometer. The other plate was stored at 4° C. until the end of the analysis and would be analyzed the same day if needed.
Although the same analytical method was used for the analysis of all samples, separate analytical runs were used for the analysis of different cell types. Regardless of the cell type, each analytical run contained patient and blank samples.
Each run consisted of:
   64-80 study samples from 8 patients (8-10 samples per patient). Each sample was described with Dummy ID for each subject of each cohort (provided by Fidelta d.o.o. and containing 2 letters and one number) and aliquot name (provided by SGS, containing 5 letters or 4 letters and one number).
   One blank sample (DPBS only) was placed after eight samples from the same patient and was used as a washout between different patients for Cohorts A1, B1, A2, B2, A3 and B3. These samples were marked as _E.
   Two blank samples (DPBS only) were placed after 7th and 9th sample (between day −1 and day 2 samples), or after 7th and 10th sample (between day 10 and day 16 samples) from the same patient and were used as a washout between unstimulated and stimulated sample from the same patient and between different patients for Cohorts Cl, D1 and E1. These samples were marked as _E.
Gating Strategy
   Flow cytometry data analysis is fundamentally based upon the principle of gating. Gates and regions are placed around populations of cells with common characteristics, usually forward scatter, side scatter and marker expression, to investigate and to quantify these populations of interest.
   For each cell type that was analyzed, similar gating strategy was done. First gate was done for doublet exclusion (named Single cells) followed by two gates were two-parameter density plots named P1 and P2. These plot display two measurement parameters, one on the x-axis and one on the y-axis and the events as a density plot.
   P1 gate was based on forward (FSC) and side (SSC) scatter that give an estimation of the size and granularity of the cells respectively.
   P2 gate was based on SSC and antibody that was used to stain cell population of interest. Each antibody was specific for different cell population (CD4 APC for CD4 T lymphocytes, CD33 for monocytes and CD335 for natural killer cells).
   Single parameter histogram was used for setting P3 gate. There are usually two peaks which can be interpreted as the positive and negative dataset. Untriggered control was used for setting the P3 gate with less than 1% positive cells gated unless the high background phosphorylation of Stat protein of interest was detected or the trigger control was not stimulated properly. This gate was dragged and dropped on each of eight samples from the same patient in the same trigger group.

### Trigger Preparation

IFNα preparation
   1. Thawed IFN-α and PBS+0,1% BSA aliquots on ice. Once thawed, prepare IFN-α dilution (10-fold dilution) on ice
   2. Prepared the working solution by adding 180 μL of ice cold-PBS+0.1% BSA to the stock solution (20 μL of IFN-α per tube)
   3. Mixed by pipetting up and down 2-3 times
   4. Kept on ice and use it within 1 h after dilution
IL-2 preparation
   1. Thawed IL-2 and PBS+0,1% BSA aliquots on ice
   2. Prepared the working solution by adding 180 μL of ice cold-PBS+0.1% BSA to the stock solution (20 μL of IL-2 per tube)
   3. Mixed by pipetting up and down 2-3 times
   4. Kept on ice and use it within 1 h after dilution
IL-6 preparation
   1. Thawed one aliquot of PBS+0.1% BSA and one of IL-6 on ice
   2. Prepared the working solution by adding 180 μL of ice cold-PBS+0.1% BSA to the stock solution (20 μL of IL-6 per tube)
   3. Mix by pipetting up and down 2-3 times
   4. Kept on ice and use it within 1 h after dilution
IL-12 preparation
   1. Thawed IL-12 and PBS+0,1% BSA aliquots on ice
   2. Prepared the working solution by adding 180 μL of ice cold-PBS+0.1% BSA to the stock solution (20 μL of IL-12 per tube)
   3. Mixed by pipetting up and down 2-3 times
   4. Kept on ice and use it within 1 h after dilution
GM-CSF preparation
   1. Thawed GM-CSF and PBS+0,1% BSA aliquots on ice
   2. Prepared the working solution by adding 995 μL of ice cold-PBS+0.1% BSA to the stock solution (5 μL of GMCSF per tube)
   3. Mixed by pipetting up and down 2-3 times
   4. Kept on ice and use it within 1 h after dilution

### Materials

TABLE VI

| Name and producer | Cat. No. | Storage |
|---|---|---|
| Attune ™ focusing fluid, Thermo Fischer Scientific | 4449791 | RT |
| Attune ™ Wash Solution, Thermo Fischer Scientific | A24974 | RT |
| Attune ™ Shutdown Solution (1X), Thermo Fischer Scientific | A24975 | RT |
| Attune ™ Performance Tracking Beads | 4449754 | 4° C. |
| Rainbow Calibration Particles, 8 peaks, BioLegend | 422903 | 4° C. |
| Bovine Serum Albumin, Sigma | A3059-500G | 4° C. |
| DPBS, no calcium, no magnesium (1X), Gibco | 14190-250 | RT |
| V450 Mouse Anti-Human CD4 (Clone RPA-T4), BD Bioscience | 561838 | 4° C. |
| PE Mouse Anti-STATI (pY701) (Clone 4a), BD Bioscience | 562069 | 4° C. |
| Alexa Fluor ® 647 Mouse Anti-STAT3 (pY705) (Clone 4/P-STAT3), | 557815 | 4° C. |
| BD Bioscience | | |
| PE Mouse Anti-STAT5 (pY694) (Clone 47/Stat5(pY694)), | 612567 | 4° C. |
| BD Bioscience | | |
| Alexa Fluor ® 647 Mouse anti-Total STAT1 (Clone 1/STAT1), | 558560 | 4° C. |
| BD Bioscience | | |
| Alexa FluorR 488 Mouse Anti-STAT5 (Clone A9), Santa Cruz | sc-74442 | 4° C. |
| Biotechnology | | |
| PE Mouse Anti-Human CD335 (NKp46) (Clone 9E2/NKp46), | 557991 | |
| BD Bioscience | | |
| Alexa Fluor ® 647 Mouse Anti-Stat4 (pY693) (Clone 38/p-Stat4), | 558137 | |
| BD Bioscience | | |
| Falcon ® 5 mL Polystyrene Round Bottom, BD Falcon | 352054 | RT |
| Falcon ® 96-well Polystyrene Round Bottom plate, BD Falcon | 351177 | RT |

### 1.13. IFN-α In Vivo Challenge

Part 1: IFN-α-Induced Biomarkers (Neopterin, β-2-Microglobulin) were Measured Following In Vivo IFN-a-Alpha Challenge

### Blood Collection:

Following injection of IFN-α and administration of Compound 1, the neopterin and β2-microglobulin proteins were quantified in the serum collected 5 days after IFN-α challenge.
3 mL of blood was collected into serum tubes (3 mL Serum tube, clot activator, 13×75 mm, Vacuette Ref 454474 OR alternative: Ref 454095). In the event PK and PD samples are collected on the same timepoint as per the schedule of events table in the protocol, PD samples are always collected after the PK samples.

### Serum Preparation:

After collection in serum tubes, incubate in an upright position at room temperature for 30-45 min (no longer than 60 min) to allow clotting. If using a clot-activator tube, invert carefully 5-6 times to mix clot activator and blood before incubation.
Centrifuge for 15 min at manufacturer's recommended speed (usually 1000-2000 RCF) in a refrigerated centrifuge (4° C.). Do not use brake to stop centrifuge.
Inspect serum for turbidity. Turbid samples should be centrifuged and aspirated again to remove remaining insoluble matter.
Carefully aspirate the supernatant (serum) and transfer 500p of serum (500 μL per tube) into 2 individual clean polypropylene tubes (1.5 mL polypropylene microtubes (Clear, with natural colour screw cap, graduated, skirted, sterile) Cat No: 211-0099 (VWR))) the rest is divided in 2 additional tubes (backup). The samples should be maintained at 2-8° C. or on wet ice while handling. Store between −36° C. and −20° C. (keep away from direct sun light).

### Neopterin Measurement:

Neopterin ELISA provided by IBL International is an In Vitro Diagnostic (IVD) competitive ELISA, allowing quantifying Neopterin in human serum, plasma and urine.
In-study sample analysis was performed according to the technical manual of the manufacturer.
Each assay plate consisted of:
   5 non-zero Calibrators (CAL B-F: ready-to-use) provided with the kit,
   One zero Calibrator (CAL A: ready-to-use) provided with the kit,
   2 Quality controls (ready-to-use), provided with the kit,
   Up to 40 clinical samples tested not diluted (neat), according to Kit's Instructions.
All these standards, QCs and clinical samples were tested in duplicate.

### β2-Macroglobulin Measurement

VIDAS® β2 Microglobulin assay provided by Biomerieux is an In Vitro Diagnostic assay, allowing quantifying β2 microglobulin in human serum or plasma.
In-study sample analysis was performed according to the technical manual of the manufacturer.
The calibration was performed in duplicate, at the beginning of the study and was effective for 14 days. Similarly, 2 controls, a weak positive control (C1) and a strong positive control (C2) were tested within calibration step.

### Materials:

TABLE VII

| | MANUFACTURER | |
|---|---|---|
| SUPPLIER | REFERENCE | DESIGNATION |
| Biomérieux | 30420 | VIDAS β2-Microglobulin |
| IBL international | RE59321 | Neopterin ELISA |

Part 2: Gene Expression (mRNA Analysis) after In Vivo IFN-α Challenge

### Blood Collection:

Blood was collected by venipuncture directly in Paxgene Blood RNA Tubes following the manufacturer instructions and sent for RNAseq analysis to Genewiz Europe (Bahnhofstraße 86, 04158 Leipzig, Germany)

### Transcriptomic Analysis/RNA-Sequencing Data Processing and Analysis

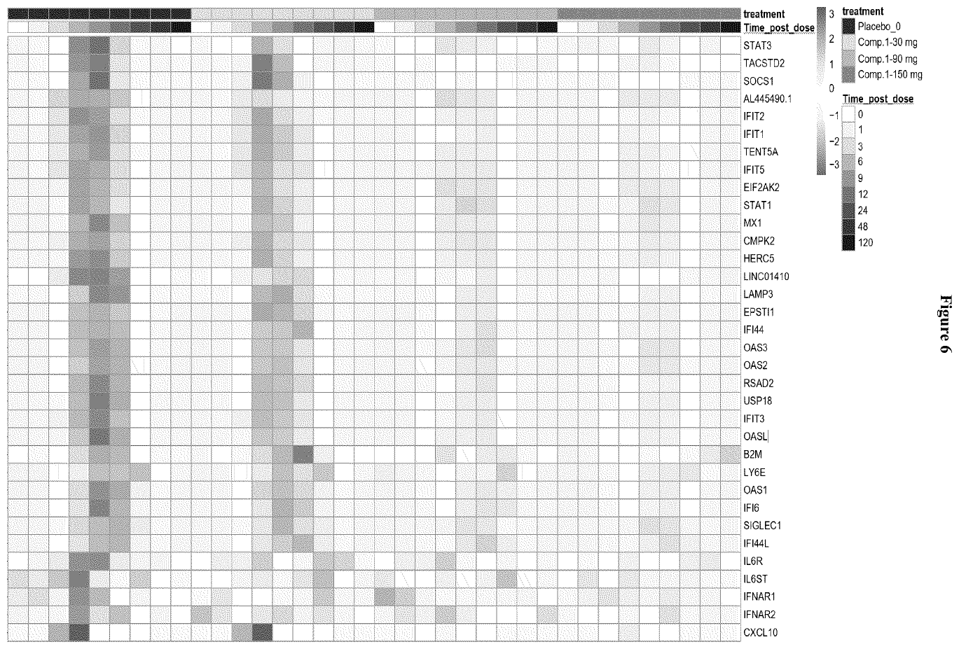

Whole blood total RNA of 3 cohorts of 8 subjects (2 placebo and 6 active treatment) was obtained using a standard extraction kit. RNA integrity number (RIN) values were evaluated and all the samples were analyzed. mRNA was obtained from total RNA using oligo (dT)beads, after globin depletion, and used to generate double-stranded cDNA fragments. The cDNA undergone paired-end repair to convert overhangs into blunt ends. After 3′-monoadenylation and adaptor ligation, cDNAs were purified. Next, cDNA was amplified by PCR using primers specific for the ligated adaptors. The generated libraries were submitted to quality control before being sequenced on an Illumina-HiSeq.
The gene expression levels were quantified using the pseudoalignment method of Kallisto version 0.46.0 with default parameters (Nicolas L Bray, Harold Pimentel, Páll Melsted and Lior Pachter, Near-optimal probabilistic RNA-seq quantification. Nature Biotechnology 34, 525-527 (2016), doi: 10.1038/nbt.3519).
The Ensembl human gene annotation version 100 (GRCh38) was chosen as reference for gene quantification. Differential expression analysis was performed with DESeq2 version 1.30.0 (Love M I, Huber W, Anders S. Moderated estimation of fold change and dispersion for RNA-seq data with DESeq2. Genome Biology, 15, 550 (2014), doi: 10.1186/s13059-014-0550-8).
In brief, for each gene included in DESeq2's model, a log 2 fold change (FC) was computed and a Wald test statistic was assessed with a P value and an adjusted P value. In this study, we consider a gene as differentially expressed (DE) when |log2FC|>1 and adjusted P value <0.05. For graphical visualization we selected genes that were differentially expressed in at least on condition. The heatmap shows the scaled expression values for each condition (mean normalized read counts, scaled to z-scores by gene). The R package ComplexHeatmap version 2.8.0 (Zuguang G, Roland Eils R, Schlesner M. Affiliations collapse Complex heatmaps reveal patterns and correlations in multidimensional genomic data, 15; 32(18):2847-9. Bioinformatics, (2016), doi: 10.1093/bioinformatics/btw313) was used to generate the heatmap represented in FIG. 6.

### 1.14. Conclusions

Administration of single ascending oral doses of 10 to 200 mg Compound 1 or placebo, and multiple ascending oral doses of 30 to 150 mg Compound 1 q.d. or placebo were considered safe and well tolerated in healthy male adults.

After single dosing of Compound 1 as an oral suspension in healthy male adults, a dose-proportional increase in exposure was observed in fasted state with no impact of food up to 90 mg. While absorption was saturated in fasted state at higher doses, higher exposures were achieved under fed conditions with linear increase in exposure up to 200 mg.

After repeated dosing of Compound 1 under fed conditions up to 150 mg q.d., there was no pronounced deviation from dose proportionality. Compound 1 was quickly absorbed with median tₘₐₓ ranging from 2 to 3.5 hours throughout dosing, and showed a bi-phasic decline with mean t_{1/2} ranging from 13.2 to 14.5 hours across doses.

Compound 1 showed a strong inhibitory effect on the IFN-α pathway after a single dose of Compound 1 and at steady state. A dose-response was observed leading to a complete inhibition of pSTAT1 during 24 hours post-dose at the highest dose levels used.

Dosing of Compound 1 at 150 mg QD completely inhibited IFNα-induced STAT1 and STAT3 phosphorylation but did not impact IL-2- and GM-CSF-induced STAT5 phosphorylation.

### Example 2. Drug-Drug Interaction Clinical Study

The study of the current example was an open-label, fixed-sequence, drug-drug interaction study to evaluate the effect of multiple oral doses of Compound 1 150 mg once daily (q.d.), a potential cytochrome P450 (CYP) 3A4 inhibitor, on the PK of a single dose of midazolam (MDZ), a sensitive index substrate of CYP3A4, in healthy subjects.

### 2.1. Study Endpoints

### Primary Outcome Measure was:

PK parameters of MDZ in plasma: area under the plasma concentration-time curve from time zero to infinity (AUC_{0-∞}) and maximum observed plasma concentration (Cₘₐₓ).
   Secondary Outcome Measures were:
PK parameters of Compound 1 in plasma: Cₘₐₓ, area under the plasma concentration-time curve over the dosing interval (AUCτ), and trough plasma concentration observed at the end of the dosing interval (Cτ).
Frequency and severity of treatment-emergent adverse events (TEAEs), treatment-emergent serious adverse events (SAEs), and TEAEs leading to treatment discontinuations.

### 2.2. Study Interventions

This was an open-label, fixed-sequence study to evaluate the effect of Compound 1 once daily (q.d.) on the PK of a single dose of MDZ, a sensitive index substrate of cytochrome P450 3A4, in healthy subjects.

### 2.3. Inclusion Criteria

To be eligible, the subjects met the following criteria:
Male or female between 18 and 55 years of age (extremes included), on the date of signing the informed consent form.
Female subjects should be of non-childbearing potential, defined as permanently surgically sterile (bilateral oophorectomy, i.e. surgical removal of ovaries, bilateral salpingectomy, i.e. surgical removal of the fallopian tubes, or hysterectomy, i.e. surgical removal of uterus), or with no menses for 12 or more months without an alternative medical cause AND a follicle-stimulating hormone level in the postmenopausal range. These female subjects must also have a negative pregnancy test. For surgical sterilization, documented confirmation will be requested.
A body mass index between 18.0 and 30.0 kg/m2, inclusive.
Judged to be in good health by the investigator based upon the results of a medical history, physical examination, vital signs, 12-lead electrocardiogram (ECG), and fasting clinical laboratory safety tests available at screening and prior to enrolment. Neutrophil, lymphocyte, and platelet counts must be above the lower limit of normal range. Total bilirubin, aspartate aminotransferase, and alanine aminotransferase must be within normal ranges. Other clinical laboratory safety test results must be within the reference ranges or test results that are outside the reference ranges need to be considered not clinically significant in the opinion of the investigator.

Key exclusion criteria: subjects meeting one or more of the following criteria were not selected for this study:
Known hypersensitivity to investigational product (IP) and/or MDZ ingredients or history of a significant allergic reaction to IP and/or MDZ ingredients as determined by the investigator.
Treatment with any medication (including over-the-counter and/or prescription medication, dietary supplements, nutraceuticals, vitamins and/or herbal supplements, and hormonal replacement therapy for postmenopausal subjects) except occasional paracetamol (maximum dose of 2 g/day and maximum of 10 g/2 weeks) in the last 2 weeks or 5 half-lives of the drug, whichever is longer, prior to the first dosing.

### 2.4. Study Design/Methodology

This was an open-label, fixed-sequence study to evaluate the effect of Compound 1 once daily (q.d.) on the PK of a single dose of MDZ, a sensitive index substrate of cytochrome P450 3A4, in healthy subjects.

A total of 14 subjects were enrolled and completed the study.

The screening period was from Day −21 until Day −2.

On Days 1 and 7, single dose of 2 mg MDZ as commercially available liquid formulation, containing 2 mg/mL MDZ administered orally under fed conditions.

From Day 3 to Day 8, 150 mg Compound 1 q.d. as 2 capsules of 75 mg administered orally under fed conditions.

A follow-up visit was conducted 14±3 days after the last study drug administration.

### 2.5. Pharmacokinetic Assessment

The effect of Compound 1 on the PK of MDZ was assessed on log-transformed (natural logarithm) MDZ parameters by means of a mixed-effect model with treatment day as fixed effect and subject as random effect. Point estimates of log-transformed PK parameters of MDZ in combination with Compound 1 as test treatment versus log-transformed PK parameters of MDZ alone as reference treatment were calculated as the geometric mean ratio (GMR), expressed as a percentage. To assess the potential interaction between Compound 1 and MDZ, the 90% confidence intervals (CIs) of the GMRs were calculated.

### 2.6. Pharmacokinetic Results

In vitro studies indicated that Compound 1 was a potential time-dependent inhibitor of CYP3A4. Therefore, the potential of Compound 1 to cause CYP3A4 mediated drug-drug interactions in vivo was evaluated using MDZ, a sensitive index substrate of CYP3A4. Compound 1 dosed at 150 mg q.d. resulted in a marginal increase in MDZ exposure of approximately 1.10-fold on Cₘₐₓ and 1.21-fold on AUC_{0-∞}. This indicates that Compound 1 did not cause clinically relevant inhibition of CYP3A4 and the time-dependent inhibition observed in vitro was not confirmed.

The arithmetic means (coefficient of variation [CV %]) and the GMRs (90% CI) of the main PK parameters of MDZ are provided in the table below.

TABLE VIII

| | | | Inferential Statistics |
|---|---|---|---|
| | Descriptive Statistics | | on MDZ PK Parameters |
| | on MDZ PK Parameters | | LS Geometric Mean Ratio |
| | Arithmetic Mean (CV %) | | (90% CI)^{a} |
| | | MDZ 2 mg + | MDZ 2 mg + |
| | | Compound 1 | Compound 1 |
| | MDZ 2 mg | 150 mg q.d. | 150 mg q.d. (Day 7) |
| | (Day 1) | (Day 7) | vs MDZ 2 mg (Day 1) |
| Parameters | (N = 14) | (N = 14) | (N = 14) |
| AUC_{0-∞} (ng · h/mL) | 23.5 (39.6) | 27.9 (41.4) | 121 (109; 133) |
| Cₘₐₓ (ng/mL) | 5.95 (37.8) | 6.24 (22.0) | 110 (95; 127) |

LS = least squares; N = number of subjects.
^{a}Point estimate and 90% CI of the least-squares GMR from a mixed-effects model on In-transformed values with treatment day as fixed effect and subject as random effect.

Overall, Compound 1 exposure at 150 mg q.d. was comparable when administered alone or co-administered with a single dose of MDZ 2 mg. Steady state seems to have been reached at the time of the evaluation of the drug-drug interaction, i.e. on Day 7.

### 2.7. Pharmacodynamic Results

2.7.1. Effect of Compound 1 on the IFN-α, IL6, and IL2 pSTAT Pathways

After multiple administrations of Compound 1 150 mg q.d. and ex vivo stimulation of whole blood cells with IFN-α, IL6, or IL2, pSTAT proteins were measured in CD4⁺ T-cells. The kinetics of the actual values (mean percentage positive cells) for the IFN-α, IL6, and IL2 pSTAT pathways over time were visually inspected on Day 6. When excluding potential incorrectly stimulated Day 1 predose values, an inhibition of the IFN-α/pSTAT1, IFN-α/pSTAT3, and IL6/pSTAT1 pathways by Compound 1 was observed on Day 6 predose, while for the IL6/pSTAT3 and IL2/pSTAT5 pathways Day 1 and Day 6 values were similar.

Visual inspection of PD data and PK/PD correlation plots, showed a concentration-dependent inhibition of the IFN-α/pSTAT1, IFN-α/pSTAT3, and IL6/pSTAT1 pathways by Compound 1 on Day 6. For the IL6/pSTAT3 and IL2/pSTAT5 JAK1-dependent pathways, no inhibition and no concentration-dependent effect was apparent, indicating a low sensitivity of these pathways to Compound 1.

### 27.2. STAT1/5 Expression in Unstimulated Condition

Actual values (percentage positive cells) for STAT1 and STAT5 in the unstimulated condition were visually inspected for changes over time. After 4 days of treatment with Compound 1, no apparent difference in the percentage of CD4⁺ T-cells expressing STAT1 or STAT5 was observed on Day 6.

### 2.8. Conclusions

Compound 1 dosed at 150 mg q.d. resulted in a marginal increase in MDZ exposure of approximately 1.10-fold on Cₘₐₓ and 1.21-fold on AUC_{0-∞}, indicating that Compound 1 did not cause clinically relevant inhibition of CYP3A4.

Compound 1 exposure at 150 mg q.d. was comparable when administered alone or coadministered with a single dose of MDZ 2 mg.

Multiple oral doses of Compound 1 150 mg q.d., administered with or without MDZ 2 mg, were well tolerated.

### Example 3. Drug-Drug Interaction Clinical Study

### 3.1. Title of Study

The study of the current example is a non-randomized, fixed-sequence, open-label, drug-drug interaction study to evaluate the effect of multiple doses of Itraconazole on the single dose pharmacokinetics of Compound 1 in adult, healthy subjects.

### 3.2. Study Objective

The primary objective of this study is to evaluate the effect of Itraconazole on the PK of Compound 1.

The secondary objective of this study is to evaluate the safety and tolerability of Compound 1 when administered alone or in combination with Itraconazole and to evaluate the PK of Itraconazole, to confirm relevant exposure for CYP3A4 and P-gp inhibition.

### 3.3. Study Endpoints

The primary outcome measure is Cₘₐₓ and AUC_{0-∞} of Compound 1.

The secondary outcome measures are:
Safety and tolerability of Compound 1 when administered alone or in combination with Itraconazole: safety and tolerability, assessed by the incidence and severity of TEAEs, treatment-emergent SAEs, and TEAEs leading to treatment discontinuation;
Effect of PK parameters of Itraconazole, to confirm relevant exposure for CYP3A4 and P-gp: C_{max ss}, AUC_{τ}, and C_{τ} of Itraconazole.

### 3.4. Study Design

This is an open-label, single fixed-sequence study comprising one cohort of 14 healthy male and female adult subjects to assess the impact of Itraconazole, a strong CYP3A4 inhibitor and strong P-gp inhibitor on the PK of Compound 1.

The study is divided in three different periods:
Screening period: Day −21 to Day −1
Open-label study period: Day 1 to Day 11, comprising the periods:
   i. Open-label study period 1: Day 1 to Day 4
   ii. Open-label study period 2: Day 5 to Day 11
Follow-up Period: 14 (±3) days after last administration of Compound 1 or Itraconazole

### 3.4.1. Study Design—Open-Label Study Period 1: Day 1 to Day 4

On Day 1 subjects receive a single oral dose of 25 mg of Compound 1, follow by a 72-hour PK blood sampling period.

### 3.4.2. Study Design—Open-Label Study Period 2: Day 5 to Day 11

Subjects receive oral q.d. doses of 200 mg Itraconazole as a solution (10 mg/mL).

On Day 8 subjects receive a single oral dose of 25 mg of Compound 1, co-administered with Itraconazole, follow by a 96-hour PK blood sampling period.

The study drugs are administered under fasting conditions on Day 1 (Compound 1 alone) and Day 8 (Compound 1+Itraconazole).

Subjects discharge from the clinical center on Day 11, on the condition that all required assessments are performed. Each subject is in the study for approximately 7 weeks (screening visit to follow-up visit). A follow-up visit is conducted 14±3 days after the last study drug administration.

### 3.4.3. Formulation Compound 1

Compound 1 is provided as a 25 mg capsule for oral administration.

### 3.4.4. Formulation Itraconazole

Itraconazole is administered as a 10 mg/mL oral solution (commercially available as Sponarox). Composition of the solution: each milliliter of Sponarox oral solution contains 10 mg of Itraconazole as well as hydroxypropyl-β-cyclodextrin, sorbitol, propylene glycol, hydrochloric acid, flavouring, sodium saccharin, sodium hydroxide and purified water.

### 3.4.5 Dosage Administration—Open-Label Study Period 1: Day 1 to Day 4

Subjects are instructed to not chew Compound 1 prior to swallowing, and to swallow the study drug as a whole.

On Day 1, subjects receive a single oral dose of 25 mg of Compound 1 together with 240 mL of water in the morning, after an overnight fast of at least 10 hours and remain fast up to 4 hours postdose.

On Day 1 to 4, fluid intake (including water) is prohibited from 1 hour prior to dose administration, except for water intake at dosing. Fluid intake is allowed ad libitum from 1 hour after dosing. No fluid restrictions apply on all other days. Subjects are encouraged to drink at least 1 liter of fluid per day throughout the study.

### 3.4.6. Dosage Administration—Open-Label Study Period 2: Day 5 to Day 11

Subjects is instructed to not chew Compound 1 prior to swallowing, and to swallow the study drug as a whole.

On Day 5 through Day 11 subjects receive doses of 200 mg q.d. of Itraconazole as an oral solution (10 mg/mL).

On Day 8, fluid intake (including water) is prohibited from 1 hour prior to dose administration. Then, a single oral dose of 25 mg Compound 1 is co-administered with Itraconazole in the morning, after an overnight fast of at least 10 hours and subjects remain fast up to 4 hours postdose.

On Days 5 to 7 and 9 to 10, a snack is served 30 minutes prior to Itraconazole dosing and breakfast is served 1 hour after Itraconazole intake.

All dose administrations are carried out by the investigator or by a member of the clinical center designated by the investigator.

On dosing days with Compound 1 (Days 1 and 8), fluid intake is prohibited from 1 hour prior to dose administration until 1 hour after dose administration, except for 240 mL water intake with dose administration. Fluid intake is allowed ad libitum from 1 hour after dosing. Subjects are advised to maintain a fluid intake of at least 1 liter per 24 hours. Fluid intake restrictions do not apply to other days.

### 3.5. Inclusion Criteria

To be eligible, the subjects must meet all of the following inclusion criteria:
Male or female subject between 18-55 years of age (extremes included), on the date of signing the ICF.
BMI between 18-30 kg/m², extremes included.
Judged to be in good health by the investigator based upon the results of a medical history, physical examination, vital signs, 12-lead electrocardiogram (ECG), and fasting clinical laboratory safety tests. Neutrophil, lymphocyte, and platelet counts must be above the lower limit of normal range. Total bilirubin, aspartate aminotransferase (AST), and alanine aminotransferase (ALT) must be within normal ranges. Other clinical laboratory safety test results must be within the normal ranges or test results that are outside the normal ranges need to be considered not clinically significant in the opinion of the investigator.
Subject must be able and willing to comply with restrictions on prior medication: all medication (including OTC and/or prescription medication, dietary supplements, nutraceuticals, vitamins and/or herbal supplements [e.g. St John's wort], oral hormonal contraceptives for WOCBP, and hormonal replacement therapy for postmenopausal subjects) except occasional paracetamol (maximum dose 2 g/day and a maximum of 10 g/2 weeks) should be discontinued at least 2 weeks or 5 half-lives of the drug, whichever is longer, prior to the first dose of Compound 1 administration and throughout the study.
Negative screen for drugs (amphetamines, barbiturates, benzodiazepines, cannabis, cocaine, opiates, methadone, tricyclic antidepressants) and alcohol.
Able and willing to comply with the CSP requirements and signing the ICF as approved by the IEC/IRB, prior to any screening evaluations.
Female subjects should be of non-childbearing potential, defined as permanently surgically sterile (bilateral oophorectomy, i.e. surgical removal of ovaries, bilateral salpingectomy or hysterectomy, i.e. surgical removal of uterus), or with no menses for 12 or more months without an alternative medical cause AND a follicle-stimulating hormone (FSH) level in the postmenopausal range. For surgical sterilization, documented confirmation will be requested.
   Female Subjects
   In line with the HMA's Clinical Trial Facilitation Group recommendation, female subjects are considered of non-childbearing potential if they meet one of the following criteria:
   No menses for 12 or more months without an alternative medical cause. A high FSH level in the postmenopausal range may be used to confirm a postmenopausal state in women not using hormonal contraception or hormonal replacement therapy. However in the absence of 12 months of amenorrhea, a single FSH measurement is insufficient.
   Permanently surgically sterile (bilateral oophorectomy, i.e. surgical removal of ovaries, bilateral salpingectomy or hysterectomy, i.e. surgical removal of uterus).
   All other female subjects are considered to be WOCBP and must use one of the following highly effective methods of birth control prior to the first dose of Compound 1, during the clinical study and for at least 35 days after the last dose of Compound 1:
      Intrauterine device (non-hormonal).
      Bilateral tubal occlusion.
      Sexual abstinence defined as refraining from heterosexual intercourse during the entire period of risk associated with the study treatments. The reliability of sexual abstinence needs to be evaluated in relation to the duration of the clinical study and the preferred and usual lifestyle of the subject.
   A female subject is not allowed to use any of the following contraception methods in this study:
      Combined (estrogen and progesterone containing) (oral, intravaginal, transdermal) hormonal contraception associated with inhibition of ovulation.
      Progesterone-only hormonal (oral, injectable, implantable) contraception associated with inhibition of ovulation.
      Intrauterine hormone-releasing system.
      Periodic abstinence (e.g. calendar, symptothermal, post-ovulation methods), declaration of abstinence for the duration of a clinical study, withdrawal, spermicides only, and lactational amenorrhea method.
   Hormonal contraceptives are not allowed due to the potential interference with the drug-drug interaction evaluation.
   In case a WOCBP has a vasectomized partner, provided that partner is the sole sexual partner of the WOCBP clinical study participant and that the vasectomized partner has received medical assessment of the surgical success, then she is not required to use an additional form of contraception.
   Within these limits, the specific forms of contraception employed are left to the discretion of the subject, the investigator, and/or the subject's physician.
   The safety of Compound 1 during breastfeeding is unknown. Nursing women are not allowed to take part in this clinical study.
   Male Subjects
   Non-vasectomized male subjects with female partners of childbearing potential must be willing to use a condom from the time of the first dose of IP, during the clinical study, and until FU, in addition to having their female partner use one of the following forms of contraception:
      Intrauterine device.
      Intrauterine hormone-releasing system.
      Combined (estrogen and progesterone containing) hormonal contraception associated with inhibition of ovulation (oral, intravaginal, transdermal).
      Progesterone-only hormonal contraception associated with inhibition of ovulation (oral, injectable, implantable).
   Sexual abstinence defined as refraining from heterosexual intercourse is considered a highly effective contraceptive measure only if it is the preferred and usual lifestyle of the subject. The reliability of sexual abstinence needs to be evaluated in relation to the duration of the clinical study.
   Periodic abstinence (e.g. calendar, symptothermal, post-ovulation methods), declaration of abstinence for the duration of a clinical study, withdrawal, spermicides only, and lactational amenorrhea method are not acceptable methods of contraception.
   In a case where the female partner of a male subject has undergone documented surgical sterilization that was performed more than 1 year before screening, the subject is not required to use an additional form of contraception.
   Vasectomized male subjects with female partners of childbearing potential are not required to use an additional form of contraception providing that surgical sterilization has been successful (documented azoospermia by semen analysis).
   No sperm donation is allowed from first dose of Compound 1 during the clinical study until FU.

### 3.6. Exclusion Criteria

Key exclusion criteria: subjects meeting one or more of the following criteria cannot be enrolled in this study:
History of serious allergic reaction to any drug as determined by the investigator (e.g. anaphylaxis requiring hospitalization) and/or known sensitivity to Compound 1 and/or Itraconazole or other antifungals or their excipients as determined by the investigator.
Positive serology for hepatitis B virus surface antigen (HBsAg) or hepatitis C virus (HCV), or history of hepatitis from any cause with the exception of hepatitis A that was resolved at least 3 months prior to first dosing.
Subject testing positive for SARS-CoV-2 infection as detected by real-time polymerase chain reaction (RT-PCR) or subjects who have been in contact with SARS CoV 2 infected individuals in the 2 weeks prior to first dosing. A subject presenting any signs or symptoms of SARS-CoV-2 infection, as detected at screening or baseline (e.g. cough, fever, headaches, fatigue, dyspnea, myalgia, anosmia, dysgeusia, anorexia, sore throat, etc.) (BMJ, 2020a; BMJ, 2020b), should undergo a rapid antigen test (and confirmatory RT-PCR test in case the antigen test is negative) and excluded if positive. Any other locally applicable standard diagnostic criteria may also apply to diagnose SARS-CoV-2 infection.
History of or a current immunosuppressive condition (e.g. HIV infection).
Having any illness, judged by the investigator as clinically significant for participation in the study, in the 3 months prior to dosing.
Presence or sequelae of gastrointestinal, liver, kidney (estimated glomerular filtration rate [eGFR]<90 mL/min/1.73 m², using the CKD-EPI formula) or other conditions known to interfere with the absorption, distribution, metabolism, or excretion of drugs.
History of malignancy within the past 5 years prior to screening, with the exception of excised and curatively treated non-metastatic basal cell carcinoma or squamous cell carcinoma of the skin or carcinoma in situ of cervix which is considered cured with minimal risk of recurrence.
History or presence of clinically significant abnormalities detected on 12-lead ECG of either rhythm or conduction e.g. known long QT syndrome or a QTcF>450 ms detected on the 12-lead ECG. A first-degree atrioventricular block will not be considered as a significant abnormality.
Male subjects not willing to comply with the contraceptive methods described in the Inclusion criteria, section 1.5.
Female subject is pregnant or breast feeding or intending to become pregnant or breastfeed during the study.
Significant blood loss (including blood donation [>450 mL]), or transfusion of any blood product within 12 weeks prior to screening.
Treatment with any drug known to have a potential for major organ toxicity in the last 3 months prior to dosing.
Treatment with any medication (including over-the-counter [OTC] and/or prescription medication, dietary supplements, nutraceuticals, vitamins and/or herbal supplements, and hormonal replacement therapy) except occasional paracetamol (maximum dose of 2 g/day and maximum of 10 g/2 weeks) in the last 2 weeks or 5 half-lives of the drug, whichever is longer, prior to the first dosing.
Active drug abuse or alcohol abuse (alcohol abuse defined as regular weekly intake of more than 14 units in which 1 unit=25 mL spirits, 125 mL wine, 250 mL beer or lager) within 2 years prior to dosing.
Active smoker and/or has used nicotine or nicotine-containing products within the past 6 months prior to dosing.
Regular consumption of a large quantity of caffeinated coffee, tea (>6 cups per day) or equivalent.
Concurrent participation or participation in a drug, drug/device, or biologic investigational research study within 12 weeks or 5 half-lives of the investigational product, whichever is longer, prior to first dosing.
Any condition or circumstances that in the opinion of the investigator may make a subject unlikely or unable to complete the study or comply with study procedures and requirements.

### 3.7. Pharmacokinetic Assessment

### 3.7.1. Blood Samples for Determination of Compound 1 in Plasma

Samples are collected by venipuncture (or indwelling cannula) preferably in the forearm into tubes containing K₂EDTA and are immediately chilled (ice bath). Within 30 min after blood collection, the plasma is separated in a refrigerated centrifuge at 4° C. for 10 min at circa 1500 g′ and transferred into tubes as described in the laboratory manual. The plasma samples are stored at <=−65° C. at the clinical center until shipment to the bioanalytical laboratory.

### 3.7.2. Blood Samples for Determination of Itraconazole in Plasma

Blood samples are collected by venipuncture (or indwelling cannula) preferably in the forearm into tubes containing K2EDTA and are immediately chilled (ice bath). Within 30 min after blood collection, the plasma is separated in a refrigerated centrifuge at 4° C. for 10 min at circa 1500 g′ and transferred into tubes as described in the lab manual. The plasma samples are stored at <=−20° C. at the clinical center until shipment to the bioanalytical laboratory.

The following parameters, where appropriate, are determined for Compound 1 and Itraconazole by non-compartmental analysis using individual concentration-time profiles in plasma:

For Compound 1 (Days 1 and 8):
Cₘₐₓ
tₘₐₓ
AUC₀₋ₜ
AUC_{0-∞}
t_{1/2,λ,z}
λz
CL/F
Vd/F

For Itraconazole (Day 8):
Cₜ
C_{max ss}
tₘₐₓ
AUC_{τ}

### 3.8. Pharmacokinetic Analysis and Assessment of Drug Interaction

All pharmacokinetic analyses are performed for subjects who have available and evaluable data (e.g. excluding all protocol deviations or AEs that may have an impact on the PK analysis).

Descriptive statistics are calculated by treatment for the plasma concentrations and the listed PK parameters. Mean ±SD plasma concentrations of Compound 1 and Itraconazole versus time are plotted per treatment.

Baseline is defined as the last available assessment prior to the first intake of Compound 1.

The effect of the Itraconazole on the PK of Compound 1 is assessed on log-transformed Compound 1 PK parameters Cₘₐₓ and AUC_{0-∞} by means of a mixed-effect model with treatment days as fixed effect and subject as random effect. Point estimate is calculated as the geometric mean of the individual ratios of each parameter for the test/reference treatments and expressed as a percentage. The 90% CI of the point estimates is calculated using the mean square error of the analysis of variance. Point estimate and 90% CI is calculated using Day 8 (Compound 1+Itraconazole) as test treatment versus Day 1 (Compound 1 alone) as reference treatment.

### 3.9. Study Results

The results of this clinical study can be found in Table VIIIa: compound 1 results in a AUC₀₋ₜ of 3120 and concomitant use of compound 1 with Itraconazole results in a AUC₀₋ₜ of 5750, which corresponds to a 1.8 fold increase in the AUC₀₋ₜ.

TABLE VIIIa
Clinical results:
Pharmacokinetic parameters compound 1

| | Day 1 - Compound 1 | | Day 8 - Compound 1 + | |
|---|---|---|---|---|
| | alone (n = 14) | | Itraconazole (n = 14) | |
| Cₘₐₓ (ng/mL) | 236 | (21.7)* | 267 | (38.6)* |
| tₘₐₓ (h) | 2 | (1-4)** | 3 | (1.5-5)** |
| AUC₀₋ₜ (ng · h/mL) | 3120 | (36.1)* | 5750 | (34.7)* |
| t_{1/2}(h) | 15.1 | (27.6)* | 23.4 | (20.3)* |

Comparison Day 8 vs Day 1: PE (90% CI) of GMR in %

| | |
|---|---|
| Cₘₐₓ | 112 (96; 131)*** |
| AUC₀₋ₜ | 188 (165; 214)*** |
| AUC_{0-∞} | 196 (173; 222)*** |

*, *, ***for number(s) between brackets → *= mean (CV %); **= median (range); ***= upper & lower intervals

### Example 4. Phase 1b Clinical Study

The study of the current example was a randomized, double-blind, placebo-controlled study to evaluate the safety, tolerability, and efficacy of Compound 1 in subjects with moderate to severe plaque psoriasis.

The primary objectives of this study were to evaluate the safety and tolerability of Compound 1 compared to placebo in subjects with moderate to severe plaque psoriasis and to evaluate signs of clinical efficacy of Compound 1 compared to placebo in subjects with moderate to severe plaque psoriasis.

### 4.1. Study Endpoints

### Primary Outcome Measure was:

Frequency and severity of treatment-emergent adverse events (TEAEs), treatment-emergent serious adverse events (SAEs), and TEAEs leading to treatment discontinuation in subjects with moderate to severe plaque psoriasis.
Psoriasis Area and Severity Index (PASI) % changefrom baseline at Week 4.
   Secondary Outcome Measures were:
Observed Compound 1 plasma trough concentrations(Ctrough).
Change from baseline in selected serum/plasma protein (e.g. interleukin 17 [IL-17]) levels between treatment groups and time points.
   Other Outcome Measures were:
Exploratory Efficacy
   PASI % change from baseline at Weeks 1, 2, 6 and 8.
   A reduction of at least 50%/75%/90%/100% in the PASI score relative to baseline at Weeks 1, 2, 4, 6 and 8.
   Change from baseline in affected body surface area (BSA) at Weeks 1, 2, 4, 6 and 8.
   Change from baseline in static Physician's Global Assessment of psoriasis (sPGA) (3 components+total) at Weeks 4 and 8.
   A Dermatology Quality of Life Index (DLQI) change from baseline at Weeks 4 and 8 (PRO questionnaire, 10 questions).
Exploratory Pharmacodynamics
   Change from baseline in RNA expression level between treatment groups and time points.
   Change from baseline in RNA expression and/or protein level in blood after ex vivo challenge.

### 4.2. Study Interventions

This was a randomized, double-blind, placebo-controlled study to evaluate the safety, tolerability, and efficacy of Compound 1 in subjects with moderate to severe plaque psoriasis.

### 4.3. Inclusion Criteria

To be eligible, the subjects met the following criteria:
Subjects must be male or female between 18-64 years of age (extremes included), on the date of signing the informed consent form (ICF).
Subject must be diagnosed (for at least 6 months before screening) of moderate to severe intensity plaque psoriasis. Subject's plaque psoriasis must be stable, defined asno flare during the month before the screening visit and no change of the severity between the screening visit and baseline visit.
At screening and at baseline (Day 1, predose), PASI >=12 (moderate to severe) andplaque-type psoriasis covering at least 10% of total body surface area (BSA).
At screening a Physician's Global Assessment (PGA) score of 3 (“moderate”) or 4(“severe”).
Subject must be considered by dermatologist investigator to be a candidate for systemic therapy of plaque psoriasis (either naïve or history of previous systemictreatment).

Key exclusion criteria: subjects meeting one or more of the following criteria were not selected for this study:
Subject has a known hypersensitivity to IP ingredients or history of a significantallergic reaction to IP ingredients as determined by the investigator.
Subjects with psoriasis other than plaque type or complicated psoriasis such as guttate, erythrodermic, exfoliative, inverse, pustular, palmo plantar, infected, or ulcerated psoriasis.
Subject has evidence of skin conditions other than psoriasis (e.g. eczema) at the time ofscreening or baseline visit that would interfere with the evaluation of psoriasis.
Subject is unable to discontinue prohibited therapies for the treatment of plaque psoriasis and/or cannot discontinue phototherapy (ultraviolet B [UVB] or psoralen andultraviolet A [PUVA]) before the start of the study up to the end of the study.
Subjects with current or a known or suspected history of immunosuppressive condition, history of invasive opportunistic infections (e.g. human immunodeficiency virus [HIV] infection, histoplasmosis, listeriosis, coccidioidomycosis, pneumocystosis, aspergillosis, or organ or bone marrow transplantation).
Subjects having an active clinically significant infection or any infection requiring oral or systemic therapy within 2 weeks prior screening or subjects currently on any chronicoral or systemic anti-infective therapy for chronic infection.
Subject testing positive for SARS-CoV-2 infection as detected at screening based on real time polymerase chain reaction (RT-PCR) or at baseline based on IgM immunoassay, or subjects who have been in contact with SARS-CoV-2 infected individuals in the two weeks prior to first dosing of IP. Subjects presenting any signs or symptoms of SARS-Cov-2 infection as detected at screening or baseline following careful physical examination (e.g. cough, fever, headaches, fatigue, dyspnea, myalgia, anosmia, dysgeusia, anorexia, sore throat, etc.). In addition, any other locally applicable standard diagnostic criteria may also apply to diagnose SARS-CoV-2 infection.
Subjects with evidence of active or latent infection with Mycobacterium tuberculosis(TB) as defined by:
   Positive QuantiFERON-TB Gold test result, AND/OR
   Chest radiograph (posterior anterior view) taken within 12 weeks prior to screening, read by a qualified radiologist or pulmonologist, with evidence of current active TB or old inactive TB.

Subjects with a history of TB who have successful treatment documentation areeligible for the study.

### 4.4. Study Design/Methodology

This was a Phase 1b randomized, double-blind, placebo-controlled multicenter study to evaluate the safety, tolerability, efficacy, PK, and PD of orally administered multiple q.d. doses of Compound 1 of 50 mg or 150 mg or of placebo for 28 days in subjects with moderate to severe plaque psoriasis.

31 subjects were randomized 11:10:10 to three treatment groups: 50 mg once daily (q.d.) Compound 1, 150 mg q.d. Compound 1, and placebo q.d..

Compound 1 and placebo were administered as capsules in a fed state.

Subjects were screened within a maximum of 28 days before the start of the treatment period. Subjects visited the clinical site on Day 1 (start of treatment), Day 8±1, Day 15±2, and Day 28±2. Follow-up visits were 14±2 and 28±2 days after the last investigational product (IP) intake.

### Safety Assessments

Safety assessments were performed throughout the study and included assessment of adverse events (AEs), clinical laboratory safety parameters, vital signs, physical examinations, and 12-lead electrocardiograms (ECGs).

### Efficacy Assessments

Efficacy was Assessed by the Investigator Using the PASI, the Extent of BSA Affected and the sPGA.

### Psoriasis Area and Severity Index (PASI)

The PASI combines assessments of the extent of body-surface involvement in 4 anatomical regions (head, trunk, arms, and legs) and the severity of desquamation, erythema, and plaque induration/infiltration (thickness) in each region, yielding an overall score of 0 for no psoriasis and up to a maximum score of 72 for severe disease. The PASI is the most commonly used and generally accepted primary endpoint and measure of psoriasis severity in clinical trials (EMEA, 2004).

### Body Surface Area Affected (BSA)

The percentage of BSA affected was evaluated on the visits. The percentage BSA affected is defined as the percent of BSA involvement, where 1% is approximately the area of the patient's handprint.
   Static Physician's Global Assessment (sPGA) Score
The sPGA score was evaluated on the visits. The sPGA of psoriasis is used to determine the subject's psoriasis lesions overall at a given time point. This scale does not have a recall period. The subject's psoriasis disease activity was assessed by a physician, using a 6-point scale, which ranges from 0 (cleared) to 5 (severe).

### Dermatology Quality of Life Index (DLQI)

The DLQI was evaluated on the visits. The DLQI is a simple, patient-administered, 10-question, validated, quality-of-lifequestionnaire that covers 6 domains including symptoms and feelings, daily activities, leisure, work and school, personal relationships, and treatment. Response categories include “not at all,” “a little,” “a lot,” and “very much,” with corresponding scores of 0, 1, 2, and 3 respectively and unanswered (“not relevant”) responses also scored as “0.” The recall period is 1 week

### 4.5. Pharmacokinetic Assessment

Blood samples for the Compound 1 PK assessment were collected on the visits. Samples were obtained by venipuncture (or indwelling cannula) into tubes containing K2EDTA and were immediately chilled (ice bath). Within 30 minutes after blood collection, the plasma was separated in a refrigerated centrifuge at 4° C. for 10 minutes at circa 1500 g and transferred into tubes. The plasma samples were stored at approximately −20° C. at the site until shipment to the central laboratory.

### 4.6. Pharmacodynamic Assessment

### Blood Cytokine PD Markers

Blood samples for protein analysis were collected by venipuncture (or indwelling cannula) into serum separating tubes (serum) and/or tubes containing K2EDTA (plasma), processed, and stored as specified in the laboratory manual. In addition, samples for the ex vivo challenge experiment (see Section ‘Ex vivo challenge experiment’ below) should be collected into tubes containing K2EDTA and/or TruCulture tubes, processed, and stored. Blood samples collected at predose on Day 1 and thereafter will be processed for serum and/or plasma. Processed samples will be shipped to the central laboratory and thereafter forwarded to the bioanalytical laboratory at which serum and/or plasma may be used to determine protein levels (e.g. IL-17), and other potential markers in response to daily IP administration.

### Ex Vivo Challenge Experiment

Blood samples for ex vivo challenge with cytokine (e.g. IFNα) were collected pre- and postdose at Day 1 and Day 15, processed and stored for shipment as specified in the laboratory manual. Processed samples were shipped to the central laboratory and thereafter forwarded to the bioanalytical laboratory at which samples were used to determine protein (e.g. IL-17), and/or other potential markers.

### Blood RNA PD Markers

Blood samples for RNA analysis were collected by venipuncture (or indwelling cannula) into PAXgene BloodRNA Tubes, processed, and stored. In addition, samples for the ex vivo challenge experiment were collected into tubes containing K2EDTA and/or TruCulture tubes, processed, and stored. Blood samples collected at predose on Day 1 and thereafter were processed and stored for shipment. Processed samples were shipped to the central laboratory and thereafter forwarded to the bioanalytical laboratory at which samples may be used to determine RNA expression in response to daily IP administration.

### Ex Vivo Challenge Experiment

Blood samples for ex vivo challenge with cytokine (e.g. IFNα) were collected pre- and post-dose, at Day 1 and Day 15, processed and stored for shipment. Processed samples were shipped to the central laboratory and thereafter forwarded to the bioanalytical laboratory at which samples were used to determine RNA expression.

The baseline disease characteristics for the patient population, including their distribution in the respective strata, is given in Table IX below.

TABLE IX

| | | | |
|---|---|---|---|
| Compound 1 | Compound 1 | | All |
| 50 mg | 150 mg | Placebo | Subjects |
| (N = 11) | (N = 10) | (N = 10) | (N = 31) |

Duration of psoriasis (years)

| | | | | |
|---|---|---|---|---|
| Mean (SD) | 16.3 (7.3) | 14.0 (7.3) | 11.7 (8.9) | 14.1 (7.8) |
| Min-Max | 6.1-26.0 | 1.8-31.1 | 0.9-24.9 | 0.9-31.1 |

PASI score

| | | | | |
|---|---|---|---|---|
| Mean (SD) | 24.2 (11.7) | 19.1 (5.9) | 20.4 (9.3) | 21.3 (9.3) |
| Min-Max | 12.6-43.7 | 13.0-33.6 | 13.3-37.0 | 12.6-43.7 |

BSA (%)

| | | | | |
|---|---|---|---|---|
| Mean (SD) | 38.5 (25.8) | 25.6 (10.8) | 28.7 (20.5) | 31.2 (20.4) |
| Min-Max | 11.0-80.0 | 11.0-52.0 | 12.0-70.0 | 11.0-80.0 |

sPGA score

| | | | | |
|---|---|---|---|---|
| Mean (SD) | 3.2 (0.4) | 3.2 (0.4) | 3.2 (0.4) | 3.2 (0.4) |
| Min-Max | 3.0-4.0 | 3.0-4.0 | 3.0-4.0 | 3.0-4.0 |

DLQI score

| | | | | |
|---|---|---|---|---|
| Mean (SD) | 10.0 (3.4) | 10.1 (3.5) | 8.1 (4.0) | 9.4 (3.6) |
| Min-Max | 4.0-15.0 | 5.0-17.0 | 2.0-16.0 | 2.0-17.0 |

The PASI percentage change from baseline (% CfB—LS Means from MMRM) are reported in Table X below.

TABLE X

| | | | | | |
|---|---|---|---|---|---|
| | | | | Compound 1 | Compound 1 |
| Visit, | Compound 1 | Compound 1 | | 50 mg - | 150 mg - |
| Statistic | 50 mg | 150 mg | Placebo | Placebo | Placebo |

Week 1

| | | | | | |
|---|---|---|---|---|---|
| LS Mean (SE) | −3.6 (3.72) | −23.6 (3.87) | −13.7 (3.84) | 10.1 (5.37) | −9.9 (5.43) |
| 90% CI | (−9.9; 2.7) | (−30.2; −17.0) | (−20.2; −7.2) | (0.9; 19.2) | (−19.2; −0.7) |
| p-value | | | | 0.071 | 0.079 |

Week 2

| | | | | | |
|---|---|---|---|---|---|
| LS Mean (SE) | −10.8 (5.50) | −34.3 (5.73) | −20.0 (5.68) | 9.3 (7.95) | −14.3 (8.03) |
| 90% CI | (−20.1; −1.4) | (−44.1; −24.6) | (−29.7; −10.3) | (−4.3; 22.8) | (−28.0; −0.6) |
| p-value | | | | 0.254 | 0.086 |

Week 4

| | | | | | |
|---|---|---|---|---|---|
| LS Mean (SE) | −21.1 (6.66) | −40.8 (6.93) | −25.8 (6.87) | 4.7 (9.62) | −15.0 (9.72) |
| 90% CI | (−32.5; −9.8) | (−52.6; −29.0) | (−37.5; −14.1) | (−11.7; 21.1) | (−31.5; 1.6) |
| p-value | | | | 0.630 | 0.135 |

Follow-up 1

| | | | | | |
|---|---|---|---|---|---|
| LS Mean (SE) | −22.6 (9.01) | −30.1 (9.35) | −30.2 (9.28) | 7.6 (13.00) | 0.1 (13.12) |
| 90% CI | (−38.0; −7.3) | (−46.1; −14.2) | (−46.0; −14.4) | (−14.6; 29.7) | (−22.3; 22.4) |
| p-value | | | | 0.564 | 0.996 |

Follow-up 2

| | | | | | |
|---|---|---|---|---|---|
| LS Mean (SE) | −27.9 (9.44) | −29.8 (9.83) | −30.2 (9.75) | 2.3 (13.65) | 0.4 (13.79) |
| 90% CI | (−44.0; −11.8) | (−46.6; −13.1) | (−46.8; −13.6) | (−21.0; 25.5) | (−23.1; 23.9) |
| p-value | | | | 0.868 | 0.978 |

The PASI 50 Response Rate (90% CI) is reported in Table XI below:

TABLE XI

| Visit, | | | | Compound 1 | Compound 1 |
|---|---|---|---|---|---|
| Response Rate | Compound 1 | Compound 1 | | 50 mg - | 150 mg - |
| (90% CI) | 50 mg | 150 mg | Placebo | Placebo | Placebo |
| Week 1 | 0.0% | 10.0% | 0.0% | 0.0% | 10.0% |
| | (0.0%; 23.8%) | (0.5%; 39.4%) | (0.0%; 25.9%) | (−21.3%; 19.7%) | (−12.7%; 34.8%) |
| Week 2 | 0.0% | 20.0% | 0.0% | 0.0% | 20.0% |
| | (0.0%; 23.8%) | (3.7%; 50.7%) | (0.0%; 25.9%) | (−21.3%; 19.7%) | (−5.0%; 45.9%) |
| Week 4 | 0.0% | 40.0% | 10.0% | −10.0% | 30.0% |
| | (0.0%; 23.8%) | (15.0%; 69.6%) | (0.5%; 39.4%) | (−34.8%; 11.2%) | (−2.2%; 56.0%) |
| Follow-up 1 | 0.0% | 30.0% | 0.0% | 0.0% | 30.0% |
| | (0.0%; 23.8%) | (8.7%; 60.7%) | (0.0%; 25.9%) | (−21.3%; 19.7%) | (2.6%; 55.8%) |
| Follow-up 2 | 18.2% | 30.0% | 20.0% | −1.8% | 10.0% |
| | (3.3%; 47.0%) | (8.7%; 60.7%) | (3.7%; 50.7%) | (−30.4%; 26.0%) | (−21.2%; 39.0%) |

The PASI 75 Response Rate (90% CI) is reported in Table XII below:

TABLE XII

| Visit, | | | | Compound 1 | Compound 1 |
|---|---|---|---|---|---|
| Response Rate | Compound 1 | Compound 1 | | 50 mg - | 150 mg - |
| (90% CI) | 50 mg | 150 mg | Placebo | Placebo | Placebo |
| Week 1 | 0.0% | 0.0% | 0.0% | 0.0% | 0.0% |
| | (0.0%; 23.8%) | (0.0%; 25.9%) | (0.0%; 25.9%) | (−21.3%; 19.7%) | (−21.3%; 21.3%) |
| Week 2 | 0.0% | 0.0% | 0.0% | 0.0% | 0.0% |
| | (0.0%; 23.8%) | (0.0%; 25.9%) | (0.0%; 25.9%) | (−21.3%; 19.7%) | (−21.3%; 21.3%) |
| Week 4 | 0.0% | 10.0% | 0.0% | 0.0% | 10.0% |
| | (0.0%; 23.8%) | (0.5%; 39.4%) | (0.0%; 25.9%) | (−21.3%; 19.7%) | (−12.7%; 34.8%) |
| Follow-up 1 | 0.0% | 10.0% | 0.0% | 0.0% | 10.0% |
| | (0.0%; 23.8%) | (0.5%; 39.4%) | (0.0%; 25.9%) | (−21.3%; 19.7%) | (−12.7%; 34.8%) |
| Follow-up 2 | 0.0% | 20.0% | 0.0% | 0.0% | 20.0% |
| | (0.0%; 23.8%) | (3.7%; 50.7%) | (0.0%; 25.9%) | (−21.3%; 19.7%) | (−5.0%; 45.9%) |

The BSA percentage change from baseline (% CfB—LS Means from MMRM) are reported in Table XIII below.

TABLE XIII

| | | | | | |
|---|---|---|---|---|---|
| | | | | Compound 1 | Compound 1 |
| Visit, | Compound 1 | Compound 1 | | 50 mg - | 150 mg - |
| Statistic | 50 mg | 150 mg | Placebo | Placebo | Placebo |

Week 1

| | | | | | |
|---|---|---|---|---|---|
| LS Mean (SE) | 0.3 (2.56) | −3.6 (2.66) | −0.7 (2.63) | 1.0 (3.70) | −2.9 (3.72) |
| 90% CI | (−4.1; 4.7) | (−8.1; 0.9) | (−5.2; 3.8) | (−5.3; 7.3) | (−9.2; 3.4) |
| p-value | | | | 0.791 | 0.441 |

Week 2

| | | | | | |
|---|---|---|---|---|---|
| LS Mean (SE) | −6.8 (5.78) | −10.8 (5.99) | −4.6 (5.93) | −2.1 (8.35) | −6.2 (8.38) |
| 90% CI | (−16.6; 3.1) | (−21.0; −0.6) | (−14.7; 5.5) | (−16.3; 12.1) | (−20.4; 8.1) |
| p-value | | | | 0.801 | 0.468 |

Week 4

| | | | | | |
|---|---|---|---|---|---|
| LS Mean (SE) | −10.3 (8.24) | −14.5 (8.54) | −8.4 (8.45) | −1.9 (11.90) | −6.1 (11.94) |
| 90% CI | (−24.3; 3.8) | (−29.0; 0.1) | (−22.8; 6.0) | (−22.2; 18.4) | (−26.4; 14.2) |
| p-value | | | | 0.875 | 0.614 |

Follow-up 1

| | | | | | |
|---|---|---|---|---|---|
| LS Mean (SE) | −6.7 (14.89) | −1.3 (15.43) | −9.5 (15.26) | 2.8 (21.50) | 8.2 (21.57) |
| 90% CI | (−32.1; 18.6) | (−27.6; 24.9) | (−35.5; 16.5) | (−33.8; 39.4) | (−28.6; 44.9) |
| p-value | | | | 0.898 | 0.708 |

Follow-up 2

| | | | | | |
|---|---|---|---|---|---|
| LS Mean (SE) | −8.2 (15.27) | −4.3 (15.83) | −9.3 (15.66) | 1.1 (22.05) | 5.0 (22.13) |
| 90% CI | (−34.2; 17.8) | (−31.3; 22.7) | (−36.0; 17.4) | (−36.4; 38.7) | (−32.7; 42.7) |
| p-value | | | | 0.960 | 0.823 |

The sPGA percentage change from baseline (00 CfB—LS Means from MMRM) are reported in Table XIV below.

TABLE XIV

| | | | | | |
|---|---|---|---|---|---|
| | | | | Compound 1 | Compound 1 |
| Visit, | Compound 1 | Compound 1 | | 50 mg - | 150 mg - |
| Statistic | 50 mg | 150 mg | Placebo | Placebo | Placebo |

Week 4

| | | | | | |
|---|---|---|---|---|---|
| LS Mean (SE) | −0.3 (0.18) | −0.7 (0.19) | −0.5 (0.19) | 0.2 (0.26) | −0.2 (0.27) |
| 90% CI | (−0.6; 0.0) | (−1.0; −0.4) | (−0.8; −0.2) | (−0.2; 0.7) | (−0.7; 0.3) |
| p-value | | | | 0.414 | 0.467 |

Follow-up 2

| | | | | | |
|---|---|---|---|---|---|
| LS Mean (SE) | −0.5 (0.19) | −0.9 (0.20) | −0.4 (0.20) | −0.1 (0.28) | −0.5 (0.28) |
| 90% CI | (−0.8; −0.1) | (−1.2; −0.6) | (−0.7; −0.1) | (−0.5; 0.4) | (−1.0; −0.0) |
| p-value | | | | 0.818 | 0.088 |

The DLQI percentage change from baseline (% CfB—LS Means from MMRM) is reported in Table XV below.

TABLE XV

| | | | | | |
|---|---|---|---|---|---|
| | | | | Compound 1 | Compound 1 |
| Visit, | Compound 1 | Compound 1 | | 50 mg - | 150 mg - |
| Statistic | 50 mg | 150 mg | Placebo | Placebo | Placebo |

Week 4

| | | | | | |
|---|---|---|---|---|---|
| LS Mean (SE) | −2.7 (1.29) | −3.4 (1.35) | −3.8 (1.37) | 1.1 (1.90) | 0.4 (1.95) |
| 90% CI | (−4.9; −0.5) | (−5.7; −1.1) | (−6.2; −1.5) | (−2.1; 4.4) | (−2.9; 3.7) |
| p-value | | | | 0.560 | 0.848 |

Follow-up 2

| | | | | | |
|---|---|---|---|---|---|
| LS Mean (SE) | −2.7 (1.56) | −2.5 (1.64) | −2.9 (1.66) | 0.2 (2.30) | 0.4 (2.36) |
| 90% CI | (−5.4; −0.1) | (−5.3; 0.3) | (−5.8; −0.1) | (−3.7; 4.1) | (−3.6; 4.5) |
| p-value | | | | 0.938 | 0.852 |

Compound 1 was well tolerated in subjects with moderate to severe psoriasis after 4 weeks of treatment. A summary of efficacy at week 4 is stated below:
PASI 50 response rate: 40% on 150 mg vs 10% on placebo.
PASI 75 response rate: 10% on 150 mg vs 0% on placebo.
PASI mean 00 CfB: −42% on 150 mg vs −26% on placebo.

### FINAL REMARKS

It will be appreciated by those skilled in the art that the foregoing descriptions are exemplary and explanatory in nature, and intended to illustrate the invention and its preferred embodiments. Through routine experimentation, an artisan will recognize apparent modifications and variations that may be made without departing from the spirit of the invention. All such modifications coming within the scope of the appended claims are intended to be included therein. Thus, the invention is intended to be defined not by the above description, but by the following claims and their equivalents.

All publications, including but not limited to patents and patent applications, cited in this specification are herein incorporated by reference as if each individual publication are specifically and individually indicated to be incorporated by reference herein as though fully set forth.

It should be understood that factors such as the differential cell penetration capacity of the compound can contribute to discrepancies between the activity of the compound in the in vitro biochemical and cellular assays.

At least some of the chemical names of compound of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

### REFERENCES

Babon, J. J., Lucet, I. S., Murphy, J. M., Nicola, N. A., Varghese, L. N., 2014. The molecular regulation of Janus kinase (JAK) activation. Biochem. J. 462, 1-13. https://doi.org/10.1042/BJ20140712
Broekman, F., Giovannetti, E., Peters, G. J., 2011. Tyrosine kinase inhibitors: Multi-targeted or single-targeted? World J. Clin. Oncol. 2, 80-93. https://doi.org/10.5306/wjco.v2.i2.80
Danielson, P. B. 2002. “The Cytochrome P450 Superfamily: Biochemistry, Evolution and Drug Metabolism in Humans.” Current Drug Metabolism 3 (6): 561-97. https://doi.org/10.2174/1389200023337054.
Dendrou, C. A., Cortes, A., Shipman, L., Evans, H. G., Attfield, K. E., Jostins, L., Barber, T., Kaur, G., Kuttikkatte, S. B., Leach, O. A., Desel, C., Faergeman, S. L., Cheeseman, J., Neville, M. J., Sawcer, S., Compston, A., Johnson, A. R., Everett, C., Bell, J. I., Karpe, F., Ultsch, M., Eigenbrot, C., McVean, G., Fugger, L., 2016. Resolving TYK2 locus genotype-to-phenotype differences in autoimmunity. Sci. Transl. Med. 8, 363ra149. https://doi.org/10.1126/scitranslmed.aag1974
“Drug Development and Drug Interactions: Table of Substrates, Inhibitors and Inducers.” 2021. FDA. FDA. Jan. 21, 2021. https://www.fda.gov/drugs/drug-interactions-labeling/drug-development-and-drug-interactions-table-substrates-inhibitors-and-inducers.
Fabian, M. A., Biggs, W. H., Treiber, D. K., Atteridge, C. E., Azimioara, M. D., Benedetti, M. G., Carter, T. A., Ciceri, P., Edeen, P. T., Floyd, M., Ford, J. M., Galvin, M., Gerlach, J. L., Grotzfeld, R. M., Herrgard, S., Insko, D. E., Insko, M. A., Lai, A. G., Lélias, J.-M., Mehta, S. A., Milanov, Z. V., Velasco, A. M., Wodicka, L. M., Patel, H. K., Zarrinkar, P. P., Lockhart, D. J., 2005. A small molecule-kinase interaction map for clinical kinase inhibitors. Nat. Biotechnol. 23, 329-336. https://doi.org/10.1038/nbt1068
Gillooly, K., Zhang, Y., Yang, X., Zupa-Fernandez, A., Cheng, L., Strnad, J., Cunningham, M., Heimrich, E., Zhou, X., Chen, J., Chaudhry, C., Li, S., McIntyre, K., Carman, J., Moslin, R., Wrobleski, S., Weinstein, D., Burke, J., 2016. BMS-986165 Is a Highly Potent and Selective Allosteric Inhibitor of Tyk2, Blocks IL-12, IL-23 and Type I Interferon Signaling and Provides for Robust Efficacy in Preclinical Models of Systemic Lupus Erythematosus and Inflammatory Bowel Disease. ACR Meet. Abstr.
“Greene's Protective Groups in Organic Synthesis, 4th Edition|Wiley.” 2006. Wiley.Com. 2006. https://www.wiley.com/en-gb/Greene %27s+Protective+Groups+in+Organic+Synthesis %2C+4th+Edition-p-9780470053485.
Hu, Bingjie, Xin Zhou, Michael A. Mohutsky, and Prashant V. Desai. 2020. “Structure-Property Relationships and Machine Learning Mo.” Molecular Pharmaceutics 17 (9): 3600-3608. https://doi.org/10.1021/acs.molpharmaceut.0c00637.
Ince, Ibrahim, Catherijne A. J. Knibbe, Meindert Danhof, and Saskia N. de Wildt. 2013. “Developmental Changes in the Expression and Function of Cytoc.” Clinical Pharmacokinetics 52 (5): 333-45. https://doi.org/10.1007/s40262-013-0041-1.
König, Jörg, Fabian Müller, and Martin F. Fromm. 2013. “Transporters and Drug-Drug Interactions: Important Determinants.” Pharmacological Reviews 65 (3): 944-66. https://doi.org/10.1124/pr.113.007518.
Liu, Lichuan, Akintunde Bello, Mark J. Dresser, Donald Heald, Steven Ferenc Komiathy, Edward O'Mara, Mark Rogge, S. Aubrey Stoch, and Sarah M. Robertson. 2016. “Best Practices for the Use of Itraconazole as a Repla.” Journal of Clinical Pharmacology 56 (2): 143-51. https://doi.org/10.1002/jcph.562.
Lynch, Tom, and Amy Price. 2007. “The Effect of Cytochrome P450 Metabolism on Drug Resp.” American Family Physician 76 (3): 391-96.
Neubauer, H., Cumano, A., Müller, M., Wu, H., Huffstadt, U., Pfeffer, K., 1998. Jak2 Deficiency Defines an EssentialDevelopmental Checkpoint in DefinitiveHematopoiesis. Cell 93, 397-409. https://doi.org/10.1016/S0092-8674(00)81168-X
O'Shea, J. J., Plenge, R., 2012. JAKs and STATs in Immunoregulation and Immune-Mediated Disease. Immunity 36, 542-550. https://doi.org/10.1016/j.immuni.2012.03.014
Parganas, E., Wang, D., Stravopodis, D., Topham, D. J., Marine, J.-C., Teglund, S., Vanin, E. F., Bodner, S., Colamonici, OR., van Deursen, J. M., Grosveld, G., Ihle, J. N., 1998. Jak2 Is Essential for Signaling through a Variety of Cytokine Receptors. Cell 93, 385-395. https://doi.org/10.1016/S0092-8674(00)81167-8
Schwartz, D. M., Bonelli, M., Gadina, M., O'Shea, J. J., 2016. Type I/II cytokines, JAKs, and new strategies for treating autoimmune diseases. Nat. Rev. Rheumatol. 12, 25-36. https://doi.org/10.1038/nrrheum.2015.167
Sohn, S. J., Barrett, K., Abbema, A. V., Chang, C., Kohli, P. B., Kanda, H., Smith, J., Lai, Y., Zhou, A., Zhang, B., Yang, W., Williams, K., Macleod, C., Hurley, C. A., Kulagowski, J. J., Lewin-Koh, N., Dengler, H. S., Johnson, A. R., Ghilardi, N., Zak, M., Liang, J., Blair, W. S., Magnuson, S., Wu, L. C., 2013. A Restricted Role for TYK2 Catalytic Activity in Human Cytokine Responses Revealed by Novel TYK2-Selective Inhibitors. J. Immunol. 191, 2205-2216. https://doi.org/10.4049/jimmunol.1202859
Teo, Yi Ling, Han Kiat Ho, and Alexandre Chan. 2015. “Metabolism-Related Pharmacokinetic Drug-drug Inter.” British Journal of Clinical Pharmacology 79 (2): 241-53. https://doi.org/10.1111/bcp.12496.
Vainchenker, W., Dusa, A., Constantinescu, S. N., 2008. JAKs in pathology: Role of Janus kinases in hematopoietic malignancies and immunodeficiencies. Semin. Cell Dev. Biol. 19, 385-393. https://doi.org/10.1016/j.semcdb.2008.07.002
Wandel, C., R. B. Kim, S. Kajiji, P. Guengerich, G. R. Wilkinson, and A. J. Wood. 1999. “P-Glycoprotein and Cytochrome P-450 3A Inhibition: Dissociation Of” Cancer Research 59 (16): 3944-48.

1. Compound 1 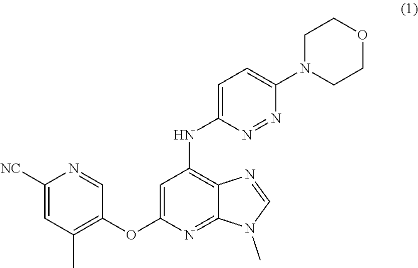 [SMILES: Cc1cc(C#N)ncc1Oc1cc(Nc2ccc(N3CCOCC3)nn2)c2ncn(C)c2n1]
or a pharmaceutically acceptable salt/cocrystal thereof, or a solvate or the solvate of a salt/cocrystal thereof, for use in the treatment of an inflammatory disease, a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, ulcerative colitis and/or Crohn's disease, wherein compound 1 is administered at a total daily dosage of at least 80 mg per day to 200 mg per day.

2. The use of compound 1, or a pharmaceutically acceptable salt/cocrystal thereof, or a solvate or the solvate of a salt/cocrystal thereof, in the manufacture of a medicament for the treatment of an inflammatory disease, a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, ulcerative colitis and/or Crohn's disease, wherein compound 1 is administered at a total daily dosage of at least 80 mg per day to 200 mg per day.

3. A method of treating an inflammatory disease, a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, ulcerative colitis and/or Crohn's disease, comprising administering to a patient compound 1, or a pharmaceutically acceptable salt/cocrystal thereof, or a solvate or the solvate of a salt/cocrystal thereof, wherein compound 1 is administered at a total daily dosage of at least 80 mg per day to 200 mg per day.

4. Compound 1 for use, use of compound 1 or method according to any of claims 1, 2 or 3, wherein the total daily dosage of compound 1 is administered as a once per day dosage (q.d.).

5. Compound 1 for use, use of compound 1 or method according to any of claims 1, 2, 3 or 4, wherein compound 1 is administered at a total daily dosage of at least 90 mg per day to 200 mg per day, preferably 150 mg per day to 200 mg per day.

6. Compound 1 for use, use of compound 1 or method according to any of claims 1, 2, 3, 4 or 5, wherein compound 1 is administered at a total daily dosage of 90 mg per day, 100 mg per day, 110 mg per day, 120 mg per day, 125 mg per day, 130 mg per day, 140 mg per day, 150 mg per day, 160 mg per day, 170 mg per day, 175 mg per day, 180 mg per day, 190 mg per day or 200 mg per day.

7. Compound 1 for use, use of compound 1 or method according to any of claims 1, 2, 3, 4, 5 or 6, wherein compound 1 is administered at a total daily dosage of 90 mg per day or 150 mg per day or 200 mg per day.

8. Compound 1 for use, use of compound 1 or method according to any of claims 1, 2, 3, 4, 5, 6 or 7, wherein compound 1 is administered orally.

9. Compound 1 for use, use of compound 1 or method according to any of claims 1, 2, 3, 4, 5, 6, 7 or 8, wherein compound 1 is administered orally to a patient in a fed state.

10. Compound 1 for use, use of compound 1 or method according to any of claims 1, 2, 3, 4, 5, 6, 7, 8 or 9, wherein the treatment or treating further comprises avoiding or contraindicating or discontinuing concomitant use or co-administration one or more compounds that are CYP inhibitors and/or P-gp inhibitors.

11. Compound 1 for use, use of compound 1 or method according to any of claims 1, 2, 3, 4, 5, 6, 7, 8, 9 or 10, wherein the patient in need of therapy thereof is currently undergoing treatment with one or more compounds that are CYP inhibitors and/or P-gp inhibitors.

12. Compound 1 for use, use of compound 1 or method according to any of claims 1, 2, 3, 4, 5, 6, 7, 8, 9, 10 or 11, wherein the use or method comprises the step of discontinuing the use of or treatment of one or more compounds that are CYP inhibitors and/or P-gp inhibitors prior to or at the same time as the step of starting the therapy.

13. Compound 1 for use, use of compound 1 or method according to any of claims 1, 2, 3, 4, 5, 6, 7, 8, 9, 10, 11 or 12, wherein the use or method comprises the step of discontinuing the use of or treatment of one or more compounds that are CYP inhibitors and/or P-gp inhibitors, at least 12 hours, preferably at least 24 hours, prior to commencing therapy thereof.

14. Compound 1 for use, use of compound 1 or method according to any of claims 1, 2, 3, 4, 5, 6, 7, 8, 9, 10, 11, 12 or 13, wherein the use or method further comprises concomitant use or co-administration of a medicament from the same class or mechanism of action or known to be a suitable alternative medicament for the respective therapy thereof and that is a medicament or one or more compounds that are not CYP inhibitors and/or P-gp inhibitors, particularly not CYP3A4 inhibitors and/or not P-gp inhibitors, and more particularly not strong CYP3A4 inhibitors and/or strong not P-gp inhibitors.

15. Compound 1 for use, use of compound 1 or method according to any of claims 1, 2, 3, 4, 5, 6, 7, 8, 9, 10, 11, 12, 13 or 14, wherein the use or method comprises discontinuing treatment with one or more compounds that are CYP inhibitors and/or P-gp inhibitors and commencing treatment with a medicament from the same class or mechanism of action or known to be a suitable alternative medicament for the respective therapy thereof and that is a medicament or one or more compounds that are not CYP inhibitors and/or P-gp inhibitors, particularly not CYP3A4 inhibitors and/or not P-gp inhibitors, and more particularly not strong CYP3A4 inhibitors and/or strong not P-gp inhibitors.

16. A pharmaceutical composition comprising a pharmaceutically acceptable carrier and a pharmaceutically effective amount of compound 1 for use, use of compound 1 or method according to any of claims 1, 2, 3, 4, 5, 6, 7, 8, 9, 10, 11, 12, 13, 14 or 15.

17. A package or kit comprising:
i. the compound according to Formula I or a pharmaceutically acceptable salt/cocrystal thereof, or a solvate or the solvate of a salt/cocrystal thereof, and
ii. a package insert, package label, instructions or other labelling comprising instructions to avoid or discontinue or contraindication of concomitant use or co-administration of one or more compounds that are CYP inhibitors and/or P-gp inhibitors.

18. A package or kit according to claim 17 further comprising one or more of the features according to any of claims 1, 2, 3, 4, 5, 6, 7, 8, 9, 10, 11, 12, 13, 14, 15 or 16.

19. Compound 1 for use, use of compound 1 or method according to any of claims 1, 2, 3, 4, 5, 6, 7, 8, 9, 10, 11, 12, 13, 14 or 15, or the kit according to claim 17 or 18, wherein the CYP inhibitor is a CYP3A4 inhibitor.

20. Compound 1 for use, use of compound 1 or method according to any of claims 1, 2, 3, 4, 5, 6, 7, 8, 9, 10, 11, 12, 13, 14, 15 or 19, or the kit according to claim 17, 18 or 19, wherein the CYP inhibitor is a CYP3A4 inhibitor and is one or more medicaments selected from Atazanavir, Boceprevir, Clarithromycin, Cobicistat, Conivaptan, Danoprevir, Darunavir, Delavirdine, Diltiazem, Elvitegravir, grapefruit juice, Idelalisib, Indinavir, Itraconazole, Ketoconazole, Lonafarnib, Lopinavir, Nefazodone, Nelfinavir, Nilotinib, Posaconazole, Ritonavir, Saquinavir, Stiripentol, Telithromycin, Tipranavir, Troleandomycin, Voriconazole, Aprepitant, Ciprofloxacin, Crizotinib, Cyclosporine, Dronedarone, Erythromycin, Fluconazole, Fluvoxamine, Imatinib, Verapamil, Chlorzoxazone, Cilostazol, Cimetidine, Fosaprepitant, Istradefylline, Ivacaftor, Lomitapide, Ranitidine, Ranolazine, Ticagrelor, (S)-omeprazole (esomeprazole)-high dose, ACT-178882, ACT-539313, Almorexant, AMD070, ANS-6637, Apararenone, ASP8477, Atorvastatin, AZD2327, Azithromycin, Berberine, Berotralstat, Bicalutamide, Brodalumab, Casopitant, Ceritinib, Clotrimazole, cranberry juice, Duvelisib, Entrectinib, Evacetrapid, Everolimus, Faldaprevir, Fedratinib, Fenebrutinib, FK1706, Fostamatinib, ginkgo (Ginkgo biloba), Glecaprevir/Pibrentasvir, Goldenseal (Hydrastis canadensis), Grazoprevir (ingredient of Zepatier), GSK2248761, Isavuconazole, Lapatinib, Larotrectinib, LCL161, Lefamulin, Letermovir, Lumateperone, Lurasidone, M100240, Mibefradil, Netupitant, obeticholic acid, Olaparib, Osilodrostat, Palbociclib, Pazopanib, Posaconazole, Propiverine, Ravuconazole, Ribociclib, Rimegepant, Roxithromycin, Rucaparib, Schisandra sphenanthera, Scutellarin (Breviscapine), Selpercatinib, Simeprevir, Suvorexant, Tabimorelin, Tacrolimus, Telaprevir, Teriflunomide, Tofisopam, Tucatinib, Verapamil and Voxelotor.

21. Compound 1 for use, use of compound 1 or method according to any of claims 1, 2, 3, 4, 5, 6, 7, 8, 9, 10, 11, 12, 13, 14, 15, 19 or 20, or the kit according to claim 17, 18, 19 or 20, wherein the CYP inhibitor is a strong CYP3A4 inhibitor and is one or more medicaments selected from Boceprevir, Clarithromycin, Cobicistat, Conivaptan, Danoprevir, Elvitegravir, Idelalisib, Indinavir, Itraconazole, Ketoconazole, Lonafarnib, Lopinavir, Nefazodone, Nelfinavir, Posaconazole, Ribociclib, Ritonavir, Saquinavir, Telaprevir, Telithromycin, Tipranavir, Troleandomycin, Voriconazole, Ceritinib, grapefruit juice, LCL161, Mibefradil and Tucatinib.

22. Compound 1 for use, use of compound 1 or method according to any of claims 1, 2, 3, 4, 5, 6, 7, 8, 9, 10, 11, 12, 13, 14, 15, 19, 20 or 21, or the kit according to claim 17, 18, 19, 20 or 21, wherein the P-gp inhibitor is one or more medicaments selected from Amiodarone, Azithromycin, Cannabidiol, Capmatinib, Carvedilol, Clarithromycin, Cobicistat, Cyclosporine, Daclatasvir, Diosmin, Dronedarone, Elagolix, Elagolix-Estradiol-Norethindrone, Eliglustat, Elexacaftor-tezacaftor-ivacaftor, Erythromycin, Flibanserin, Fostamatinib, Glecaprevir-pibrentasvir, Ketoconazole, Itraconazole, Ivacaftor, Ketoconazole, Lapatinib, Ledipasvir, Levoketoconazole, Neratinib, Ombitasvir-paritaprevir-ritonavir, Osimertinib, Propafenone, Quinidine, Quinine, Ranolazine, Ritonavir, Rolapitant, Roxithromycin, Simeprevir, Tamoxifen, Telithromycin, Tepotinib, Tezacaftor-Ivacaftor, Ticagrelor, Tucatinib, Velpatasvir, Vemurafenib, Verapamil, and Voclosporin.

23. Compound 1 for use, use of compound 1 or method according to any of claims 1, 2, 3, 4, 5, 6, 7, 8, 9, 10, 11, 12, 13, 14, 15, 19, 20, 21 or 22, or the kit according to claim 17, 18, 19, 20, 21 or 22, wherein the P-gp inhibitor is a strong P-gp inhibitor and is one or more medicaments selected from Amiodarone, Azithromycin, Clarithromycin, Erythromycin, Roxithromycin, Telithromycin, Cyclosporine, Itraconazole, Ketoconazole, Tamoxifen, and Verapamil.

24. Compound 1 for use, use of compound 1 or method according to any of claims 1, 2, 3, 4, 5, 6, 7, 8, 9, 10, 11, 12, 13, 14, 15, 19, 20, 21, 22 or 23, or the kit according to claim 17, 18, 19, 20, 21, 22 or 23, wherein the one or more compounds that are CYP inhibitors and/or P-gp inhibitors are combined CYP3A4/P-gp inhibitors, in particular Itraconazole.

25. A pharmaceutical unit dosage composition comprising 80 mg to 200 mg of compound 1: 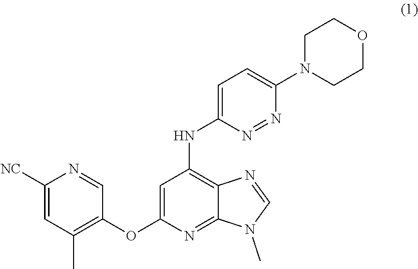 [SMILES: Cc1cc(C#N)ncc1Oc1cc(Nc2ccc(N3CCOCC3)nn2)c2ncn(C)c2n1]
or a pharmaceutically acceptable salt/cocrystal thereof, or a solvate or the solvate of a salt/cocrystal thereof, wherein the unit dosage form is suitable for oral administration up to a maximum total dosage of 200 mg of compound 1 per day.

26. The dosage form of claim 25 comprising from 90 mg to 200 mg of compound 1 in unit dosage form.

27. The dosage form of claim 25 or 26 comprising from 90 mg, 100 mg, 110 mg, 120 mg, 125 mg, 130 mg, 140 mg, 150 mg, 160 mg, 170 mg, 175 mg, 180 mg, 190 mg or 200 mg of compound 1 in unit dosage form.

28. The dosage form according to any of claims 25, 26 or 27, wherein the unit dosage is in a form selected from a liquid, a tablet, a capsule, or a gelcap.

29. The dosage form according to any of claims 25, 26, 27 or 28, wherein the unit dosage is in the form of a tablet or capsule.

30. The dosage form according to any of claims 25, 26, 27, 28 or 29 for use in the treatment of an inflammatory disease, a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, ulcerative colitis and/or Crohn's disease.

31. The use of the dosage form according to any of claims 25, 26, 27, 28 or 29, in the manufacture of a medicament for the treatment of an inflammatory disease, a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, ulcerative colitis and/or Crohn's disease.

32. A method of treating an inflammatory disease, a disease associated with hypersecretion of IFNα and/or interferons (“interferonopathies”, especially type I interferonopathies), IL-12 and/or IL-23, in particular a disease selected from systemic lupus erythematosus, cutaneous lupus erythematosus, lupus nephritis, dermatomyositis, polymyositis, Sjogren's syndrome, psoriasis, rheumatoid arthritis, psoriatic arthritis, ulcerative colitis and/or Crohn's disease, comprising administering to a patient a dosage form according to any of claims 25, 26, 27, 28 or 29.
